SEQUENCE LISTING (26 sequences)

SEQ ID NO: 1 - protein, 37 aa, Artificial Sequence
  Synthetic
DWLKAFYDKVAEKLKEAFPDWAKAAYDKAAEKAKEAA

SEQ ID NO: 2 - protein, 26 aa, Artificial Sequence
  Synthetic
WDRVKDLATVYVDVLKDSGRDYVSQF

SEQ ID NO: 3 - protein, 21 aa, Artificial Sequence
  Synthetic
LKLLDNWDSVTSTFSKLREQL

SEQ ID NO: 4 - protein, 22 aa, Artificial Sequence
  Synthetic
PVTQEFWDNLEKETEGLRQEMS

SEQ ID NO: 5 - protein, 11 aa, Artificial Sequence
  Synthetic
KDLEEVKAKVQ

SEQ ID NO: 6 - protein, 22 aa, Artificial Sequence
  Synthetic
PYLDDFQKKWQEEMELYRQKVE

SEQ ID NO: 7 - protein, 22 aa, Artificial Sequence
  Synthetic
PLRAELQEGARQKLHELQEKLS

SEQ ID NO: 8 - protein, 22 aa, Artificial Sequence
  Synthetic
PLGEEMRDRARAHVDALRTHLA

SEQ ID NO: 9 - protein, 22 aa, Artificial Sequence
  Synthetic
PYSDELRQRLAARLEALKENGG

SEQ ID NO: 10 - protein, 22 aa, Artificial Sequence
  Synthetic
ARLAEYHAKATEHLSTLSEKAK

SEQ ID NO: 11 - protein, 11 aa, Artificial Sequence
  Synthetic
PALEDLRQGLL

SEQ ID NO: 12 - protein, 22 aa, Artificial Sequence
  Synthetic
PVLESFKVSFLSALEEYTKKLN

SEQ ID NO: 13 - protein, 24 aa, Artificial Sequence
  Synthetic
TVLLLTICSLEGALVRRQAKEPCV

SEQ ID NO: 14 - protein, 12 aa, Artificial Sequence
  Synthetic
QTVTDYGKDLME

SEQ ID NO: 15 - protein, 20 aa, Artificial Sequence
  Synthetic
KVKSPELQAEAKSYFEKSKE

SEQ ID NO: 16 - protein, 24 aa, Artificial Sequence
  Synthetic
VLTLALVAVAGARAEVSADQVATV

SEQ ID NO: 17 - protein, 22 aa, Artificial Sequence
  Synthetic
NNAKEAVEHLQKSELTQQLNAL

SEQ ID NO: 18 - protein, 26 aa, Artificial Sequence
  Synthetic
LPVLVVVLSIVLEGPAPAQGTPDVSS

SEQ ID NO: 19 - protein, 21 aa, Artificial Sequence
  Synthetic
ALDKLKEFGNTLEDKARELIS

SEQ ID NO: 20 - protein, 22 aa, Artificial Sequence
  Synthetic
VVALLALLASARASEAEDASLL

SEQ ID NO: 21 - protein, 25 aa, Artificial Sequence
  Synthetic
HLRKLRKRLLRDADDLQKRLAVYQA

SEQ ID NO: 22 - protein, 22 aa, Artificial Sequence
  Synthetic
AQAWGERLRARMEEMGSRTRDR

SEQ ID NO: 23 - protein, 20 aa, Artificial Sequence
  Synthetic
LDEVKEQVAEVRAKLEEQAQ

SEQ ID NO: 24 - protein, 18 aa, Artificial Sequence
  Synthetic
DWLKAFYDKVAEKLKEAF

SEQ ID NO: 25 - protein, 23 aa, Artificial Sequence
  Synthetic
VLESFKVSXLSAXEEYTKKLNTQ

SEQ ID NO: 26 - protein, 23 aa, Artificial Sequence
  Synthetic
VLESFKVSXLSALEEXTKKLNTQ

(19) United States
(12) Patent Application Publication
(10) Pub. No.: US 2016/0074473 A1
(43) Pub. Date: Mar. 17, 2016

(54) EFFECT OF PHOSPHOLIPID COMPOSITION OF RECONSTITUTED HDL ON ITS CHOLESTEROL EFFLUX AND ANTI-INFLAMMATORY PROPERTIES

(71) Applicants: Kinemed, Inc., Emeryville, CA (US); THE UNITED STATES OF AMERICA, as represented by the Secretary, Department of Health and Human Serv, Rockville, MD (US)

(72) Inventors: Scott TURNER, Emeryville, CA (US); Anna Schwendeman, Superior Township, MI (US); Alan Remaley, Bethesda, MD (US)

(73) Assignees: Kinemed, Inc., Emeryville, CA (US); THE UNITED STATES OF AMERICA, as represented by the Secretary, Department of Health and Human Serv, Rockville, MD (US)

(21) Appl. No.: 14/815,683

(22) Filed: Jul. 31, 2015

Related U.S. Application Data
(60) Provisional application No. 62/031,705, filed on Jul. 31, 2014.

(51) Int. Cl. A61K 38/17; A61K 47/24

(52) U.S. Cl. CPC A61K 38/1709; A61K 47/24

(57) ABSTRACT

The present invention relates to peptide-phospholipid formulations, methods of generating these formulations and methods of administering these formulations for treatment. The present disclosure also provides methods for increasing cholesterol efflux, inducing anti-atherosclerotic activity, increasing pre-β HDL, inducing anti-inflammatory activity, inhibiting cytokine release (including cytokines TNF-α, IL-1β, and/or IL-6 or a combination thereof) and increasing cholesterol mobilization and/or esterification by administering the peptide-phospholipid formulations disclosed.

### CROSS REFERENCE TO RELATED APPLICATION

This application claims priority to U.S. Provisional Application No. 62/031,705, filed Jul. 31, 2014, which is incorporated herein by reference in its entirety for all purposes.

### STATEMENT AS TO FEDERALLY SPONSORED RESEARCH

This invention was made with governmental support under grant nos. R01 GM113832, R01 HL068878, R01 HL117491, T32 GM008353 all awarded by the The United States of America, as represented by the Secretary, Department of Health and Human Services (National Institutes of Health). The government has certain rights in the invention.

### FIELD OF THE INVENTION

The present invention relates to peptide-phospholipid formulations (including pharmaceutical formulations), methods of generating these peptide-phospholipid formulations and methods of administering these peptide-phospholipid formulations for treatment.

### BACKGROUND

Apolipoprotein mimetic peptides are being investigated as possible therapeutic agents for the treatment of cardiovascular disease (Remaley, Thomas et al. 2003; Osei-Hwedieh, Amar et al. 2011), as well as other disorders associated with inflammation (Osei-Hwedieh, Amar et al. 2011; Yao, Oai et al. 2011). See also U.S. Patent Application Publication Nos. 2009/0276331 and 2010/0203099, both incorporated by reference in their entireties. These peptides reduce atherosclerosis in animal models and appear to be safe in early stage clinical trials (Wool, Reardon et al. 2008; Navab, Reddy et al. 2011; Watson, Weissbach et al. 2011).

Apolipoprotein mimetic peptides have similar biological properties as full length apolipoproteins, such as apoA-I, the main protein in High Density Lipoprotein (HDL). Intravenous infusion, once a week for 4-5 weeks, of recombinant or purified apoA-I reconstituted with phospholipids has been shown to reduce plaque volume in patients with acute coronary syndrome to a degree similar to that observed after several years of statin treatment (Nissen 2005; Tardif, Gregoire et al. 2007). A major limitation of the use of apoA-I is the cost to produce the large quantities needed for this type of treatment and hence the interest in the use of short synthetic mimetic peptides, which are potentially more economical to produce (Osei-Hwedieh, Amar et al. 2011). Another potential advantage of apolipoprotein mimetic peptides is that, when they are synthesized with D-amino acids, such as the D4F peptide, they are resistant to proteolysis and can reduce atherosclerosis in animal models when given orally (Buga, Frank et al. 2006; Bloedon, Dunbar et al. 2008). Clinical development of the D4F peptide, however, has been halted because of the potential for long-term tissue accumulation (Watson, Weissbach et al. 2011).

ApoA-1 and apolipoprotein mimetic peptides potentially have several different beneficial effects in preventing or reducing atherosclerosis (Osei-Hwedieh, Amar et al. 2011), such as decreasing inflammation, oxidation and sequestering oxidized lipids. The best understood and possibly the central mechanism behind many of the beneficial properties of apoA-I is based on its ability to increase reverse cholesterol transport pathway (Yasuda, Ishida et al., 2010; Yvan-Charvet, Wang et al. 2010), which promotes the removal of excess cholesterol from peripheral cells, such as macrophages, and delivers it to the liver for excretion.

It was recently shown that the ability of HDL in serum to efflux cholesterol from macrophages was, in fact, a better predictor of the atheroprotective effect of HDL than the cholesterol content of HDL (HDL-C) (Khera, Cuchel et al. 2011), the current routine diagnostic test for assessing HDL. One of the first steps in the efflux of cholesterol from cells involves the interaction of apoA-1 with the ABCA1 transporter, followed by a detergent-like extraction step whereby apoA-1 removes cholesterol and phospholipids from cells and forms a small nascent HDL particle (Lund-Katz and Phillips 2010). A key structural motif that is necessary for this process to occur is the presence of an amphipathic alpha helix (Remaley, Thomas et al. 2003; Brewer, Remaley et al. 2004), which enables apoA-I or apolipoprotien mimetic peptides to bind to and remove cholesterol and other lipids from the lipid micro domain created by the ABCA1 transporter on the plasma membrane.

In the absence of any associated phospholipids, apolipoproteins do not as readily form amphipathic alpha helices (Frank and Marcel 2000; D'Souza, Stonik et al. 2010). This is particularly true for short synthetic amphipathic peptides, which largely form random coils when present in aqueous buffers, because water effectively competes with the intermolecular hydrogen bonds that stabilize alpha helices. Whether this less conformational constrained state for apolipoproteins or their mimetic peptides is beneficial or detrimental for their interaction with the ABCA1 transporter in the cholesterol efflux process is not known. The phospholipid packing membrane defects into which amphipathic peptides initially insert is relatively small (Cui, Lyman et al., 2011); therefore, it is possible that increasing the helicity of amphipathic peptide beyond a certain point may interfere with their efflux ability.

The helicity of synthetic peptides can be increased by chemically blocking the end of peptides (Remaley, Amar et al. 2008) and by making longer peptides and peptides with multiple helices (Remaley, Amar et al. 2008). This increases the cost of making such peptides and would be expected to reduce oral bioavailability of longer peptides. It was recently shown that the chemical modification of peptides with linkers also increases helix formation of peptides and has been used to improve the immunogenicity of synthetic peptide vaccines when the antigenic epitope is present in an alpha helical region of an intact protein (Henchey, Jochim et al. 2008; Kutchukian, Yang et al. 2009). In one instance, this peptide modification involves the covalent attachment of a hydrocarbon chain to two different regions of a peptide so that a cross-link is established, thus promoting the alignment of hydrogen bonds between the carbonyl and amino groups in the peptide backbone and facilitating helix formation. This modification has also been shown to improve the membrane permeability of peptides and makes them resistant to proteolysis (Henchey, Jochim et al. 2008; Kutchukian, Yang et al. 2009; Bhattacharya, Zhang et al. 2008). The effect of this modification on apolipoproteins or their mimetic peptides on their biological properties has been decribed in U.S. Patent Publication No. 2014/0213502 and International Patent Publication No. WO 2012/149563.

Another method for increasing the helicity of apolipoproteins or their mimetic peptides is to complex them with lipids. For example, in the lipid-free state, apoA-I is only about 20% helical, but when associated with lipids, it is over 80% helical (Smith, Pownall et al. 1978). Apolipoproteins and their mimetic peptides are typically pre-complexed with phospholipids in therapeutic formulations (Remaley, Amar et al. 2008). One advantage of reconstituting apolipoproteins and their mimetic peptides with lipids is that it increases the size of the complex, thus potentially extending the half-life of the peptide in the circulation. The reconstitution with phospholipids also potentially reduces the cytotoxicity of the peptide from non-specific lipid extraction and may enable the peptide to efflux cholesterol by other transporters besides ABCA1, such as ABCG1 and SR-BI, which primarily donate cholesterol phospholipid-rich lipoproteins (Rothblat and Phillips 2010). The reconstitution process of apolipoproteins with phospholipids, however, is relatively complex. Most methods are also not scalable, and the reconstitution process significantly adds to the cost of preparing GMP grade material that is suitable for being used as a therapy in humans.

Hydrocarbon chains similar to the acyl group of phospholipids can be covalently attached to peptides during synthesis (Nestor 2009). When apoA-I is fully lipidated with phospholipid, as in the case of when it is bound to HDL, it loses, however, its ability to interact with the ABCA1 transporter (Rothblat and Phillips 2010). The effect of hydrocarbon chain modification on apolipoproteins or their mimetic peptides on their biological properties has been described in U.S. Patent Publication No. 2014/0213502 and International Patent Publication No. WO 2012/149563.

Infusions of cholesterol-free reconstituted high-density lipoprotein (rHDL) particles have been shown to rapidly reverse atherosclerosis in a wide variety of animal models and in clinical trials of acute coronary syndrome (ACS) patients (Remaley, A. T., Amar, A., Sviridov, D. (2009) Expert Rev Cardiovasc Ther; 6(9): 1203-15; Newton, R. S., Krause, B. R. (2002) Atheroscler Suppl, 3(4): 31-8; Badimon, J. J., Badimon, L., Fuster, V. (1990) J Clin Invest, 85(4): 1234-41; Nissen, S. E., et al., (2003) JAMA, 290: 2292-2300; and Tardif, J. C., et al. (2007) JAMA; 297: 1675-1682). While a significant emphasis has been placed on investigating the HDL protein component i.e., Apolipoprotein A-I (ApoA-I), ApoA-I mutants and mimetic peptides, the importance of HDL phospholipid composition has not been systematically investigated prior to the instant disclosure. Lipid represents 50% to 80% of the total HDL mass and is known to affect particle stability in vivo, cholesterol efflux from macrophages, ability to interact with lecithin-cholesterol acyltransferase (LCAT) and cholesterol elimination (Rye, K. A., Hime, N. J., Barter, P. J. (1996) J. Biol Chem, 271: 4243-4250; Davidson, W. S., et al. (1995) J. Biol. Chem., 270: 5882-5890; Marmillot, P., Patel, S., Lakshman, M. R. (2007) Metabolism, 56(2): 251-259; Ma, C. I., et al. (2012) Biochem Cell Biol, 90(5):636-45; Sparks, D. L., et al. (1995) J. Biol. Chem., 270:26910-26917; and Parks, J. S., et al. (2000) J Lipid Res, 41: 546-553). Lipid composition also largely defines the size, net charge and rigidity of the rHDL particles—all are important factors in the pharmacokinetic and pharmacodynamics properties of rHDL. The investigation of the effects of lipid composition on the resulting rHDL properties in vitro and in vivo is the focus of the present. By better defining the lipid effect of rHDL, the present disclosure provides for formulations which alter the potency and safety of rHDL, and ultimately advance clinical translation of these potentially life-changing nanomedicines.

The phospholipid composition of endogenous HDL contains phosphatidylcholines (PC), sphingomyelin (SM) and small amounts of lysophosphatidylcholine (LPC), phosphatidylethanolamine (PE), phosphatidylinositol (PI) and other lipids (Kontush, A., Lhomme, M., Chapman, M. J. (2013) J Lipid Res, 54:2950-2962; Camont, L., et al. (2013) Arterioscler Thromb Vasc Biol, 33(12):2715-2723; and Kontush A., et al. (2007) Arterioscler Thromb Vase Biol., 27:1843-1849). The differences in lipid composition of various subclasses of HDL and how they relate to disease have been gaining attention in recent years (Kontush, A., Lhomme, M., Chapman, M. J. (2013) J Lipid Res, 54:2950-2962; Camont, L., et al. (2013) Arterioscler Thromb Vasc Biol, 33(12): 2715-2723; and Kontush A., et al. (2007) Arterioscler Thromb Vasc Biol., 27: 1843-1849). However, a single source of lipid is used for preparation of reconstituted HDL for most academic research and clinical development (Newton, R. S., Krause, B. R. (2002) Atheroscler Suppl, 3(4): 31-8; Badimon, J. J., Badimon, L., Fuster, V. (1990) J Clin Invest, 85(4): 1234-41; Nissen, S. E., et al. (2003) JAMA, 290: 2292-2300; Tardif, J. C., et al. (2007) JAMA; 297: 1675-1682; Carlson, L. (1995) Nutr Metab Cardiovasc Dis, 5:85-91; Nanjee, M. N., et al. (1999) Arterioscler Thromb Vasc Biol, 19(4): 979-89; Marchesi, M., et al. (2004) J Pharmacol Exp Ther, 311(3):1023-31; and Hippalgaonkar, K., Majumdar, S., Kansara, V. (2010) AAPS PharmSciTech, 11(4):1526-1540). The first lipid used to prepare rHDLs for human testing was soybean phosphatidylcholine (soyPC), because this lipid was used in parenteral nutrition products and was readily commercially available in infusion grade (Hippalgaonkar, K., Majumdar, S., Kansara, V. (2010) 11(4):1526-1540). Thus recombinant proapolipoprotein A-I (a product of UCB) and blood purified ApoA-I (CSL-111, a products of CSL Behring) were reconstituted with soyPC (Carlson, L. (1995) Nutr Metab Cardiovasc Dis., 5:85-91; and Nanjee, M. N., Doran, J. E., Lerch, P. G., Miller, N. E. (1999) Arterioscler Thromb Vasc Biol., 19(4):979-89). As more infusion grade chemically defined lipids became commercially available, the phospholipid composition used in the preparation of rHDL diversified. A 1-palmitoyl-2-oleoyl-sn-glycero-3-phosphocholine (POPC) was first used in ETC-216 (rApoA-I-Milano), and then a mixture of 1,2-dipalmitoyl-sn-glycero-3-phosphocholine (DPPC) and SM was used for ETC-642, a peptide-based rHDL (Marchesi, M., et al. (2004) J Pharmacol Exp Ther., 311(3): 1023-31; and Di Bartoloa, B. A., et al. (2011) Atherosclerosis, 217: 395-400). Most recently, SM was used in CER-001, another newly developed rApoA-I—based rHDL (U.S. Patent Application No. PCT/US2012/024020, 2012). Both sphingomyelin and saturated fatty acid containing lipids like DPPC have superior cholesterol binding capacity relative to unsaturated POPC and soyPC (Ohvo-Rekilä, H., et al. (2002) Prog Lipid Res, 41(1): 66-97; and Ramstedt, B., Slotte, J. P. (1999) Biophys. J. 76: 908-915). In fact, SM has the strongest cholesterol binding affinity governed by Van der Waals' interaction due to the mismatched fatty acid lengths (Ohvo-Rekilä, H., et al. (2002) Prog Lipid Res, 41(1): 66-97). SM is also known to form lipid-raft sections in cellular membrane that contain up to 50% of cholesterol (Silva, L. C., Futerman, A. H., Prieto, M. (2009) Biophys. J. 96(8): 3210-22). The rHDLs made from saturated lipids exhibited greater cholesterol efflux from macrophages in vitro (Marmillot, P., Patel, S., Lakshman, M. R. (2007) Metabolism, 56(2): 251-9; and Ma, C. I., et al. (2012) Biochem Cell Biol, 90(5): 636-45) and cholesterol mobilization in vivo (International Patent Publication No. WO 2003/096983). However, unsaturation enhances rHDL interaction with LCAT (Parks, J. S., et al. (2000) J Lipid Res., 41: 546-553), whereas SM was reported to inhibit cholesterol esterification in vitro (Rye, K. A., Hime, N. J., Barter, P. J. (1996) J. Biol Chem., 271: 4243-4250).

The present disclosure describes whether higher cholesterol binding affinity of SM relative to POPC would translate into greater cholesterol efflux both in vitro and in vivo. The ApoA-I mimetic, 5A, a 37-amino acid-long bi-helical peptide (Sethi, A. A., et al. (2008) J Biol Chem., 283:32273-32282), was used to form SM and POPC-based HDL particles. The 5A based HDL has several similar features to HDL prepared from the full-length ApoA-I, namely, an equivalent ability to promote cholesterol efflux in vitro and in vivo (Sethi, A. A., et al. (2008) J Biol Chem., 283:32273-32282; and Amar, M. J. A., et al. (2010) J. Pharmacol. Exp. Ther., 334: 634-641), and similar abilities in reducing the development of atherosclerosis (Amar, M. J., W. D'Souza, et al. (2010) J Pharmacol Exp Ther 334(2):634-41) and suppressing inflammation (Tabet, F., et al. (2010) Arterioscler Thromb. Vasc. Biol., 30: 246-252). The differences in cholesterol efflux and inflammatory cytokine release inhibition for 5A-POPC and 5A-SM are also described. In addition, the ability to mobilize and esterify cholesterol in vivo depending on the dose and lipid composition of infused rHDL was evaluated in rats.

There remains, therefore, a need in the art for particular formulations comprising short apolipoprotein mimetic peptides complexed with phospholipids that are effective in promoting cholesterol efflux and active in the other biological properties of these peptides. The present disclosure meets this need.

### BRIEF SUMMARY OF THE INVENTION

The present disclosure provides pharmaceutical formulations comprising the apolipoprotein mimetic peptide 5A (SEQ ID NO:1) and at least one phospholipid (i.e., pharmaceutical formulations comprising peptide-phospholipids). In some embodiments, the at least one phospholipid is sphingomyelin (SM) and/or 1-palmitoyl-2-oleoyl-sn-glycero-3-phosphocholine (POPC) and/or a combination thereof. In some embodiments, the at least one phospholipid is sphingomyelin (SM). In some embodiments, the at least one phospholipid is 1-palmitoyl-2-oleoyl-sn-glycero-3-phosphocholine (POPC).

In some embodiments, the molar ratio of peptide to phospholipid of 1:3, 1:4, 1:5, 1:6, 1:7, 1:8, 1:9, or 1:10. In some embodiments, the molar ratio of peptide to phospholipid is 1:7.

In some embodiments, the peptide is complexed with said at least one phospholipid.

In some embodiments, the formulation is lyophilized.

In some embodiments, the formulation has a pH of 5 to 7. In some embodiments, the formulation has a pH of 5, 5.5, 6, 6.5, or 7.

The present disclosure also provides methods for generating a peptide-phospholipid pharmaceutical formulation including the peptide-phospholipids disclosed. In some embodiments, the method comprises mixing of said peptide with a phospholipid, wherein said mixing occurs at a pH of 5 to 7. In some embodiments, the pH at mixing is 5, 5.5, 6, 6.5, or 7. In some embodiments, the peptide and the at least one phospholipid are mixed at a 1:3, 1:4, 1:5, 1:6, 1:7, 1:8, 1:9, or 1:10 molar ratio of peptide to phospholipid. In some embodiments, the molar ratio of peptide to phospholipid during mixing is 1:7.

In some embodiments, the method further includes a step of lyophilization.

In some embodiments, the method generates a high-density lipoprotein-like complex.

The present disclosure also provides methods for treating a subject in need thereof comprising administering a peptide-phospholipid pharmaceutical formulation as disclosed herein.

The present disclosure also provides methods for increasing cholesterol efflux in a subject in need thereof comprising administering a peptide-phospholipid pharmaceutical formulation as disclosed herein.

The present disclosure also provides methods for treating a subject in need thereof comprising administering a pharmaceutical formulation as described herein, wherein said peptide-phospholipid pharmaceutical formulation exhibits anti-atherosclerotic activity.

The present disclosure also provides methods for increasing pre-β HDL in a subject in need thereof comprising administering a peptide-phospholipid pharmaceutical formulation as described herein.

The present disclosure also provides methods for treating a subject in need thereof comprising administering a pharmaceutical formulation as described herein, wherein said peptide-phospholipid pharmaceutical formulation exhibits anti-inflammatory activity.

The present disclosure also provides methods for treating a subject in need thereof comprising administering a pharmaceutical formulation as described herein, wherein said peptide-phospholipid pharmaceutical formulation inhibits cytokine release. In some embodiments, the cytokines inhibited include TNF-α, IL-1β, and/or IL-6 and/or a combination thereof.

The present disclosure also provides methods for increasing cholesterol mobilization and/or esterification in a subject in need thereof comprising administering a peptide-phospholipid pharmaceutical formulation as described herein.

The present disclosure also provides methods for inducing atherosclerosis regression in a subject in need thereof comprising administering a peptide-phospholipid pharmaceutical formulation as described herein.

The present disclosure also provides methods for treatment as described herein, which further include administering an additional agent before, after, or concurrently with the peptide-phospholipid pharmaceutical formulation as described herein.

### BRIEF DESCRIPTION OF THE DRAWINGS

FIG. 1. Gel permeation chromatographs of 5A-SM and 5A-POPC. Transmission electron microscopy (TEM) images of 5A-SM (A), 5A-POPC (B), purified human HDL (C) and a mixture of 5A-SM and purified human HDL (D). Images were obtained using JEM 1200EX electron microscope at 150,000-fold magnification.

FIG. 2. Transmission electron microscopy (TEM) of rHDL. Gel permeation chromatograms of 5A-SM (A) and 5A-POPC (B). HDL peak (10 min retention time, RT) is separated from free peptide (19 min RT) and liposome (7.5 min RT) using Tosoh TSK gel G3000SWx1 and detected by UV 220 nm absorption.

FIG. 3. Cholesterol efflux dependence on peptide concentration. Concentration dependence of cholesterol efflux by 5A-SM (circles), 5A-POPC (triangles) and 5A peptide (squares). Percent of cholesterol efflux was determined after 18 h incubation of plasma with BHK cells stably transfected with ABCA1 (A), ABCG1 (B), SR-BI (C) transporters and mock cells (D). Statistically significant differences between 5A-SM and 5A-POPC (*) of p values at least <0.5.

FIG. 4. Cholesterol efflux dependence on time. Cholesterol efflux capacity of murine plasma at different times following administration of 60 mg/kg of 5A-SM (circles) and 5A-POPC (triangles). Percent of cholesterol efflux was determined after 18-h incubation of plasma with BHK cells stably transfected with human ABCA1 (A), ABCG1 (B), and SR-BI (C) transporters and mock cells (D). Statistically significant differences between 5A-SM and 5A-POPC (*) of p values at least <0.5.

FIG. 5. 5A-POPC and 5A-SM causes HDL remodeling. Human plasma incubation of 5A-POPC and 5A-SM causes HDL remodeling. (A) Dose dependent formation of smaller pre-β like HDL (arrow) upon 5A-POPC and 5A-SM incubation with plasma at 0.05, 1 and 0.5 mg/mL relative to PBS control. (B) Quantitative analysis of pre-β HDL formation in each lane. Statistically significant increase in pre-β HDL formation of 5A-POPC and 5A-SM relative to control (* p<0.5). More pre-β is formed following incubation with 5A-SM relative to 5A-POPC (** p<0.5).

FIG. 6. 2-D gel electrophoresis of human plasma. 2-D gel electrophoresis of human plasma incubated with PBS control (A) and 0.5 mg/mL of 5A-POPC (B) and 5A-SM (C). Map of various sub-classes of HDL according to classification by Asztalos et al ((2011) Curr Opin Lipidol, 22(3): 176-185) (D).

FIG. 7. Inhibition of cytokine release by 5A:POPC and 5A:SM incubation. Inhibition of cytokine release by 5A-POPC and 5A-SM incubation. (A) TNF-α release from murine macrophages stimulated by addition of 1 μg/mL LPS was inhibited by pre-incubation with 5A-SM at 1 and 0.1 mg/mL and partially inhibited by 5A-POPC at 1 mg/mL. (B) Inhibition of cytokines release in whole human blood following PHA stimulation by addition of 0.1 mg/mL of 5A-POPC (white bars) and 5A-SM (solid bars) relative to saline control (lined bars). Statistically significant differences between rHDL and PBS control (*) and between 5A-SM and 5A-POPC (**) of p values at least <0.5.

FIG. 8. Dose response of cholesterol mobilization following 5A-POPC and 5A-SM infusions. Dose response of cholesterol mobilization following 5A-POPC and 5A-SM infusions at 10 (squares), 30 (triangles) and 100 (circles) mg/kg in normal rats. Unesterified or free cholesterol mobilization by 5A-POPC (A) and 5A-SM (B). Increase in cholesterol ester (CE) levels after infusion of 5A-POPC (C) and 5A-SM (D). Statistical significant differences between 5A-POPC and 5A-SM (*) with p-values of at least <0.5.

FIG. 9. Electron microscopy and particle size distribution. Electron microscopy images of 5A-SM (A), 5A-POPC (B) and particle size distribution analyzed by dynamic light scattering for 5A-SM (C), 5A-POPC (D). The size bar corresponds to 0.1 μm.

FIG. 10. Cholesterol efflux dependence on peptide concentration. Concentration dependence of cholesterol efflux by 5A:SM (solid line), 5A:POPC (dashed line) and 5A peptide (dotted line). Percent of cholesterol efflux was determined after 18 hr incubation of plasma with BHK cells stably transfected with (A) ABCA1, (B) ABCG1, (C) SR-BI transporters or (D) mock cells. Statistically significant differences between 5A-SM and 5A-POPC (*) of p values at least <0.05.

FIG. 11. 5A-POPC and 5A-SM causes HDL remodeling. Human plasma incubation of 5A-POPC and 5A-SM causes HDL remodeling. (A) Dose dependent formation of smaller pre-β like HDL (arrow) upon 5A-POPC and 5A-SM incubation with plasma at 0.05, 1 and 0.5 mg/mL relative to PBS control. (B) Quantitative analysis of pre-β HDL formation in each lane. Statistically significant increase in pre-β HDL was observed for incubation with 0.5 mg/mL of both rHDL relative to control (*, p<0.05). More pre-β is formed following incubation with 5A-SM relative to 5A-POPC (**, p<0.05)

FIG. 12. 2-D gel electrophoresis of rat and human plasma after incubation with rHDL. 2-D gel electrophoresis of rat (A) and human plasma (B) incubated with either PBS control, 0.5 mg/mL of 5A-POPC, or 5A-SM. Pre-β HDL is highlighted by dashed line circle. (C) Map of various subclasses of HDL according to classification by Asztalos et al (Asztalos, B. F., Tani, M, Schaefer, E. J. (2011) Curr Opin in Lipidology, 22:176-185).

FIG. 13. Inhibition of cytokine release by 5A-POPC and 5A-SM incubation Inhibition of cytokine release by 5A-POPC and 5A-SM incubation. (A) TNF-α release from murine macrophages stimulated by addition of 1 LPS was inhibited by pre-incubation with 5A-SM at 1 and 0.1 mg/mL and partially inhibited by 5A-POPC at 1 mg/mL. (B) Inhibition of cytokines release in whole human blood following PHA stimulation by addition of 0.1 mg/mL of 5A-POPC (white bars) and 5A-SM (solid bars) relative to saline control (lined bars). Statistically significant differences between rHDL and PBS control (*) and between 5A-SM and 5A-POPC (**) of p values at least <0.05.

FIG. 14. Dose response of cholesterol mobilization following 5A-POPC and 5A-SM infusions. Dose response of cholesterol mobilization following 5A-POPC and 5A-SM infusions at 30 mg/kg (dashed line) and 100 mg/kg (solid line) in normal rats. Unesterified or free cholesterol mobilization by 5A-POPC (A) and 5A-SM (B). Increase in cholesterol ester (CE) levels after infusion of 5A-POPC (C) and 5A-SM (D). Increase in phospholipids after infusion of 5A-POPC (E) or 5A-SM (F). (*) denotes statistical significant differences between 5A-POPC and 5A-SM of same dose with p-values of at least <0.05.

FIG. 15. Infusion of 5A-POPC and 5A-SM leads to rapid cholesterol mobilization in the HDL sub-fraction. Infusion of 5A-POPC (dotted line) and 5A-SM (dashed line) leads to rapid cholesterol mobilization in the HDL sub-fraction 30 min post-dose relative to baseline (solid line). Lipoproteins were separated by gel filtration chromatography and cholesterol levels were analyzed post fraction collection. Peaks at 12, 14, and 18 minutes represent VLDL, LDL, and HDL, respectively.

FIG. 16. Effect of 5A-POPC and 5A-SM rHDL on atherosclerosis regression in ApoE−/− mice. Effect of 5A-POPC and 5A-SM rHDL on atherosclerosis regression in ApoE−/− mice. Aortas were dissected and plaque areas were visualized by oil red O staining Representative lesions images and corresponding quantitative analyses of the aortas (A, B) and the aortic root cross-sections (C, D). N=7-8 animals per group, (*) denotes statistically significant differences with p-values of at least <0.05; (**) indicates p-values <0.01.

FIG. 17. Gel permeation chromatography analysis of rHDL particles 5ASM (A) and 5A-POPC (B). The 5A-SM and 5A-POPC complexes were analyzed by gel permeation chromatography, with UV detection at 220 nm, using Tosoh TSK gel G3000SWx1 column (Tosoh Bioscience, King of Prussia, Pa.). The peaks around 7 minutes and 12 minutes correspond to rHDL and free 5A peptide, respectively.

### DETAILED DESCRIPTION OF THE INVENTION

Apolipoproteins are proteins that bind lipids and transport the lipids through the lymphatic and circulatory systems. There are six classes of apolipoproteins and several sub-classes: A (Apo AI, Apo A-II, Apo A-IV and Apo A-V), B (Apo B48 and Apo B100), C (Apo C-I, Apo C-II, Apo C-III and Apo C-IV), D, E and H. In some embodiments, the apolipoprotein of the invention is 5A (DWLKAFYDKVAEKLKEAFPDWAKAAYDKAAEKAKEAA; SEQ ID NO:1).

Apolipoproteins can be divided into two categories based on three dimensional and functional differences. Apolipoprotein B, for example, forms low-density lipoprotein (“bad cholesterol”) particles. These proteins have mostly beta-sheet structure and associate with lipid droplets irreversibly.

Other apolipoproteins form high-density lipoprotein (HDL) (“good cholesterol”) particles. These proteins contain alpha-helices and associate with lipid droplets reversibly. HDL apolipoproteins remove cellular cholesterol and phospholipids by a cholesterol-inducible active transport process mediated by a cell membrane protein called ATP-binding cassette transporter A1 (ABCA1).

The last helix of apoA-I has been shown to be critical in the ability of the full-length protein to promote cholesterol efflux, but when synthesized as a single helical peptide, it is unable to promote cholesterol efflux (Panagotopulos, Witting et al. 2002). As described in U.S. Patent Publication No. 2014/0213502 and International Patent Publication No. WO 2012/149563, hydrocarbon chain stapling of the last helix of apoA-I results in improved cholesterol efflux from cells relative to unstapled their unstapled counterparts.

A “mimetic peptide,” mimic or “peptidomimetic” means a mimetic of a peptide which includes some alteration of the normal peptide chemistry. Peptidomimetics typically enhance some property of the original peptide, such as increased stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. The peptides and peptides in the peptide-phospholipid forlmulations described herein include mimetic peptides.

Use of peptidomimetics can involve the incorporation of a non-amino acid residue with non-amide linkages at a given position. As disclosed in U.S. Patent Publication No. 2014/0213502 and International Patent Publication No. WO 2012/149563, peptidomimetics include the compounds with a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

In some embodiments, two or more hydrocarbon chain staples can be used to stabilize the helicity of peptides, such hydrocarbon chain staples are disclosed in U.S. Patent Publication No. 2014/0213502 and International Patent Publication No. WO 2012/149563.

“Stapled,” “Stapling” and “hydrocarbon-stapling” refer to the introduction into a peptide of at least two moieties capable of undergoing reaction to promote carbon-carbon bond formation when contacted with a reagent to generate at least one cross-linker between the at least two moieties.

“Peptide” or “Polypeptide” refers to any oligopeptide, polypeptide, gene product, expression product, or protein. A polypeptide is comprised of consecutive amino acids. The term “polypeptide” encompasses naturally occurring or synthetic molecules. The term “polypeptide” refers to amino acids joined to each other by peptide bonds or modified peptide bonds, e.g., peptide isosteres, etc. and may contain modified amino acids other than the 20 gene-encoded amino acids.

“Complexed” or “complexing” and variations thereof as used herein refer to a composition which includes at least two components wherein the at least two components interact with one another. Such interactions can include but are not limited to molecular level interactions, non-covalent interactions, and covalent interactions. Such interactions can result in complex formation, such as formation of the peptide-phospholipids disclosed herein (also referred to herein as rHDL, reconstituted high-density lipoprotein and/or reconstituted HDL).

Exemplary peptides for use in the peptide-phopholipid formulations and the peptide-phopholipid pharmaceutical formulations of the invention include:

5A peptide:
SEQ ID NO: 1
DWLKAFYDKVAEKLKEAFPDWAKAAYDKAAEKAKEAA;
ApoA-I; Helix 1; 8-33
(SEQ ID NO: 2)
WDRVKDLATVYVDVLKDSGRDYVSQF
ApoA-I; Helix 2; 44-65
(SEQ. ID. NO: 3)
LKLLDNWDSVTSTFSKLREQL
ApoA-I; Helix 3; 66-87
(SEQ. ID. NO: 4)
PVTQEFWDNLEKETEGLRQEMS
ApoA-I; Helix 4: 88-98
(SEQ. ID. NO: 5)
KDLEEVKAKVQ
ApoA-I; Helix 5; 99-120
(SEQ. ID. NO: 6)
PYLDDFQKKWQEEMELYRQKVE
ApoA-I; Helix 5; 121-142
(SEQ. ID. NO: 7)
PLRAELQEGARQKLHELQEKLS
ApoA-I; Helix 7; 143-164
(SEQ. ID. NO: 8)
PLGEEMRDRARAHVDALRTHLA
ApoA-I; Helix 8; 165-183
(SEQ. ID. NO: 9)
PYSDELRQRLAARLEALKENGG
ApoA-I; Helix 9; 187-208
(SEQ. ID. NO: 10)
ARLAEYHAKATEHLSTLSEKAK
ApoA-I; Helix 10; 209-219
(SEQ. ID. NO: 11)
PALEDLRQGLL
ApoA-I; Helix 11; 220-243
(SEQ. ID. NO: 12)
PVLESFKVSFLSALEEYTKKLN
ApoA-II; Helix 1; 7-30
(SEQ. ID. NO: 13)
TVLLLTICSLEGALVRRQAKEPCV
ApoA-II; Helix 2; 39-50
(SEQ. ID. NO: 14)
QTVTDYGKDLME
ApoA-II; Helix 3; 51-71
(SEQ. ID. NO: 15)
KVKSPELQAEAKSYFEKSKE
ApoA-IV; Helix 1; 7-31
(SEQ. ID. NO: 16)
VLTLALVAVAGARAEVSADQVATV
ApoA-IV; Helix 2; 40-61
(SEQ. ID. NO: 17)
NNAKEAVEHLQKSELTQQLNAL
ApoC-I; Helix 1: 7-32
(SEQ. ID. NO: 18)
LPVLVVVLSIVLEGPAPAQGTPDVSS
ApoC-I; Helix 2; 33-53
(SEQ. ID. NO: 19)
ALDKLKEFGNTLEDKARELIS
ApoC-III; Helix 2; 28-49
(SEQ. ID. NO: 20)
VVALLALLASARASEAEDASLL
ApoE; Helix 2; 158-182
(SEQ. ID. NO: 21)
HLRKLRKRLLRDADDLQKRLAVYQA
ApoE; Helix 4; 26-48
(SEQ. ID. NO: 22)
AQAWGERLRARMEEMGSRTRDR
ApoE; Helix 5; 249-266
(SEQ. ID. NO: 23)
LDEVKEQVAEVRAKLEEQAQ
18A synthetic consensus peptide; Helix 1; 1-18
(SEQ. ID. NO: 24)
DWLKAFYDKVAEKLKEAF
S1A10; the peptide contains a hydrocarbon bridge
between the third and fourth helical turns of two
helices by inter-linking of the two X₁ residues;
X₁ is (S)-a-(4′-pentenyl)Ala
(SEQ ID NO: 25)
VLESFKVSX₁LSAX₁EEYTKKLNTQ
S2A10; the peptide contains a hydrocarbon bridge
between the third and fifth helical turns of two
helices by inter-linking of the X₁ and X₂
residues; X₁ is (S)-a-(4′-pentenyl)Ala and X₂ is
(R)-a-(7′-octanyl)Ala
(SEQ ID NO: 26)
VLESFKVSX₂LSALEEX₁TKKLNTQ.

In some embodiments, the peptide for use in the disclosed methods and formulations includes any one of the peptides listed in SEQ ID NOs:1-26. In some embodiments, the peptides of the invention include SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21. SEQ ID NO:22, SEQ ID NO:23, SEQ ID NO:24, SEQ ID NO:25, or SEQ ID NO: 26. In some embodiments, the peptide is a mixture of 1, 2, 3, 4, 5, or more peptides.

In some embodiments, the peptide is 5A (DWLKAFYDKVAEKLKEAFPDWAKAAYDKAAEKAKEAA; SEQ ID NO:1).

In some embodiments, the peptide is amino acids 8-33 of ApoA-I (SEQ ID NO:2). In some embodiments, the peptide is amino acids 44-65 of ApoA-I (SEQ ID NO:3). In some embodiments, the peptide is amino acids 66-87 of ApoA-I (SEQ ID NO:4). In some embodiments, the peptide is amino acids 88-98 of ApoA-I (SEQ ID NO:5). In some embodiments, the peptide is amino acids 99-120 of ApoA-I (SEQ ID NO:6). In some embodiments, the peptide is amino acids 121-142 of ApoA-I (SEQ ID NO:7). In some embodiments, the peptide is amino acids 143-164 of ApoA-I (SEQ ID NO:8). In some embodiments, the peptide is amino acids 165-183 of ApoA-I (SEQ ID NO:9). In some embodiments, the peptide is amino acids 187-208 of ApoA-I (SEQ ID NO:10). In some embodiments, the peptide is amino acids 209-219 of ApoA-I (SEQ ID NO:11). In some embodiments, the peptide is amino acids 220-243 of ApoA-I (SEQ ID NO:12). In some embodiments, the peptide is amino acids 7-30 of ApoA-II (SEQ ID NO:13). In some embodiments, the peptide is amino acids 39-50 of ApoA-II (SEQ ID NO:14). In some embodiments, the peptide is amino acids 51-71 of ApoA-II (SEQ ID NO:15). In some embodiments, the peptide is amino acids 7-31 of ApoA-IV (SEQ ID NO:16). In some embodiments, the peptide is amino acids 40-61 of ApoA-IV (SEQ ID NO:17). In some embodiments, the peptide is amino acids 7-32 of ApoC-I (SEQ ID NO:18). In some embodiments, the peptide is amino acids 33-53 of ApoC-I (SEQ ID NO:19). In some embodiments, the peptide is amino acids 28-49 of ApoC-III (SEQ ID NO:20). In some embodiments, the peptide is amino acids 158-182 of ApoE (SEQ ID NO:21). In some embodiments, the peptide is amino acids 26-48 of ApoE (SEQ ID NO:22). In some embodiments, the peptide is amino acids 249-266 of ApoE (SEQ ID NO:23). In some embodiments, the peptide is a synthetic consensus peptide (SEQ ID NO:24), the peptide is a stapled peptide S1A10 (SEQ ID NO:25), or the peptide is a stapled peptide S2A10 (SEQ ID NO:26).

The term “amino acid” refers to a molecule containing both an amino group and a carboxyl group. Amino acids include alpha-amino acids and beta-amino acids. Suitable amino acids include, without limitation, natural alpha-amino acids such as D- and L-isomers of the 20 common naturally occurring alpha-amino acids (A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q glutamine; R, arginine; S, serine; T, threonine; V, valine; W, tryptophan; Y, tyrosine; Z, glutamine or glutamic acid), unnatural alpha-amino acids, natural beta-amino acids (e.g., beta-alanine), and unnatural beta-amino acids.

In some embodiments, the present invention provides peptides and peptidomimetics in which one or more amino acids is a D amino acid or a non-naturally occurring amino acid or amino acid mimetic.

One or more of the amino acids in a peptide or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A peptide or polypeptide may also be a single molecule or may be a multi-molecular complex, such as a protein. A peptide or polypeptide may be just a fragment of a naturally occurring protein or peptide. A peptide or polypeptide may be naturally occurring, recombinant, or synthetic, or any combination thereof. As used herein “dipeptide” refers to two covalently linked amino acids.

Amino acids used in the construction of peptides of the present invention may be prepared by organic synthesis, or obtained by other routes, such as, for example, degradation of or isolation from a natural source.

There are many known unnatural amino acids any of which may be included in the peptides of the present invention. See, for example, S. Hunt, The Non-Protein Amino Acids: In Chemistry and Biochemistry of the Amino Acids, edited by G. C. Barrett, Chapman and Hall, 1985. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; CH₃), disubstituted phenylalanines, substituted tyrosines (e.g., further substituted with —C(═O)C₆H₅; —CF₃; —CN; -halo; —NO₂; CH₃), and statine. Additionally, the amino acids suitable for use in the present invention may be derivatized to include amino acid residues that are hydroxylated, phosphorylated, sulfonated, acylated, and glycosylated, to name a few.

The term “amino acid side chain” refers to a group attached to the alpha- or beta-carbon of an amino acid. A “suitable amino acid side chain” includes, for example, methyl (as the alpha-amino acid side chain for alanine is methyl), 4-hydroxyphenylmethyl (as the alpha-amino acid side chain for tyrosine is 4-hydroxyphenylmethyl) and thiomethyl (as the alpha-amino acid side chain for cysteine is thiomethyl), etc.

A “terminally unsaturated amino acid side chain” refers to an amino acid side chain bearing a terminal unsaturated moiety, such as a substituted or unsubstituted, double-bond (e.g., olefinic) or a triple-bond (e.g., acetylenic), that participates in crosslinking reaction with other terminal unsaturated moieties in the polypeptide chain. In certain embodiments, a “terminally unsaturated amino acid side chain” is a terminal olefinic amino acid side chain. In certain embodiments, a “terminally unsaturated amino acid side chain” is a terminal acetylenic amino acid side chain. In certain embodiments, the terminal moiety of a “terminally unsaturated amino acid side chain” is not further substituted.

The phrase “substantially alpha-helical” refers to a polypeptide adopting, on average, backbone (ρ, φ) dihedral angles in a range from about (−90°, −15°) to about (−35°, −70°). Alternatively, the phrase “substantially alpha-helical” refers to a polypeptide adopting dihedral angles such that the φ dihedral angle of one residue and the p dihedral angle of the next residue sums, on average, about −80° to about −125°. In certain embodiments, the polypeptide adopts dihedral angles such that the φ dihedral angle of one residue and the ρ dihedral angle of the next residue sums, on average, about −100° to about −110°. In certain embodiments, the polypeptide adopts dihedral angles such that the φ dihedral angle of one residue and the ρ dihedral angle of the next residue sums, on average, about −105°. Furthermore, the phrase “substantially alpha-helical” may also refer to a polypeptide having at least 50%, 60%, 70%, 80%, 90%, or 95% of the amino acids provided in the polypeptide chain in an alpha-helical conformation, or with dihedral angles as specified herein. Confirmation of a polypeptide's alpha-helical secondary structure may be ascertained by known analytical techniques, such as x-ray crystallography, electron crystallography, fiber diffraction, fluorescence anisotropy, circular dichroism (CD), and nuclear magnetic resonance spectroscopy.

One method of producing the disclosed polypeptides is to link two or more amino acid residues, peptides or polypeptides together by protein chemistry techniques. For example, peptides or polypeptides are chemically synthesized using currently available laboratory equipment using either Fmoc (9-fluorenylmethyloxycarbonyl) or Boc (tert-butyloxycarbonoyl) chemistry (Applied Biosystems, Inc., Foster City, Calif.). A peptide or polypeptide can be synthesized and not cleaved from its synthesis resin, whereas the other fragment of a peptide or protein can be synthesized and subsequently cleaved from the resin, thereby exposing a terminal group, which is functionally blocked on the other fragment. By peptide condensation reactions, these two fragments can be covalently joined via a peptide bond at their carboxyl and amino termini, respectively, (Grant G A (1992) Synthetic Peptides: A User Guide. W.H. Freeman and Co., New York (1992); Bodansky M and Trost B., Ed. (1993) Principles of Peptide Synthesis. Springer-Verlag Inc., New York).

Non-natural amino acids containing olefin-bearing tethers may be synthesized, for example, according to methodology provided in Schafmeister et al. (J. Am. Chem. Soc., 113:9276-9286 (1991)).

Alternatively, the peptide or polypeptide is independently synthesized in vivo, such as through cellular expression methods. Cells for expression can include mammalian, yeast and bacterial cells. Once isolated, these independent peptides or polypeptides may be linked to form a peptide or fragment thereof via similar peptide condensation reactions.

Thus, in some embodiments, stapled apolipoprotein mimetic peptides that can mimic the function of an apolipoprotein are disclosed. Stapling provides a constraint on a secondary structure, such as an alpha-helical structure. Such methods and peptides have been described in U.S. Patent Publication No. 2014/0213502 and International Patent Publication No. WO 2012/149563, both of which are incorporated herein by reference in their entireties for all purposes. In accordance with those disclosures, hydrocarbon stapling of amphipathic peptides increases their helical content, reduces their susceptibility to proteolysis and increases their ability to promote cholesterol efflux by the ABCA1 transporter.

In some embodiments, one or more pairs of α, α-disubstituted non-natural amino acids containing olefin-bearing tethers corresponding to the native amino acids are substituted into the alpha-helices of the Apo derived peptide. In other embodiments, one or more pairs of α, α-disubstituted non-natural amino acids containing olefin-bearing tethers corresponded to the native amino acids are substituted into residues.

In other embodiments, other types of linkers (Henchey, Jochim et al. 2008) that stabilize the helicity of peptides are used. Suitable linkers include, without limitation, disulfides, lactams, metal mediated bridges, hydrazones, photoclick staples, cysteine staples and hydrogen bond surrogates or alternative amino acids (Henchey, Jochim et al. 2008), such as beta amino acids that promote helix formation.

In some embodiments, polar or charged linkers placed on the hydrophilic face of amphipathic apolipoprotein peptides and peptidomimetics that stabilize the helicity of peptides are employed in the peptides of the disclosure.

In other embodiments, other proteins besides apoA-J or synthetic peptides that contain amphipathic helices can be modified by chemical linkers for promoting cholesterol efflux, as well as the other biological properties of these peptides. For example, the ligand binding domains of apoE and apoB are in helical regions and promote the uptake of lipoproteins by various receptors, such as the LDL-receptor. Stabilization of the ligand binding domain of these peptides by hydrocarbon chain linkers and other types of linkers, increases the uptake of lipoproteins by cellular receptors when peptides with these modifications are associated with lipoproteins. Helical regions on apolipoproteins also act as docking sites or regulators of many different lipoprotein modifying proteins, such as Cholesteryl Ester Transfer protein, phospholipid transfer protein, lecithin:choleserol acyltransferase, lipoprotein lipase, endothelial lipase, hepatic lipase plus others. Stabilization of helical regions of apolipoproteins or their short synthetic peptide mimics by chemical linkers can also be used for promoting the interaction with these other proteins.

In other embodiments, apolipoprotein mimetic peptides and peptidomimetics are provided by the disclosure in a modified form, resulting from covalent attachment of different hydrocarbon chains of either shorter (e.g., Capric acid or Lauric acid) or longer length (e.g., Palmitic acid and Stearic Acid) and either fully saturated (e.g., Palmitc acid and Stearic acid) or unsaturated (e.g., Linolenic acid or Arachidonic acid) in either the trans or cis configuration.

In other embodiments, apolipoprotein mimetic peptides and peptidomimetics are provided by the disclosure in a modified form, resulting from covalent attachment of a hydrocarbon chain with one of a variety of chemical bonds, such as ester bonds, ether bonds, amide bonds or by direct incorporation with FMOC-amino acid derivatives containing a hydrocarbon chain, such as the ones described herein.

In other embodiments, apolipoprotein mimetic peptides and peptidomimetics are provided by the disclosure in a modified form, resulting from covalent attachment of one or more hydrocarbon chains at the amino terminal end, the carboxy terminal end or any intervening site on peptides.

As used herein, “substantially pure” means that the depicted or named compound is at least about 60% by weight. For example, “substantially pure” can mean about 60%, 70%, 72%, 75%, 77%, 80%, 82%, 85%, 86%, 87%, 88%, 89%, 90%, 91%, 92%, 93%, 94%, 95%, 96%, 97%, 98%, 99%, 99.5%, 99.9%, or a percentage between 70% and 100%. In one embodiment, substantially pure means that the depicted or named compound is at least about 75%. In a some embodiments, substantially pure means that the depicted or named compound is at least about 90% by weight.

In some embodiments, the peptides of the invention are formulated with phospholipids. In some embodiments, the peptide-phospholipids disclosed are referred to rHDL. In some embodiments, the formulation is a pharmaceutical formulation (also referred to herein as a peptide-phospholipid pharmaceutical formulation). In some embodiments, the at least one phospholipid is sphingomyelin (SM) or palmitoyl-2-oleoyl-sn-glycero-3-phosphocholine (POPC). In some embodiments, the phospholipid is a mixture of sphingomyelin (SM) and palmitoyl-2-oleoyl-sn-glycero-3-phosphocholine (POPC). In some embodiments, the at least one phospholipid is sphingomyelin (SM). In some embodiments, the phospholipid is palmitoyl-2-oleoyl-sn-glycero-3-phosphocholine (POPC). In some embodiments, the at least one phospholipid is sphingomyelin (SM) and the peptide is 5A (e.g., 5A-SM). In some embodiments, the phospholipid is palmitoyl-2-oleoyl-sn-glycero-3-phosphocholine (POPC) and the peptide is 5A (e.g., 5A-POPC).

The present disclosure also provides for particular ratios of peptide to phospholipid in the peptide-phospholipid formulation. In some embodiments, the phospholipid is included in the formulation at a 1:3 to 1:10 molar ratio of peptide to phospholipid. In some embodiments, the formulation comprises a phospholipid at a 1:3, 1:4, 1:5, 1:6, 1:7, 1:8, 1:9, or 1:10 molar ratio of peptide to phospholipid. In some embodiments, the formulation comprises a phospholipid at a 1:3 molar ratio of peptide to phospholipid. In some embodiments, the formulation comprises a phospholipid at a 1:4 molar ratio of peptide to phospholipid. In some embodiments, the formulation comprises a phospholipid at a 1:5 molar ratio of peptide to phospholipid. In some embodiments, the formulation comprises a phospholipid at a 1:6 molar ratio of peptide to phospholipid. In some embodiments, the formulation comprises a phospholipid at a 1:7 molar ratio of peptide to phospholipid. In some embodiments, the formulation comprises a phospholipid at a 1:8 molar ratio of peptide to phospholipid. In some embodiments, the formulation comprises a phospholipid at a 1:9 molar ratio of peptide to phospholipid. In some embodiments, the formulation comprises a phospholipid at a 1:10 molar ratio of peptide to phospholipid.

The present disclosure also provides methods for generating the peptide formulations described, referred to herein as peptide-phospholipids or peptide-phospholipid formulations. In some embodiments, the method for generating the peptide formulation includes mixing of the peptide with a phospholipid. In some embodiments, the peptide and phospholipid become complexed together during mixing.

The present disclosure provides the pH for generating the peptide-phospholipid formulations disclosed. In some embodiments, the peptide and phospholipid are mixed prior to pH adjustment. In some embodiments, the peptide and phospholipid mixing occurs after pH adjustment. In some embodiments, the peptide and phospholipid mixing occurs concurrently with pH adjustment. In some embodiments, the pH for mixing is a pH of 5 to 7. In some embodiments, the pH is 5, 5.5, 6, 6.5, or 7. In some embodiments, the pH during mixing facilitates peptide and phospholipid complexing and/or complex formation.

The present disclosure also provides for particular ratios of peptide to phospholipid during mixing of the peptide-phospholipid formulation. In some embodiments, the peptide and phospholipid are mixed at a 1:3 to 1:10 molar ratio of peptide to phospholipid. In some embodiments, the peptide and phospholipid are mixed at a 1:3, 1:4, 1:5, 1:6, 1:7, 1:8, 1:9, or 1:10 molar ratio of peptide to phospholipid. In some embodiments, the peptide and phospholipid are mixed at a 1:3 molar ratio of peptide to phospholipid. In some embodiments, the peptide and phospholipid are mixed at a 1:4 molar ratio of peptide to phospholipid. In some embodiments, the peptide and phospholipid are mixed at a 1:5 molar ratio of peptide to phospholipid. In some embodiments, the peptide and phospholipid are mixed at a 1:6 molar ratio of peptide to phospholipid. In some embodiments, the peptide and phospholipid are mixed at a 1:7 molar ratio of peptide to phospholipid. In some embodiments, the peptide and phospholipid are mixed at a 1:8 molar ratio of peptide to phospholipid. In some embodiments, the peptide and phospholipid are mixed at a 1:9 molar ratio of peptide to phospholipid. In some embodiments, the peptide and phospholipid are mixed at a 1:10 molar ratio of peptide to phospholipid. In some embodiments, the peptide to phospholipid ratio during mixing facilitates peptide and phospholipid complexing and/or complex formation.

Lyophilization methods for peptide solutions are well-known in the art and those of skill would readily understand how to adapt known lyophilization procedures for the peptide-phospholipid formulations of the present disclosure. In some embodiments, the disclosed peptide-phospholipid formulation is an aqueous formulation. In some embodiments, the disclosed peptide-phospholipid formulation is lyophilized. In some embodiments, after mixing the peptide and phospholipid, the method further includes a step of lyophilization.

The present disclosure also provides high-density lipoprotein-like complexes. In some embodiments, the peptide and phospholipid formulation of the present disclosure form a high-density lipoprotein-like complex. In some embodiments the present disclosure provides method for generating a high-density lipoprotein-like complex, as described herein. High-density lipoprotein-like complexes include the peptide and phospholipid mixtures described herein. In some embodiments, these high-density lipoprotein-like complexes are referred to as reconstituted high-density lipoprotein (rHDL). In some embodiments, the peptide-phospholipid formulations are referred to as rHDL particles.

The present disclosure also provides for the use of peptide-phospholipid formulations in treatment methods. In some embodiments, the peptide-phospholipid formulation effects cholesterol efflux in vivo. In some embodiments, the peptide-phospholipid formulation effects cholesterol efflux in vitro. In some embodiments, the cholesterol efflux is higher with the peptide-phospholipid formulation than with the peptide formulation where the peptide is not complexed with a phospholipid. In some embodiments, the cholesterol efflux is about 30%, about 35%, about 40%, about 45%, about 50%, about 55%, about 60%, about 65%, about 70%, about 75%, about 80%, about 90%, or about 95% or higher with the peptide-phospholipid formulation than with the peptide formulation where the peptide is not complexed with a phospholipid. In some embodiments, the cholesterol efflux is about 30% to about 95%, or about 35% to about 90%, about 40% to about 85%, about 35% to about 80%, about 35% to about 75%, about 35% to about 70%, about 40% to about 65%, about 45% to about 60%, 35% to about 55%, about 35% to about 50%, or about 40% to about 50% or higher with the peptide-phospholipid formulation than with the peptide formulation where the peptide is not complexed with a phospholipid. In some embodiments, the phospholipid is POPC. In some embodiments, the phospholipid is SM.

In some embodiments, the concentration of the peptides or peptide-phospholipid in the plasma is about 1.0 mg/mL, about 0.75 mg/mL, about 0.5 mg/mL, about 0.1 mg/mL about 0.05 mg/mL or about 0.01 mg/mL. In some embodiments, the concentration of the peptides or peptide-phospholipid in the plasma is about 0.01 mg/mL to about 1.0 mg/mL, about 0.05 mg/mL to about 1.0 mg/mL, about 0.05 mg/mL to about 0.75 mg/mL, about 0.05 mg/mL to about 0.75 mg/mL, about 0.1 mg/mL to about 0.75 mg/mL, or about 0.1 mg/mL to about 0.5 mg/mL.

The concentration of the peptide-phospholipid formulations of the disclosure can be selected primarily based on fluid volumes, viscosities, body weight in accordance with the particular mode of administration selected and the patient's needs. Concentrations, however, can be selected to provide dosages ranging from about 0.1 or 1 mg/kg/day to about 50 mg/kg/day and sometimes higher. Typical dosages range from about 3 mg/kg/day to about 3.5 mg/kg/day, or from about 3.5 mg/kg/day to about 7.2 mg/kg/day, or from about 7.2 mg/kg/day to about 11.0 mg/kg/day, or from about 11.0 mg/kg/day to about 15.0 mg/kg/day. In some embodiments, dosages range from about 10 mg/kg/day to about 50 mg/kg/day. In some embodiments, dosages range from about 20 mg to about 50 mg given orally twice daily. It will be appreciated that such dosages may be varied to optimize a therapeutic regimen in a particular subject or group of subjects. In some embodiments, the peptide-phospholipids are administered at dosage of 10 mg/kg, 20 mg/kg, 30 mg/kg, 40 mg/kg, 50 mg/kg, 60 mg/kg, 70 mg/kg, 8 mg/kg, 90 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the dosage is based on the peptide content of the formulation. In some embodiments, the dosage is based on the peptide-phospholipid content of the formulation.

Various administration regimens can be employed for the peptide-phospholipid formulations, including for treatment purposes. In some embodiments, the peptide-phospholipid formulations are administered daily, weekly or monthly. In some embodiments, the peptide-phospholipid formulations are administered 1 time, 2 times, 3 times or 4 times daily. In some embodiments, the peptide-phospholipid formulations are administered 1 time, 2 times, 3 times, 4 times, 5 times, 6 times, or 7 times or more per week. In some embodiments, administration of the peptide-phospholipid formulations occurs for 1 week, 2 weeks, 3 weeks, 4 weeks, 5 weeks, 6 weeks, 7 weeks, 8 weeks, 9 weeks, 10 weeks, 11 weeks, or 12 weeks or more. In some embodiments, administration of the peptide-phospholipid formulations occurs for 1 month, 2 months, 3 months, 4 months, 5 months, 6 months, 9 months, or 12 months or more. In some embodiments, the peptide-phospholipid formulations are administered 1 time, 2 times or 3 times weekly for 4 weeks, 5 weeks, 6 weeks, 7 weeks, or 8 weeks or more. In some embodiments, the peptide-phospholipid formulations are administered 5 times weekly for 8 weeks. In some embodiments, the peptide-phospholipid formulations are administered 4 times weekly for 6 weeks. In some embodiments, the peptide-phospholipid formulations are administered 3 times weekly for 6 weeks. In some embodiments, the peptide-phospholipid formulations are administered 3 times weekly for 4 weeks.

The present disclosure provides peptide-phospholipid formulations which interact with endogenous lipoprotein and induce remodeling of endogenous HDL in plasma. In some embodiments, the peptide-phospholipid formulations induce an increase in pre-β HDL. In some embodiments, the peptide-phospholipid formulations induce an increase in pre-β HDL after incubation with human plasma for about 5 minutes, about 10 minutes, about 15 minutes, about 20 minutes, about 25 minutes, about 30 minutes, about 35 minutes, about 40 minutes or about 45 minutes. In some embodiments, the peptide-phospholipid formulations induce an increase in pre-p HDL after incubation with human plasma for about 5 minutes to about 45 minutes, about 10 minutes to about 40 minutes, about 15 minutes to about 35 minutes, about 20 minutes to about 40 minutes or about 25 minutes to about 35 minutes.

In some embodiments, pre-β HDL increase after treatment of plasma with 5A-SM peptide-phospholipid or 5A-POPC peptide-phospholipid. In some embodiments, the increase in pre-β HDL is statistically higher for 5A-SM peptide-phospholipid as compared to 5A-POPC peptide-phospholipid.

Decreases in α-HDL and increases in pre-β HDL are indicative of a higher anti-atherosclerotic activity in vivo. In some embodiments, treatment of plasma with peptide-phospholipid 5A-SM and peptide-phospholipid 5A-POPC resulted in showed complete disappearance of α-HDL. In some embodiments, treatment of plasma with 5A-SM peptide-phospholipid and 5A-POPC peptide-phospholipid resulted in a decrease in α-HDL and an increase in pre-β HDL. In some embodiments, treatment of plasma with 5A-SM peptide-phospholipid and 5A-POPC peptide-phospholipid resulted in a complete disappearance of α-HDL and appearance of and/or increase in pre-β HDL. In some embodiments, peptide-phopholipid 5A-SM exhibits increased anti-atherosclerotic activity as compared to peptide-phopholipid 5A-POPC.

The peptide-phospholipid formulations of the present disclosure exhibit anti-inflammatory properties. In some embodiments, the peptide-phospholipids inhibit cytokine release. In some embodiments, the peptide-phospholipids inhibit cytokine release in macrophages. In some embodiments, the peptide-phospholipids inhibit cytokine release in whole blood. In some embodiments, the macrophages are peritoneal macrophages. In some embodiments, the peptide-phospholipids inhibit cytokine release. In some embodiments, the peptide-phospholipids inhibit cytokine release in a dose-dependent manner. In some embodiments, the peptide-phospholipids inhibit cytokine release after 18 hours of incubation with the peptide-phospholipid formulations. In some embodiments, the peptide-phospholipids inhibit cytokine release after administration of about 0.01 mg/mL, about 0.05 mg/mL, about 0.1 mg/mL, about 0.25 mg/mL, about 0.75 mg/mL, or about 1 mg/mL. In some embodiments, the peptide-phospholipids inhibit cytokine release after administration resulting in about 0.01 mg/mL, about 0.05 mg/mL, about 0.1 mg/mL, about 0.25 mg/mL, about 0.75 mg/mL, or about 1 mg/mL plasma concentration of the peptide-phospholipid or peptide of the present disclosure.

In some embodiments, the peptide-phospholipids disclosed exhibit dose-dependent inhibition of cytokine release. In some embodiments, 5A-SM is a more potent inhibitor of cytokine release than 5A-POPC.

In some embodiments, the cytokine whose release is inhibited is TNF-α, IL-1β, and/or IL-6 or a combination of any two or three cytokines. In some embodiments, the cytokine inhibition is greater with the 5A-SM peptide-phospholipid as compared to 5A-POPC peptide-phospholipid. In some embodiments, cytokine release is stimulated by administration of lipopolysaccharide (LPS). In some embodiments, cytokine release is stimulated by administration of lipopolysaccharide (LPS) prior to administration of the peptide-phospholipid.

The peptide-phospholipids disclosed result in cholesterol mobilization and/or esterification in vivo. The peptide-phospholipids disclosed can have affects on plasma free cholesterol (FC), cholesterol ester (CE), and/or phospholipid levels.

In some embodiments, the amount of free cholesterol increases in response to administration of the peptide-phospholipid formulations disclosed. In some embodiments, the amount of mobilized free cholesterol is proportional to the amount of peptide or peptide-phospholipid administered. In some embodiments, the amount of mobilized cholesterol reached a maximum within about 15 minutes, about 30 minutes, about 1 hour, about 1.5 hours, about 2 hours, about 3 hours, about 4 hours, about 5 hours, about 6 hours, about 7 hours, or about 8 hours post-administration of the peptide-phospholipid formulation. In some embodiments, the amount of mobilized free cholesterol reached a maximum within 1 hour to 3 hours post-administration of the peptide-phospholipid formulation. In some embodiments, the maximum free cholesterol occurs at about 3 hours post-administration of the peptide-phospholipid formulation and at a dosage of 100 mg/kg based on peptide content of peptide-phospholipid formulation.

In some embodiments, baseline levels increase by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500% as compared to baseline.

In some embodiments, the baseline level of free cholesterol is about 30±5 mg/dL. In some embodiments, administration of the peptide-phospholipid 5A-SM formulation corresponds to an increase in baseline level of free cholesterol of about 5 mg/dL, about 10 mg/dL, about 15 mg/dL, about 20 mg/dL, about 25 mg/dL, about 30 mg/dL, about 35 mg/dL, about 40 mg/dL, about 45 mg/dL, about 50 mg/dL, about 55 mg/dL, about 60 mg/dL, about 65 mg/dL, about 70 mg/dL, about 75 mg/dL, about 80 mg/dL, about 85 mg/dL, about 90 mg/dL, about 95 mg/dL, about 100 mg/dL, about 105 mg/dL, about 110 mg/dL, about 115 mg/dL, about 120 mg/dL, about 125 mg/dL, or about 130 mg/dL. In some embodiments, the increase in baseline level of free cholesterol is about 5 mg/dL to about 130 mg/dL, about 10 mg/dL to about 130 mg/dL, about 15 mg/dL to about 130 mg/dL, about 20 mg/dL to about 125 mg/dL, about 20 mg/dL to about 120 mg/dL, about 25 mg/dL to about 115 mg/dL, about 30 mg/dL to about 115 mg/dL, about 35 mg/dL to about 110 mg/dL, about 45 mg/dL to about 110 mg/dL, or about 50 mg/dL to about 110 mg/dL as compared to baseline.

In some embodiments, administration of the peptide-phospholipid 5A-SM formulation corresponds to an increase in baseline level of free cholesterol of about 105.6 mg/dL based on the peptide content of the peptide-phospholipid formulation. In some embodiments, administration of the peptide-phospholipid 5A-POPC formulation corresponds to an increase in baseline level of free cholesterol of about 53.3 mg/dL based on the peptide content of the peptide-phospholipid formulation.

In some embodiments, higher levels of cholesterol were observed after administration of peptide-phospholipid 5A-SM as compared to peptide-phospholipid 5A-POPC.

In some embodiments, administration of 5A-SM corresponds to an increase in baseline level of free cholesterol of about 107% at a peptide-phospholipid formulation dosage of about 30 mg/kg based on the peptide content of the formulation. In some embodiments, administration of peptide-phospholipid 5A-SM corresponds to an increase in baseline level of free cholesterol of about 387% at a peptide-phospholipid formulation dosage of about 100 mg/kg based on the peptide content of the formulation. In some embodiments, 30 mg/kg corresponds to an infusion of 37.5 mg/kg. In some embodiments, 100 mg/kg corresponds to an infusion of 125 mg/kg.

In some embodiments, administration of peptide-phospholipid 5A-POPC corresponds to an increase in baseline level of free cholesterol of about 53% at a peptide-phospholipid formulation dosage of about 30 mg/kg based on the peptide content of the formulation. In some embodiments, administration of peptide-phospholipid 5A-POPC corresponds to an increase in baseline level of free cholesterol of about 189% at a peptide-phospholipid formulation dosage of about 100 mg/kg based on the peptide content of the formulation. In some embodiments, 30 mg/kg corresponds to an infusion of 37.5 mg/kg. In some embodiments, 100 mg/kg corresponds to an infusion of 125 mg/kg.

In some embodiments, the plasma phospholipid level prior administration of the peptide-phospholipid formulation is about 80 mg/mL, about 85 mg/mL, about 90 mg/mL, about 95 mg/mL, about 100 mg/mL, about 110 mg/mL, about 115 mg/mL, about 120 mg/mL, about 125 mg/mL, about 130 mg/mL, about 135 mg/mL, about 140 mg/mL, about 145 mg/mL, or about 150 mg/ml. In some embodiments, the plasma phospholipid level prior administration of the peptide-phospholipid formulation is about 80 mg/mL to about 150 mg/mL, about 90 mg/mL to about 140 mg/mL, about 90 mg/mL to about 135 mg/mL, about 95 mg/mL to about 135 mg/mL, about 95 mg/mL to about 130 mg/mL, or about 100 mg/mL to about 125 mg/mL.

In some embodiments, the plasma phospholipid level after administration of the peptide-phospholipid formulation increases by about 50 mg/mL to about 100 mg/mL, about 60 mg/mL to about 90 mg/mL, or about 70 mg/mL to about 80 mg/mL. In some embodiments, the plasma phospholipid level after administration of the peptide-phospholipid formulation increases by about 50 mg/mL to about 100 mg/mL, about 60 mg/mL to about 90 mg/mL, or about 70 mg/mL to about 80 mg/mL when 30 mg/kg peptide-phospholipid based on the peptide amount is administered. In some embodiments, the plasma phospholipid level after administration of the peptide-phospholipid formulation increases by about 300 mg/mL to about 500 mg/mL, about 340 mg/mL to about 500 mg/mL, about 380 mg/mL to about 480 mg/mL, or about 400 mg/mL to about 440 mg/mL when 100 mg/kg peptide-phospholipid based on the peptide amount is administered.

In some embodiments, the plasma phospholipid increase is statistically significant about 1 hour, about 2 hours, about 3 hours, about 4 hours, about 5 hours, about 6 hours, about 7 hours, about 8 hours, about 9 hours, or about 10 hours after administration of the peptide-phospholipid formulation. In some embodiments, the plasma phospholipid levels return to pre-administration levels about 24 hours after administration of the peptide-phospholipid formulation.

The peptide-phospholipid formulations affect cholesterol esterification by lecithin-cholesterol acyltransferase (LCAT), the second step in reverse cholesterol transport. In some embodiments, administration of the peptide-phospholipid formulations results in a dose-dependent increase in cholesterol ester (CE). In some embodiments, the absolute amount of cholesterol ester increase was lower as compared to the absolute amount of free cholesterol.

The peptide-phospholipid formulations disclosed reduce the atherosclerotic burden, e.g., peptide-phospholipid formulations disclosed exhibit anti-atherosclerotic activity. “Atheroma” is a degeneration of the walls of the arteries caused by accumulated fatty deposits and scar tissue, and leading to restriction of the circulation and a risk of thrombosis. Atheroma often occurs in atherosclerosis, which is one of the three subtypes of arteriosclerosis. The three subtypes include atherosclerosis, Monckeberg's arteriosclerosis and arteriolosclerosis. In some embodiments, the baseline atheroma area is 22.5%. In some embodiments, the baseline atheroma area is 22.5%, and can increase over the treatment period. In some embodiments, the atheroma area is reduced by at least 5%, at least 7.5%, at least 10%, at least 12.5%, at least 15%, at least 17.5%, at least 20%, at least 22.5%, at least 25%, at least 27.5%, or at least 30% or more. In some embodiments, the reduction is as compared to baseline atheroma level. In some embodiments, the atheroma area is reduced at least 20% after administration of peptide-phospholipid 5A-POPC. In some embodiments, the atheroma area is reduced at least 16% after administration of peptide-phospholipid 5A-SM. In some embodiments, the atheroma area is reduced at least 28% after administration of peptide-phospholipid 5A-POPC as compared to baseline atheroma level. In some embodiments, the atheroma area is reduced at least 10% after administration of peptide-phospholipid 5A-SM as compared to baseline atheroma level.

In some embodiments, the atheroma area following placebo treatment is about 30.0% and is reduced to 24.0% following peptide-phospholipid 5A-POPC administration.

In some embodiments, the atheroma area following placebo treatment is about 30.0% and is reduced to 20.7% following peptide-phospholipid 5A-SM administration.

In some embodiments, administration of the peptide-phospholipid formulations results in plaque reduction. In some embodiments, administration of the peptide-phospholipid formulations results in aortic root lesion reduction.

The peptide-phospholipid formulations including the disclosed peptides complexed with phospholipids of the disclosure can be used alone or in combination therapy with other lipid lowering compositions or drugs used to treat the foregoing conditions. Such therapies include, but are not limited to simultaneous or sequential administration of the drugs involved. For example, in the treatment of hypercholesterolemia or atherosclerosis, the multidomain peptide or peptide analog formulations can be administered with anyone or more of the cholesterol lowering therapies currently in use, for example, bile-acid resins, niacin and statins. In some embodiments, the peptide-phospholipid formulations can be used in conjunction with statins or fibrates to treat hyperlipidemia, hypercholesterolemia and/or cardiovascular disease, such as atherosclerosis. In some embodiments, the peptide-phospholipid formulations can be used in combination with an anti-microbial agent and/or an anti-inflammatory agent.

The peptide-phospholipid formulations of the disclosure can be used to treat any disorder in animals, especially mammals (e.g., humans), for which promoting lipid efflux is beneficial, as well as the other biological properties of HDL, such as increasing endothelial cell integrity, anti-inflammation, antithrombosis, and anti-oxidation. Such conditions include, but are not limited to, hyperlipidemia (e.g., hypercholesterolemia), cardiovascular disease (e.g., atherosclerosis), restenosis (e.g., atherosclerotic plaques), peripheral vascular disease, acute coronary syndrome, reperfusion myocardial injury, and the like. They can also be used during the treatment of thrombotic and ischemic stroke and during thrombolytic treatment of occluded coronary artery disease and Alzheimer's disease.

In some embodiments, one or more of the peptide-phospholipid formulations of the disclosure are administered, in the “native” form or, if desired, in the form of salts, esters, amides, prodrugs, and/or derivatives, provided the salt, ester, amide, prodrug or derivative is suitable pharmacologically, i.e., effective in the present methods. Salts, esters, amides, prodrugs and other derivatives of the disclosed agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methodology, which typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Acid addition salts of the active agents herein are halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Basic salts include alkali metal salts, e.g., the sodium salt, and copper salts.

Preparation of esters typically involves functionalization of hydroxyl and/or carboxyl groups which may be present within the molecular structure of the drug. The esters are typically acyl-substituted derivatives of free alcohol groups, i.e., moieties that are derived from carboxylic acids of the formula RCOOH where R is alky, and in some embodiments is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The peptide-phospholipid formulations of the disclosure are useful for oral, parenteral, topical, nasal (or otherwise inhaled), rectal, or local administration, such as by aerosol or transdermally, for prophylactic and/or therapeutic treatment of one or more of the pathologies/indications described herein (e.g., atherosclerosis and/or eye disease and/or symptoms thereof). The peptide-phospholipids of the present disclosure can be administered by a variety of routes, including orally, parenterally, subcutaneously, intravascularly (e.g., intravenously, intra-arterially), or intraperitoneally. The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc. In some embodiments, administration is intravenously. In some embodiments, administration is by infusion. In some embodiments, administration is by intravenous infusion.

The peptide-phospholipid formulations of the disclosure can be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds include wetting agents, emulsifying agents, dispersing agents or preservatives that are particularly useful for preventing the growth or action of microorganisms. Various preservatives are well-known and include, for example, phenol and ascorbic acid. One skilled in the art would appreciate that the choice of pharmaceutically acceptable carrier(s), including a physiologically acceptable compound depends, for example, on the route of administration of the peptide-phospholipid formulations and on the particular physio-chemical characteristics of the peptide-phospholipid formulations.

The excipients can be sterile and generally free of undesirable matter. These compositions may be sterilized by conventional, well-known sterilization techniques.

In therapeutic applications, the peptide-phospholipid formulations of the disclosure are administered to a patient suffering from one or more symptoms of the one or more pathologies described herein, or at risk for one or more of the pathologies described herein in an amount sufficient to prevent and/or cure and/or or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient, and as described herein. In any event, the composition should provide a sufficient quantity of the active agents of the formulations of this invention to effectively treat (ameliorate one or more symptoms) of the patient.

In some embodiments, the peptide-phospholipid formulations disclosed are administered orally (e.g., via a tablet) or as an injectable in accordance with standard methods well known to those of skill in the art. In some embodiments, the peptides, can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, and functions as a primary structural element of the “patch” and provides the device with much of its flexibility. The material selected for the backing layer is typically substantially impermeable to the active agent(s) and any other materials that are present.

Other formulations for topical drug delivery include, but are not limited to, ointments and creams. Ointments are semisolid preparations which are typically based on petrolatum or other petroleum derivatives. Creams containing the selected active agent, are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

In addition, the peptide-phospholipid formulations of the disclosure can be administered via intraocular injection (e.g., intravitreal injection) in accordance with standard methods well known to those of skill in the art.

Unlike typical peptide formulations, the peptide-phospholipid formulations of this disclosure comprising D-form amino acids can be administered, even orally, without protection against proteolysis by stomach acid, etc. Nevertheless, in certain embodiments, peptide delivery can be enhanced by the use of protective excipients. This is typically accomplished either by complexing the peptide-phospholipid with a composition to render it resistant to acidic and enzymatic hydrolysis or by packaging the peptide-phospholipid formulations in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

Elevated serum half-life can be maintained by the use of sustained-release protein “packaging” systems. Such sustained release systems are well known to those of skill in the art. In one embodiment, the ProLease biodegradable microsphere delivery system for proteins and peptides (Tracy (1998) Biotechnol. Prog., 14: 108; Johnson et al. (1996) Nature Med. 2: 795; Herbert et al. (1998), Pharmaceut. Res. 15, 357) a dry powder composed of biodegradable polymeric microspheres containing the active agent in a polymer matrix that can be compounded as a dry formulation with or without other agents.

The ProLease microsphere fabrication process was specifically designed to achieve a high encapsulation efficiency while maintaining integrity of the active agent. The process consists of (i) preparation of freeze-dried drug particles from bulk by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the active agents, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the protein is maintained in the solid state in the absence of water, thus minimizing water-induced conformational mobility of the protein, preventing protein degradation reactions that include water as a reactant, and avoiding organic-aqueous interfaces where proteins may undergo denaturation. In some embodiments, the process uses solvents in which most proteins are insoluble, thus yielding high encapsulation efficiencies (e.g., greater than 95%).

In another embodiment, one or more components of the solution can be provided as a “concentrate”, e.g., in a storage container (e.g., in a premeasured volume) ready for dilution, or in a soluble capsule ready for addition to a volume of water.

The foregoing formulations and administration methods are intended to be illustrative and not limiting. It will be appreciated that, using the teaching provided herein, other suitable formulations and modes of administration can be readily devised and employed by those of skill in art.

In some embodiments, the disclosed agents are administered in conjunction with one or more additional lipids. The additional lipids can be formulated as an excipient to protect and/or enhance transport/uptake of the agents or they can be administered separately.

The additional lipids can be formed into liposomes that encapsulate the peptide-phospholipids of this invention and/or they can be complexed/admixed with the active agents and/or they can be covalently coupled to the active agents. Methods of making liposomes and encapsulating reagents are well known to those of skill in the art (see, e.g., Martin and Papahadjopoulos (1982) J. Biol. Chem., 257: 286-288; Papahadjopoulos et al. (1991) Proc. Natl. Acad. Sci. USA, 88: 11460-11464; Huang et al. (1992) Cancer Res., 52:6774-6781; Lasic et al. (1992) FEBS Lett., 312: 255-258., and the like).

In some embodiments, the one or more additional lipid is a phospholipid. In some embodiments, the additional phospholipid(s) for use in the disclosed methods have fatty acids ranging from about 4 carbons to about 24 carbons in the sn-1 and sn-2 positions. In some embodiments, the fatty acids are saturated. In some embodiments, the fatty acids can be unsaturated.

The fatty acids in these positions can be the same or different. In some embodiments, the additional phospholipid has phosphorylcholine at the sn-3 position.

In some embodiments the peptide-phospholipids of the disclosure are contained within biocompatible matrices (e.g. biocompatible polymers such as urethane, silicone, and the like). Suitable biocompatible materials are described, for example, in U.S. Patent Publications 2005/0084515, 2005/00791991, 2005/0070996, and the like which are incorporated herein by reference in their entireties for all purposes. In various embodiments the polymers include, but are not limited to silicone-urethane copolymer, a polyurethane, a phenoxy, ethylene vinyl acetate, polycaprolactone, poly(lactide-co-glycolide), polylactide, polysulfone, elastin, fibrin, collagen, chondroitin sulfate, a biocompatible polymer, a biostable polymer, a biodegradable polymer.

Thus, in some embodiments this invention provides a stent for delivering drugs to a vessel in a body. The stent typically comprises stent framework including a plurality of reservoirs formed therein. The reservoirs typically include a peptide-phospholipid and/or peptide-phospholipid-containing polymer positioned in the reservoir and/or coated on the surface of the stent. In various embodiments the stent is a metallic base or a polymeric base. Stent materials include, but are not limited to stainless steel, nitinol, tantalum, MP35N alloy, platinum, titanium, a suitable biocompatible alloy, a suitable biocompatible polymer, and/or a combination thereof.

In some embodiments where the stent comprises pores (e.g., reservoirs), the pores can include micropores (e.g., having a diameter that ranges from about 10 to about 50 μm, in some embodiments about 20 μm or less). In various embodiments the micropores have a depth in the range of about 10 μm to about 50 μm. In various embodiments the micropores extend through the stent framework having an opening on an interior surface of the stent and an opening on an exterior surface of the stent. In certain embodiments the stent can, optionally comprise a cap layer disposed on the interior surface of the stent framework, the cap layer covering at least a portion of the through-holes and providing a barrier characteristic to control an elution rate of the active agent(s) in the polymer from the interior surface of the stent framework. In various embodiments the reservoirs comprise channels along an exterior surface of the stent framework. The stent can optionally have multiple layers of polymer where different layers of polymer carry different active agent(s) and/or other drugs.

In certain embodiments the stent comprises: an adhesion layer positioned between the stent framework and the polymer. Suitable adhesion layers include, but are not limited to a polyurethane, a phenoxy, poly(lactide-co-glycolide)-, polylactide, polysulfone, polycaprolactone, an adhesion promoter, and/or a combination thereof.

In addition to stents, the peptide-phospholipid formulations can be coated on or contained within essentially any implantable medical device configured for implantation in a extravascular and/or intravascular location.

Also provided are methods of manufacturing a drug-polymer stent, comprising the peptide-phospholipid formulations disclosed herein. The methods involve providing a stent framework; cutting a plurality of reservoirs in the stent framework, e.g., using a high power laser; applying one or more of the active agents and/or a drug polymer to at least one reservoir; drying the drug polymer; applying a polymer layer to the dried drug polymer; and drying the polymer layer. The active agent(s) and/or polymer(s) can be applied by any convenient method including but not limited to spraying, dipping, painting, brushing and dispensing.

Also provided are methods of treating a vascular condition and/or a condition characterized by an inflammatory response and/or a condition characterized by the formation of oxidized reactive species. The methods typically involve positioning a stent or other implantable device as described above within the body (e.g. within a vessel of a body) and eluting at least one peptide-phospholipid or peptide portion of the peptide-phospholipid from at least one surface of the implant.

In certain embodiments, one or more peptide-phospholipids as described herein are administered alone or in combination with other therapeutics as described herein in implantable (e.g., subcutaneous) matrices.

A drawback with standard drug dosing is that typical delivery of drugs results in a quick burst of medication at the time of dosing, followed by a rapid loss of the drug from the body. Most of the side effects of a drug occur during the burst phase of its release into the bloodstream. Secondly, the time the drug is in the bloodstream at therapeutic levels is very short, and as such most is used and cleared during the short burst.

Drugs (e.g., the peptide-phospholipids described herein) imbedded in various matrix materials for sustained release can provide some solution to these problems. Peptide-phospholipid embedded, for example, in polymer beads or in polymer wafers have several advantages. First, most systems allow slow release of the drug, thus creating a continuous dosing of the body with small levels of the peptide-phospholipids. This typically prevents side effects associated with high burst levels of normal injected or pill based drugs. Secondly, since these polymers can be made to release over hours to months, the therapeutic span of the peptide-phospholipid is markedly increased. Often, by mixing different ratios of the same polymer components, polymers of different degradation rates can be made, allowing remarkable flexibility depending on the agent being used. A long rate of peptide-phospholipids release can be beneficial for people who might have trouble staying on regular dosage, such as the elderly, but is also an ease of use improvement that everyone can appreciate. Most polymers can be made to degrade and be cleared by the body over time, so they will not remain in the body after the therapeutic interval.

Another advantage of polymer based peptide-phospholipid delivery is that the polymers often can stabilize or solubilize proteins, peptides, and other large molecules that would otherwise be unusable as medications. Finally, many drug/polymer mixes can be placed directly in the disease area, allowing specific targeting of the medication where it is needed without losing drug to the “first pass” effect. This is certainly effective for treating the brain, which is often deprived of medicines that cannot penetrate the blood/brain barrier.

A number of implantable matrix (sustained release) systems are known to those of skill and can readily be adapted for use with one or more of the peptide-phospholipids described herein. Suitable sustained release systems include, but are not limited to Re-Gel®, SQ2Gel®, and Oligosphere® by MacroMed, ProLease® and Medisorb® by Alkermes, Paclimer® and Gliadel® Wafer by Guilford pharmaceuticals, the Duros implant by Alza, acoustic bioSpheres by Point Biomedical, the Intelsite capsule by Scintipharma, Inc., and the like.

Other “specialty” delivery systems include, but are not limited to additional lipid based oral mist that allows absorption of peptide-phospholipids and/or the peptide portion of the peptide-phospholipid across the oral mucosa, developed by Generex Biotechnology, the oral transmucosal system (OTS™) by Anesta Corp., the inhalable dry powder and PulmoSpheres technology by Inhale Therapeutics, the AERx® Pulmonary Drug Delivery System by Aradigm, and/or the AIR mechanism by Alkermes.

Another approach to delivery developed by Alkermes is a system targeted for elderly and pediatric use, two populations for which taking pills is often difficult is known as Drug Sipping Technology (DST). The medication is placed in a drinking straw device, prevented from falling out by filters on either end of it. The patient merely has to drink clear liquid (water, juice, soda) through the straw. The drug dissolves in the liquid as it is pulled through and is ingested by the patient. The filter rises to the top of the straw when all of the medication is taken. This method has the advantage in that it is easy to use, the liquid often masks the medication's taste, and the drug is pre-dissolved for more efficient absorption.

It is noted that these uses and delivery systems are intended to be illustrative and not limiting. Using the teachings provided herein, other uses and delivery systems will be known to those of skill in the art.

The peptide-phospholipids of the disclosure can be co-administered with other agents, such as niclosamide, which have been shown to further prevent proteolysis and enhance absorption of amphipathic peptides (Navab et al. (2009) ASBMB, 50:1538-1547). In some embodiments, the other or additioanl agent is administered before, after or concurrently with the peptide-phosholipid formulations disclosed herein.

In various embodiments, the use of combinations of two or more agents described is contemplated in the treatment of the various pathologies/indications described herein. The use of combinations of peptide-phospholipids as well as combinations with additional agents can alter pharmacological activity and bioavailability.

In certain embodiments this disclosure contemplates combinations of, for example, these two or more peptide-phospholipids to reduce production expense, and/or to optimize dosage regimen, therapeutic profile, and the like. In certain embodiments combinations of the peptide-phospholipids described herein can be simply co-administered and/or added together to form a single pharmaceutical formulation. In certain embodiments the various peptide-phospholipids can be complexed together (e.g. via hydrogen bonding) to form peptide-phospholipids complexes that are more effective than the parent agents.

Additional pharmacologically active materials (i.e., drugs) can be delivered in conjunction with one or more of the peptide-phospholipids described herein. In certain embodiments, such agents include, but are not limited to agents that reduce the risk of atherosclerotic events and/or complications thereof. Such agents include, but are not limited to beta blockers, beta blockers and thiazide diuretic combinations, statins, aspirin, ACE inhibitors, ACE receptor inhibitors (ARBs), and the like.

Thus, in some embodiments this invention provides methods for enhancing the activity of statins. In some embodiments, the peptide-phospholipid formulations can be administered with one or more statins. The methods generally involve administering one or more of the peptide-phospholipids described herein, as described herein in conjunction with one or more statins. The peptide-phospholipids can achieve synergistic action between the statin and the agent(s) to ameliorate one or more symptoms of atherosclerosis. In this context statins can be administered at significantly lower dosages thereby avoiding various harmful side effects (e.g., muscle wasting) associated with high dosage statin use and/or the anti-inflammatory properties of statins at any given dose are significantly enhanced.

Suitable statins include, but are not limited to pravastatin (Pravachol™/Bristol-Myers Squibb), simvastatin (Zocor™/Merck), and lovastatin (Mevacor™/Merck).

In some embodiments, the peptide-phospholipids described herein are administered in conjunction with one or more beta blockers. Suitable beta blockers include cardioselective (selective beta 1 blockers), e.g., acebutolol (Sectral™), atenolol (Tenormin™), betaxolol (Kerlone™), bisoprolol (Zebeta™), metoprolol (Lopressor™). Suitable non-selective blockers include without limitataion carteolol (Cartrol™), nadolol (Corgard™), penbutolol (Levatol™), pindolol (Visken™), propranolol (Inderal™), timolol (Blockadren™) and labetalol (Normodyne™, Trandate™).

Suitable beta blocker thiazide diuretic combinations include but are not limited to Lopressor HCT, ZIAC, Tenoretic, Corzide, Timolide, Inderal LA 40/25, Inderide, and Normozide.

Suitable ACE inhibitors include, but are not limited to captopril (e.g. Capotez™ by Squibb), benazepril (e.g., Lotensin™ by Novartis), enalapril (e.g., Vasotec™ by Merck), fosinopril (e.g., Monopril™ by Bristol-Myers), lisinopril (e.g. Prinivil™ by Merck or Zestril™ by Astra-Zeneca), quinapril (e.g. Accupril™ by Parke-Davis), ramipril (e.g., Altace™ by Hoechst Marion Roussel, King Pharmaceuticals), imidapril, perindopril erbumine (e.g., Aceon™ by Rhone-Polenc Rorer) and trandolapril (e.g., Mavik™ by Knoll Pharmaceutical). Suitable ARBS (Ace Receptor Blockers) include but are not limited to losartan (e.g. Cozaar™ by Merck), irbesartan (e.g., Avapro™ by Sanofi), candesartan (e.g., Atacand™ by Astra Merck), and valsartan (e.g., Diovan™ by Novartis).

In various embodiments, one or more peptide-phospholipids described herein are administered with one or more of the drugs identified above and/or below.

Thus, in certain embodiments one or more peptide-phospholipids are administered in conjunction with cholesteryl ester transfer protein (CETP) inhibitors (e.g., torcetrapib, JTT-705. CP-529414) and/or acyl-CoA:cholesterol O-acyltransferase (ACAT) inhibitors (e.g., Avasimibe (CI-1011), CP 113818, F-1394, and the like), and/or immunomodulators (e.g., FTY720 (sphingosine-1-phosphate receptor agonist), Thalomid (thalidomide), Imuran (azathioprine), Copaxone (glatiramer acetate), Certican® (everolimus), Neoral® (cyclosporine), and/or dipeptidyl-peptidase-4 (DPP4) inhibitors (e.g., 2-Pyrrolidinecarbonitrile, 1-[[[2-[(5-cyano-2-pyridinyl)amino]ethyl]amino]acetyl], see also U.S. Patent Publication 2005-0070530), and/or calcium channel blockers (e.g., Adalat, Adalat CC, Calan, Calan SR, Cardene, Cardizem, Cardizem CD, Cardizem SR, Dilacor-XR, DynaCirc, Isoptin, Isoptin SR, Nimotop, Norvasc, Plendil, Procardia, Procardia XL, Vascor, Verelan), and/or peroxisome proliferator-activated receptor (PPAR) agonists for, e.g., α, γ, receptors (e.g., Azelaoyl PAF, 2-Bromohekadecanoic acid, Ciglitizone, Clofibrate, 15-Deoxy-δ^{12,14-}prostaglandin J₂, Fenofibrate, Fmoc-Leu-OH, GW1929, GW7647, 8(S)-Hydroxy-(5Z,9E,11Z,14Z)-eicosatetraenoic acid (8(S)-HETE), Leukotriene B₄, LY-171,883 (Tomelukast), Prostaglandin A₂, Prostaglandin J₂, Tetradecylthioacetic acid (TTA), Troglitazone (CS-045), and WY-14643 (Pirinixic acid).

In certain embodiments one or more of the peptide-phospholipids are administered in conjunction with fibrates (e.g., clofibrate (atromid), gemfibrozil (lopid), fenofibrate (tricor), etc.), bile acid sequestrants (e.g., cholestyramine, colestipol, etc.), cholesterol absorption blockers (e.g., ezetimibe (Zetia), etc.), Vytorin (ezetimibe/simvastatin combination), and/or steroids, warfarin, and/or aspirin and/or angiotensin II receptor antagonists (e.g., losartan (Cozaar), valsartan (Diovan), irbesartan (Avapro), candesartan (Atacand) and telmisartan (Micardis).

In another embodiment this invention provides kits for amelioration of one or more symptoms of atherosclerosis or for the prophylactic treatment of a subject (human or animal) at risk for atherosclerosis and/or the treatment or prophylaxis of one or more of the conditions described herein.

The kits can comprise a container containing one or more of the peptide-phospholipids and/or peptide-phospholipids formulations disclosed herein. The peptide-phospholipids can be provided in a unit dosage formulation (e.g. suppository, tablet, caplet, patch, etc.) and/or may be optionally combined with one or more pharmaceutically acceptable excipients.

The kit can, optionally, further comprise one or more other agents used in the treatment of the condition/pathology of interest. Such agents include, but are not limited to, beta blockers, vasodilators, aspirin, statins, ACE inhibitors or ace receptor inhibitors (ARBs) as described above.

In addition, the kits optionally include labeling and/or instructional materials providing directions (i.e., protocols) for the practice of the methods or use of the “therapeutics” or “prophylactics” of this invention. In some embodiments, instructional materials describe the use of one or more peptide-phospholipids disclosed herein to mitigate one or more symptoms of atherosclerosis (or other pathologies described herein) and/or to prevent the onset or increase of one or more of such symptoms in an individual at risk for atherosclerosis (or other pathologies described herein). The instructional materials may also, optionally, teach dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

### Example 1

### The Effect of Phospholipid Composition of Reconstituted HDL on its Cholesterol Efflux and Anti-Inflammatory Properties

### Abstract

The goal of this study was to understand how the reconstituted IDL) phospholipid composition affects its cholesterol efflux, and anti-inflammatory properties. 5A, an apolipoprotein A-I mimetic peptide, was combined with either sphingomyelin (SM) or palmitoyl-2-oleoyl-sn-glycero-3-phosphocholine (POPC) to form 8-10 nm rHDL disks. Both lipid formulations exhibited similar in vitro cholesterol efflux by ABCA1, but 5A-SM exhibited higher ABCG1 and SR-BI mediated efflux relative to 5A-POPC. Injection of both rHDLs in rats resulted in dose-dependent mobilization of plasma cholesterol, although the relative amount of mobilized cholesterol was 3-fold higher for the same doses of 5A-SM than for 5A-POPC. The plasma from animals dosed with 5A-SM-HDL showed greater ABCA1, ABCG1 and SR-BI efflux capacities relative to 5A-POPC-HDL dosed animals. Formation of pre-β HDL was observed following incubation of rHDL with human plasma in vitro, with 5A-SM inducing higher extent of pre-β formation relative to 5A-POPC. Both rHDLs exhibited anti-inflammatory properties, but 5A-SM-HDL showed slightly higher inhibition of TNF-α, IL-6 and IL-10 release from macrophages than did 5A-POPC-HDL. The type of phospholipid use to reconstitute 5A peptide has significant influence on cholesterol efflux in vitro, cholesterol mobilization in vivo, pre-β HDL formation and on the anti-inflammatory properties of rHDLs, which has important implications for the development of rHDL for the treatment of cardiovascular disease.

### 1. Introduction

Infusions of cholesterol-free reconstituted high-density lipoprotein (rHDL) particles have been shown to rapidly reverse atherosclerosis in a wide variety of animal models and in clinical trials of acute coronary syndrome (ACS) patients (Remaley, A. T., Amar, A., Sviridov, D. (2009) Expert Rev Cardiovasc Ther; 6(9): 1203-15; Newton, R. S., Krause, B. R. (2002) Atheroscler Suppl, 3(4): 31-8; Badimon, J. J., Badimon, L., Fuster, V. (1990) J Clin Invest, 85(4): 1234-41; Nissen, S. E., et al. (2003) JAMA, 290: 2292-2300; and Tardif, J. C., et al. (2007) JAMA; 297: 1675-1682). While a significant emphasis has been placed on investigating the HDL protein component i.e., Apolipoprotein A-I (ApoA-I), ApoA-I mutants and mimetic peptides, the importance of HDL phospholipid composition not been systematically investigated. Lipid represents 50 to 80% of the total HDL mass and is known to affect particle stability in vivo, cholesterol efflux from macrophages, ability to interact with lecithin-cholesterol acyltransferase (LCAT) and cholesterol elimination (Rye, K. A., Hime, N. J., Barter, P. J. (1996) J. Biol Chem, 271: 4243-4250; Davidson, W. S., et al. (1995) J. Biol. Chem., 270: 5882-5890; Marmillot, P., Patel, S., Lakshman, M. R. (2007) Metabolism, 56(2): 251-9; Ma, C. I., et al. (2012) Biochem Cell Biol, 90(5): 636-45; Sparks, D. L., et al. (1995) J. Biol. Chem., 270: 26910-26917; and Parks, J. S., et al. (2000) J Lipid Res, 41: 546-553). Lipid composition also largely defines the size, net charge and rigidity of the rHDL particles—all are important factors in the pharmacokinetic and pharmacodynamics properties of rHDL. The investigation of the effects of lipid composition on the resulting rHDL properties in vitro and in vivo is the focus of this article. By better defining the lipid effect of rHDL, the goal was to be able to favorably alter the potency and safety of rHDL, and ultimately advance clinical translation of these potentially life-changing nanomedicines.

The phospholipid composition of endogenous HDL contains phosphatidylcholines (PC), sphingomyelin (SM) and small amounts of lysophosphatidylcholine (LPC), phosphatidylethanolamine (PE), phosphatidylinositol (PI) and other lipids (Kontush, A., Lhomme, M., Chapman, M. J. (2013) J Lipid Res, 54: 2950-2962; Camont, L., et al. (2013) Arterioscler Thromb Vase Biol, 33(12): 2715-2723; and Kontush A., et al. (2007) Arterioscler Thromb Vase Biol., 27: 1843-1849). The differences in lipid composition of various subclasses of HDL and how they relate to disease have been gaining attention in recent years (Kontush, A., Lhomme, M., Chapman, M. J. (2013) J Lipid Res, 54: 2950-2962; Camont, L., et al. (2013) Arterioscler Thromb Vase Biol, 33(12): 2715-2723; and Kontush A., et al. (2007) Arterioscler Thromb Vasc Biol., 27: 1843-1849). However, a single source of lipid is used for preparation of reconstituted HDL for most academic research and clinical development (Remaley, A. T., Amar, A., Sviridov, D. (2009) Expert Rev Gardiovasc Ther; 6(9): 1203-15; Newton, R. S., Krause, B. R. (2002) Atheroscler Suppl, 3(4): 31-8; Badimon, J. J., Badimon, L., Fuster, V. (1990) J Clin Invest, 85(4): 1234-41; Nissen, S. E., et al. (2003) JAMA, 290: 2292-2300; Tardif, J. C., et al. (2007) JAMA; 297: 1675-1682; Carlson, L. (1995) Nutr Metab Cardiovasc Dis, 5: 85-91; Nanjee, M. N., et al. (1999) Arterioscler Thromb Vasc Biol, 19(4): 979-89; Marchesi, M., et al. (2004) J Pharmacol Exp Ther, 311(3): 1023-31; and Hippalgaonkar, K., et al. (2010) AAPS PharmSciTech, 11(4): 1526-1540). The first lipid used to prepare rHDLs for human testing was soybean phosphatidylcholine (soyPC), because this lipid was used in parenteral nutrition products and was readily commercially available in infusion grade (Hippalgaonkar, K., et al. (2010) AAPS PharmSciTech, 11(4): 1526-1540). Thus recombinant proapolipoprotein A-I (a product of UCB) and blood purified ApoA-I (CSL-111, a products of CSL Behring) were reconstituted with soyPC (Carlson, L. (1995) Nutr Metab Cardiovasc Dis, 5: 85-91; Nanjee, M. N., et al. (1999) Arterioscler Thromb Vasc Biol, 19(4): 979-89; Marchesi, M., et al. (2004) J Pharmacol Exp Ther, 311(3): 1023-31). As more infusion grade chemically defined lipids became commercially available, the phospholipid composition used in the preparation of rHDL diversified. A 1-palmitoyl-2-oleoyl-sn-glycero-3-phosphocholine (POPC) was first used in ETC-216 (rApoA-I-Milano), and then a mixture of 1,2-dipalmitoyl-sn-glycero-3-phosphocholine (DPPC) and SM was used for ETC-642, a peptide-based rHDL (Marchesi, M., et al. (2004) J Pharmacol Exp Ther, 311(3): 1023-31; and Di Bartoloa, et al. (2011) Atherosclerosis, 217: 395-400). Most recently, SM was used in CER-001, another newly developed rApoA-I-based rHDL (International Application No. WO/2012/109162). Both sphingomyelin and saturated fatty acid containing lipids like DPPC have superior cholesterol binding capacity relative to unsaturated POPC and soyPC (Ohvo-Rekilä, H., Ramstedt et al. (2002) Prog Lipid Res, 41(1): 66-97; and Ramstedt, B., Slotte, J. P. (1999) Biophys. J. 76: 908-915). In fact, SM has the strongest cholesterol binding affinity governed by Van der Waals' interaction due to the mismatched fatty acid lengths (Ohvo-Rekilä, H., Ramstedt et al. (2002) Prog Lipid Res, 41(1): 66-97). SM is also known to form lipid-raft sections in cellular membrane that contain up to 50% of cholesterol (Silva. L. C., Futerman, A. H., Prieto, M. (2009) Biophys. J. 96(8): 3210-22). The rHDLs made from saturated lipids exhibited greater cholesterol efflux from macrophages in vitro (Marmillot, P., et al. (2007) Metabolism, 56(2): 251-9; Ma, C. I., et al. (2012) Biochem Cell Biol, 90(5): 636-45; and Sparks, D. L., et al. (1995) J. Biol. Chem., 270: 26910-26917) and cholesterol mobilization in vivo (International Patent Publication No. WO 2003/096983). However, unsaturation enhances rHDL interaction with LCAT (Parks, J. S., et al. (2000) J Lipid Res, 41: 546-553), whereas SM was reported to inhibit cholesterol esterification in vitro (Rye, K. A., et al. (1996) J. Biol Chem, 271: 4243-4250).

In this study, it was hypothesized that higher cholesterol binding affinity of SM relative to POPC would translate into greater cholesterol efflux both in vitro and in vivo. The ApoA-I mimetic, 5A, a 37-amino acid-long bi-helical peptide (25), was used to form SM and POPC-based HDL particles. The 5A based HDL has several similar features to HDL prepared from the full-length ApoA-I, namely, an equivalent ability to promote cholesterol efflux in vitro and in vivo (Sethi, A. A., et al. (2008) J Biol Chem, 283:32273-32282; and Amar, M. J. A., et al. (2010) J Pharmacol Exp Ther, 334: 634-641), and similar abilities in reducing the development of atherosclerosis (Amar, M. J. A., et al. (2010) J Pharmacol Exp Ther, 334: 634-641) and suppressing inflammation (Tabet, F., et al. (2010) Arterioscler Thromb Vasc Biol, 30: 246-252). In this study, differences in cholesterol efflux and inflammatory cytokine release inhibition were examined for 5A-POPC and 5A-SM. In addition, the ability to mobilize and esterify cholesterol in vivo depending on the dose and lipid composition of infused rHDL was evaluated in rats.

### 2. Materials and Methods

### 2.2 Materials

5A (DWLKAFYDKVAEKLKEAFPDWAKAAYDKAAEKAKEAA; SEQ ID NO:1) was synthesized by Genscript (Piscataway, N.J.), using solid-phase Fmoc (9-fluorenylmethyl carbamate) protection chemistry and was purified with reverse phase chromatography. Peptide purity was >95% as determined by HPLC. Egg sphingomyelin and 1-palmitoyl-2-oleoyl-sn-glycero-3-phosphocholine (POPC) were purchased from Avanti Polar Lipids (Alabaster, Ala.) and Nippon Oil and Fat (Osaka, Japan). All other materials were obtained from commercial sources.

### 2.2 Preparation and Characterization of 5A Phospholipid Complexes

High-density lipoprotein-like 5A-POPC and 5A-SM complexes were prepared by a co-lyophilization procedure (25, 26). Peptide and phospholipids were dissolved in glacial acetic acid and mixed at 1:1.25 w/w ratio (approximately 1:7 molar ratio) and lyophilized. The powder was hydrated with bicarbonate buffered saline and cycled between 50° C. and room temperature to facilitate 5A-lipid binding. The resulting HDL complexes were analyzed by gel permeation chromatography, with UV detection at 220 nm, using Tosoh TSK gel G3000SWx1 column (Tosoh Bioscience, King of Prussia, Pa.). The HDL hydrodynamic diameters were determined by dynamic light scattering (DLS), using a Zetasizer Nano, Malvern Instruments (Westborough, Mass.). The number and intensity average values were reported. The alpha-helical contents of free and lipid bound 5A peptide were determined by Jasco J715 (Jasco, Easton, Md.) circular dichroism spectropolarimeter. Samples at 0.1 mg/mL concentration were loaded into a quartz cuvette (d=0.2-cm path length), and CD spectra from 185 to 240 nm were recorded at 24° C. Data were normalized by calculating the mean residue ellipticity (θ).

Transmission electron microscopy (TEM) images were obtained using JEM 1200EX electron microscope (JEOL USA) equipped with an AMT XR-60 digital camera (Advanced Microscopy Techniques Corp). Images were acquired at magnifications from 60,000 to 150,000-fold. HDL samples, 3 μL aliquots, were deposited on carbon film-coated 400 mesh copper grids (Electron Microscopy Sciences) and dried for 1 min. The samples were negatively stained with 1% uranyl acetate solution; the grids were blotted with tissue and dried before TEM observation.

2.3 Cholesterol Efflux Assay in vitro

Cholesterol efflux studies were performed, as described previously (25, 26). Briefly, BHK-mock and BHK stably transfected cells with human ABCA1 cDNA, ABCG1 cDNA and SR-BI cDNA were labeled for 24 h with 1 μCi/ml of ³H-cholesterol in minimum essential medium medium plus 10% fetal calf serum. Transporters were induced with 10 nM mifepristone in Dulbecco's modified Eagle's medium (DMEM), containing 0.2 mg/ml of fatty acid-free bovine serum albumin for 18 h. Following induction of the transporters, the cells were washed and test articles were added at 0.1, 2.5, 5, 10, 20 and 40 μM in DMEM-BSA containing mifepristone. The cholesterol efflux by BSA, free 5A peptide, purified HDL, and ApoA-I were used as quality control standards. After 18 h of incubation, media were collected and filtered using 24-well, 25-μm pore size plate filter (Whatman—GE Healthcare, Pittsburgh, Pa.) and cells were lysed in 0.4 ml of 0.1% SDS and 0.1 N NaOH. Radioactive counts in media and cell fractions were measured by liquid scintillation counting, and percent cholesterol effluxed was calculated by dividing media count by the sum of media and cell counts. The percent of cholesterol efflux by blank BSA was subtracted from the results to account for background efflux.

Cholesterol efflux by plasma samples was performed using a slightly modified procedure. C57BL/6 mice were given intravenous 60 mg/kg dose of 5A-POPC, 5A-SM or saline into the tail vein (n=3/dose/time point). The plasma samples were collected at pre-dose, 0.5, 1, 2, 4 and 24 h post dose and ApoB containing lipoproteins were removed from plasma by precipitation with 5.7% solution of prolyethelene glycol 8000 Da. BHK cells were grown, induced and loaded with ³H-cholesterol as described above. HDL fraction of plasma samples were added to the media at 1% (v/v) and efflux experiment was performed for 18 h. Blank media, 5A lipid-free peptide, plasma purified HDL and ApoAI were used as controls. The cells were lysed by addition of 0.5 mL of 1% sodium cholate and 0.05% SDS solution.

### 2.4 Plasma HDL Remodeling

Remodeling of rHDL in plasma was assessed by addition of 50 μL of 5, 1 or 0.5 mg/ml of 5A-SM or 5A-POPC to 450 μL of pooled human plasma. The final concentrations of peptide in plasma were 0.5, 0.1 and 0.05 mg/mL respectively. Plasma incubation with the same volume of PBS was used as a control. Samples were incubated at 37° C. for 1 hour under shaking at 300 rpm prior to electrophoresis. The various sub-classes of HDL were separated by size and charge by one- or two-dimension native page gel electrophoresis and visualized by western blot using anti-ApoA-I antibody.

To visualize native HDL particles separated by size, samples were subjected to electrophoresis using 10-well Tris-Borate-EDTA gradient (3-25%) acrylamide mingels (Jule, Inc., Milford, Conn.) (28). For each well, 5 μl of plasma incubated with or without rHDL was mixed with 5 μl of 2×TBE sample buffer and 6 μl of the resulting mixtures were loaded per well. Gels were run at 200 V until the sample dye was 2.5 cm away from the bottom of the gel. Proteins were visualized by western blot by transfer onto PVDF membrane and incubation overnight with anti-human apoA-I-HRP conjugated antibody (Meridian Life Science, Memphis, Tenn.). Antibody solution was prepared by mixing 3 μl of antibody with 40 mL of antibody dilution buffer. Images were acquired on an Alpha Innotech Chemi Imager 5500 and the Alpha Ease FC program was used for spot densitometry.

To visualize native HDL particles separated by charge and size, samples were subjected to native-native 2D gel electrophoresis (Freeman, L. A. (2013) Western blots. Methods Mol Biol., 1027:369-85). For each sample, 2 μl plasma incubated with rHDLs or PBS control, 4 μl sample buffer and 8 μl Tris tricine buffer were combined. A 10.0 μl aliquot was added per well of the first-dimension gel (0.7% agarose), followed by 2^{nd}-dimension electrophoresis on a 3-25% acrylamide gradient TBE mini-gels (Jule). The western blot analysis and visualization was performed as described above. The classification of various HDL sub-classes was performed in accordance with Asztalos et al (30).

### 2.5 Inhibition of Cytokine Release Testing

Inhibition of cytokine release from murine peritoneal macrophages following lipopolysaccharides (LPS) stimulation was determined for 5A-SM, 5A-POPC, free 5A peptide and PBS controls. Mouse peritoneal lavage was performed with 5 mL of sterile PBS. Peritoneal macrophages were washed, collected by centrifugation and reconstituted in DMEM containing 10% fetal bovine serum (FBS). Cells were plated, attached, washed twice with PBS, stripped by trypsin and counted. Macrophages were seeded at 100,000 cells per well, incubated for 2 h, following by addition of test articles at 0.01, 0.1 and 1 mg/mL for 18 h. The cells were washed again, treated with 1 μg/mL of LPS for 2 h, washed again and incubated for 6 h. The media was collected and the levels of TNF-α were determined by for BioLegend ELISA kits (San Diego, Calif.) as per the manufacturer's instructions.

Determination of cytokine release inhibition in whole human blood was performed by pretreating heparinized human blood for 1 h with 5A-SM, 5A-POPC, free 5A peptide controls at 0, 0.01, and 0.1 mg/mL peptide concentrations. Cytokine release was stimulated by addition of 1 μg/mL of phytohemaglutinin-M (PHA) followed by an overnight incubation. The cells were collected by centrifugation and the levels of IL-113, IL-6 and TNF-α were measured by Human ProInflammatory-4 I tissue culture kit using SECTOR® Imager 2400 from Meso Scale Discovery (Rockville, Md. Rockville, Md.).

2.6 Cholesterol Mobilization and Esterification in vivo,

Sprague-Dawley rats (8 weeks old) were purchased from Charles River Breeding Laboratories (Portage, Mich.) and were fed a regular rodent chow diet. To determine the dose response, 5A-SM and 5A-POPC were dosed intravenously via tail veil at 10, 30 and 100 mg/kg doses based on the peptide amount. The plasma samples were collected pre-dose and at 1, 2, 4, 6, 8, 12, and 24 h after the infusion and the levels of plasma total cholesterol (TC) and unesterified or free cholesterol (FC) were determined by enzymatic analysis using commercially available kits (Wako Chemicals, Richmond, Va.). Cholesterol ester level was calculated as a difference between TC and FC levels at each time point.

### 3. Results

3.1 Preparation and Characterization of rHDLs

Reconstituted HDL (rHDL) particles were prepared by combining 5A peptide with either SM or POPC, using a co-lyophilization procedure. The peptide and lipid powders were dissolved in organic solvent and freeze-dried to obtain homogeneous powder. The powders were hydrated with bicarbonate buffered saline and heated to 50° C. to facilitate dissolution and phospholipid peptide binding. Preliminary studies were performed to determine the optimal weight ratio of peptide to phospholipids for formation of homogeneous rHDL complexes. The ratio of 1:1.25 wt/wt or 1:7 mole:mole of peptide to phospholipid was selected. Higher ratios of lipid to peptide resulted in greater rHDL heterogeneity and the presence of liposome impurities. Lower amounts of lipid resulted in the presence of some lipid free peptide. The optimum weight ratio of peptide to phospholipid ranged between 1:1.25 and 1:1.5 and resulted in homogenous rHDL particle size distribution. The 1:1.25 ratio was chosen as the preferred formulation.

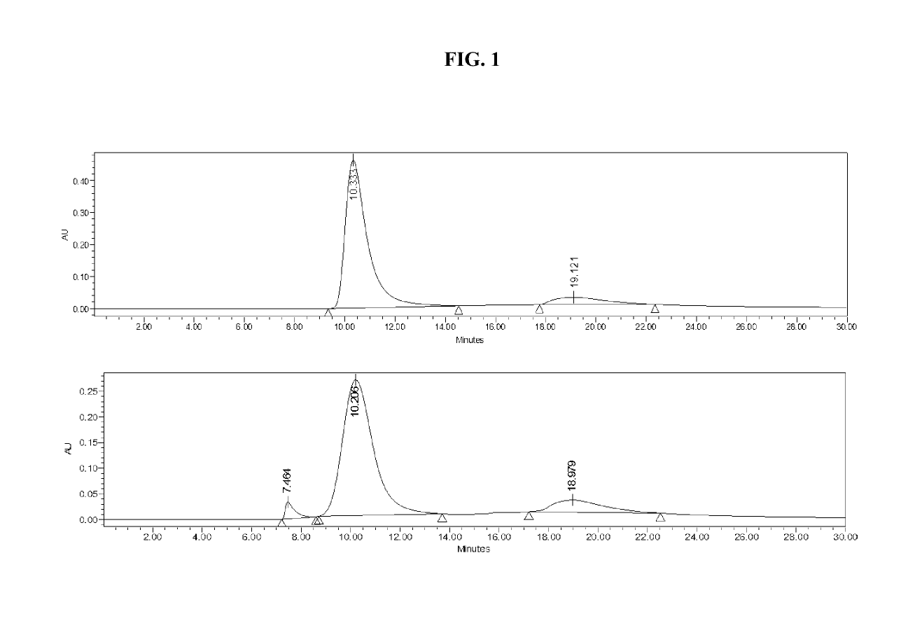

The analysis of the purity of rHDL complexes was performed by gel permeation chromatography (GPC) shown in FIG. 1. The results, summarized in Table 1, indicate formation of rHDL (elutes at 10 min), with a presence of small impurity from a liposome peak (7.5 min retention time) and free 5A peptide (20 min retention time). The POPC-HDL contained slightly higher percent of unbound 5A and liposome peaks relative to the SM-HDL preparation. The HDL peak width appeared to be narrower for 5A-SM compared to 5A-POPC, indicating decreased polydispersity of the rHDL particles. Both preparations appeared to be homogeneous in size as determined by DLS measurement. The volume averaged size distribution was 12.8±2.4 nm for POPC- and 9.6±0.7 nm for SM-rHDL. The binding of peptide to phospholipids was also confirmed by increased helicity of 5A in rHDL particles relative to free peptide by circular dichroism measurement (39 and 38% of apha-helix for POPC and SM-based rHDL, respectively, versus 19% for 5A peptide solution, Table 1).

TABLE 1
Physical characterization of rHDLs and free 5A peptide.

| | | | Relative Intensity by GPC (%) | | |
|---|---|---|---|---|---|
| Composition | Helicity (%) | Size (nm) | HDL | Liposome | Free peptide |
| 5A:POPC | 38.6 | 12.8 ± 2.4 | 85 | 3 | 12 |
| 5A:SM | 37.5 | 9.6 ± 0.7 | 90 | 0 | 10 |
| 5A | 18.9 | NA | 0 | 0 | 100 |

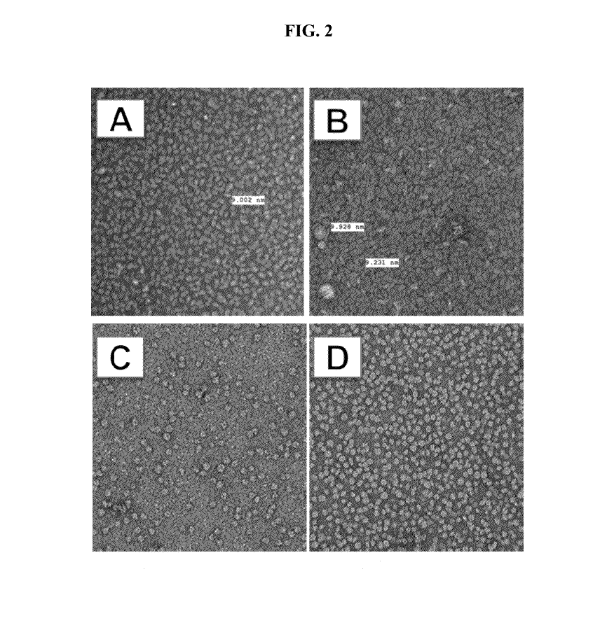

The morphology of rHDL particles was observed by transmission electron microscopy with negative staining (FIG. 2). Formation of highly homogeneous discoidal shaped rHDL of 8-12 nm diameter was observed for 5A-SM. The 5A-POPC rHDL had slightly larger size, greater heterogeneity, and a less defined shape. Some of these differences could be indicative of lower glass transition temperature of POPC relative to SM. The images of human HDL purified by gradient ultracentrifugation and a mixture of purified HDL and 5A-SM-HDL are shown in FIGS. 2 C and D, respectively. The images indicate that reconstituted HDL particles have similar size and morphology to the purified HDL. It is also visible that 5A-SM undergoes rapid remodeling in the presence of plasma purified HDL, as the shapes of HDL in the mixture appear to be more spherical (FIG. 2D) when compared to the reconstituted discoidal 5A-SM only (FIG. 2A).

3.2 The Effect of rHDL Composition on Cholesterol Efflux In Vitro

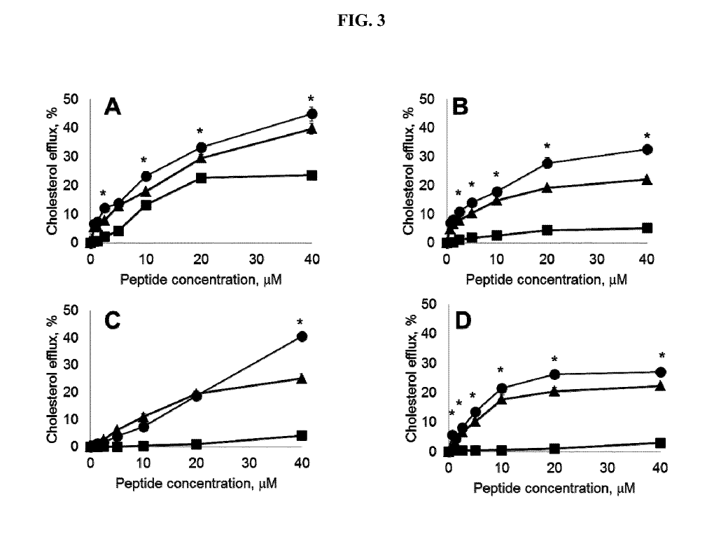

The effect of phospholipid composition of rHDL on in vitro cholesterol efflux via ABCA1 (FIG. 3A), ABCG1 (FIG. 3B) and SR-BI (FIG. 3C) was examined using BHK cells stably transfected with various human transporters that promote cholesterol efflux. The cells were loaded with radioactive cholesterol and percent cholesterol efflux was determined following incubation with different concentrations of 5A-POPC, 5A-SM and lipid free 5A peptide. The ABCA1 transporter is known to primarily promote the cholesterol efflux to lipid-poor or lipid-free apoA-I and thus was able to also promote cholesterol efflux to lipid-free 5A. Reconstituting 5A with either POPC or SM did not interfere with this process; in fact, both 5A-POPC and 5A-SM showed higher cholesterol efflux than lipid-free 5A and were similar to each other. In contrast, ABCG1 and SR-BI are known to primarily promote cholesterol efflux to HDL and not lipid-free apoA-I and thus showed relatively low cholesterol efflux to 5A. Reconstituting 5A with either PC or SM showed considerably greater efflux than lipid free 5A for both the ABCG1 and SR-BI transfected cell lines. At the highest concentration tested, both rHDL particles made with either SM or POPC were 40-50% more effective cholesterol acceptors from ABCG-1 and SR-BI transfected cells. Similar results, however, were also obtained from the mock transfected cells, which primarily efflux cholesterol by a passive diffusion process (30), suggesting that the increase in cholesterol efflux to rHDL was largely due to an increase by aqueous diffusion pathway. Although 5A-SM-HDL appeared to have slightly better in vitro efflux capacity than 5A-POPC-HDL for all 4 cell lines tested, overall the two rHDL preparations yielded similar results, regardless of the mechanism for cholesterol efflux from cells.

3.3 Dosing of rHDL Enhances Plasma Cholesterol Efflux Capacity

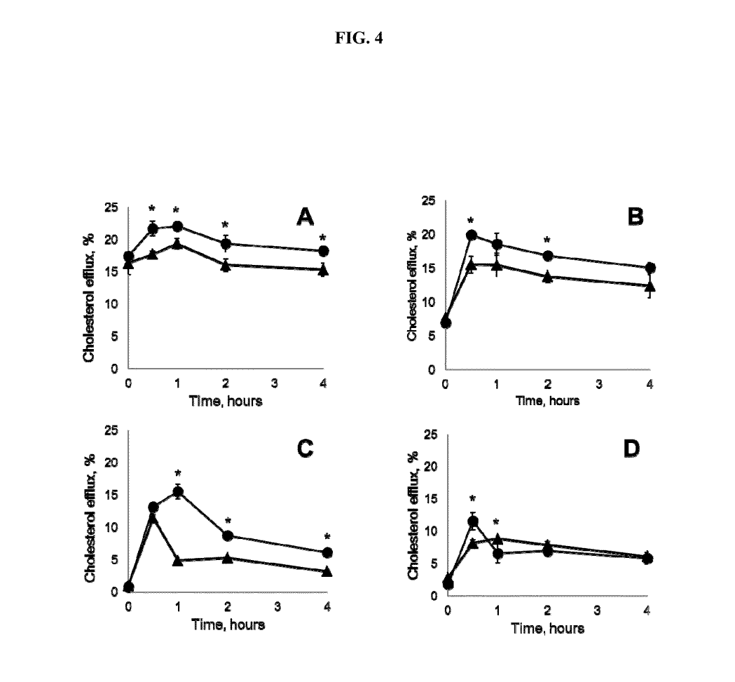

To examine how the plasma efflux capacity is altered following infusion of rHDL, 5A-SM and 5A-POPC were given by IV injection at 60 mg/kg to C57BL/6 mice. Plasma samples were collected at predetermined intervals and their capacity to efflux cholesterol from ABCA1, ABCG1 and SR-BI transfected BHK cell was determined. The percent cholesterol efflux was determined for plasma drawn at each time point and plotted as a function of time post infusion. Efflux by ABCA1 increased moderately and reached a maximum at 1 hour post infusion of 19% and 27% for 5A-POPC-rHDL and 5A-SM-rHDL, respectively. Overall, the duration and magnitude of the increase was greater for 5A-SM relative to 5A-POPC from the ABCA1 transfected cells (FIG. 4 A). An even larger increase compared to baseline was observed for SR-BI and ABCG1 plasma cholesterol efflux capacity was observed after infusion of 5A-SM and in both cases treatment with 5A-SM was superior to 5A-POPC in promoting cholesterol efflux (FIGS. 4B and C). ABCG1 efflux increased 2-fold at its peak for 5A-POPC, whereas 3-fold increase was observed for 5A-SM and it remained higher than 5A-POPC throughout the entire duration of study. Similarly, SR-BI efflux increased 1-fold for 5A-POPC over baseline at its peak, whereas 5A-SM increased 16-fold and its effect on increasing cholesterol efflux persisted longer. Cholesterol efflux from the mock transfected cells showed a much smaller increase after the infusion of 5A-SM and 5A-POPC, suggesting that the plasma changes induced by the infusion of 5A-SM and 5A-POPC result in greater efflux by ABCG1 and SR-BI.

### 3.4 HDL Remodeling in Plasma

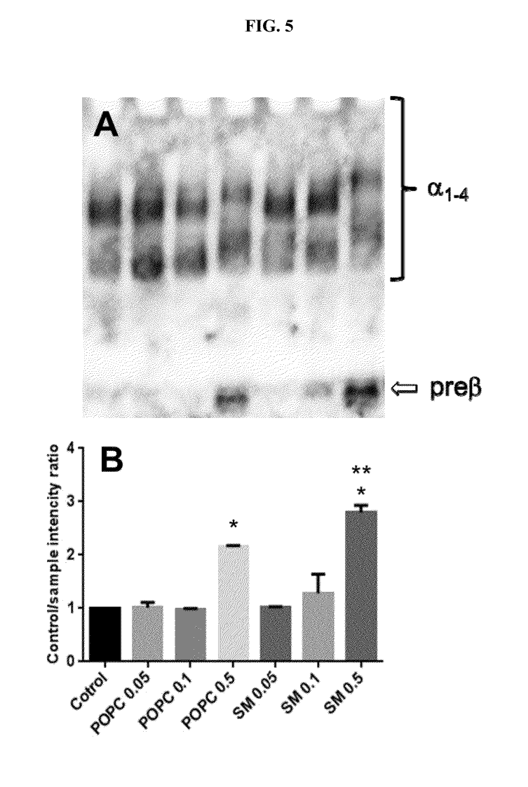

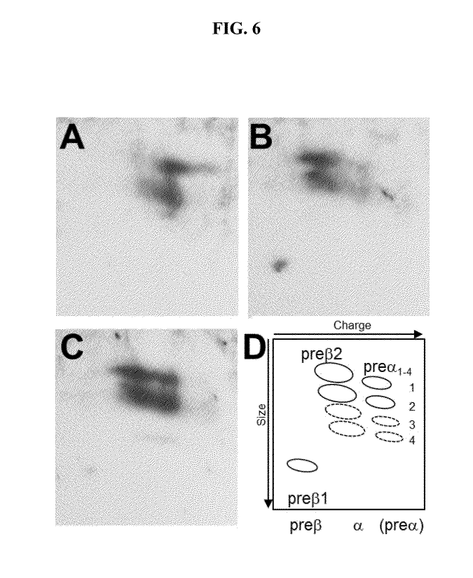

To assess how rHDL interacts with endogenous lipoprotein, 5A-POPC and 5A-SM were incubated with human plasma and HDL remodeling was visualized by 1-D and 2-D native page electrophoresis. Samples of 5A-SM and 5A-POPC were incubated for 30 minutes in human plasma at final peptide concentrations of 0.5, 0.1 and 0.05 mg/mL (FIGS. 5 and 6). The incubation resulted in measurable increase in the pre-β HDL band at 0.1 mg/mL concentration for 5A-SM, and both composition at 0.5 mg/mL concentration (accented with arrow in FIG. 5). The increase in pre-β HDL was statistically higher for 5A-SM relative to 5A-POPC at 0.5 mg/mL (p<0.5). Interestingly, some increase in α-HDL and decrease in pre-α-HDL was also observed for both 5A-POPC and 5A-SM relative to PBS control. The 2-D gel analysis of 5A-POPC and 5A-SM incubations at the highest concentration of 0.5 mg/mL confirmed the 1-D gel findings. An increase in pre-β HDL was observed for both 5A-POPC and 5A-SM; however, the effect is more prominent for 5A-SM than for 5A-POPC. Similar notable increases in α-1 and α-2 HDL intensities were observed for both rHDL preparations.

### 3.5 Anti-Inflammatory Properties

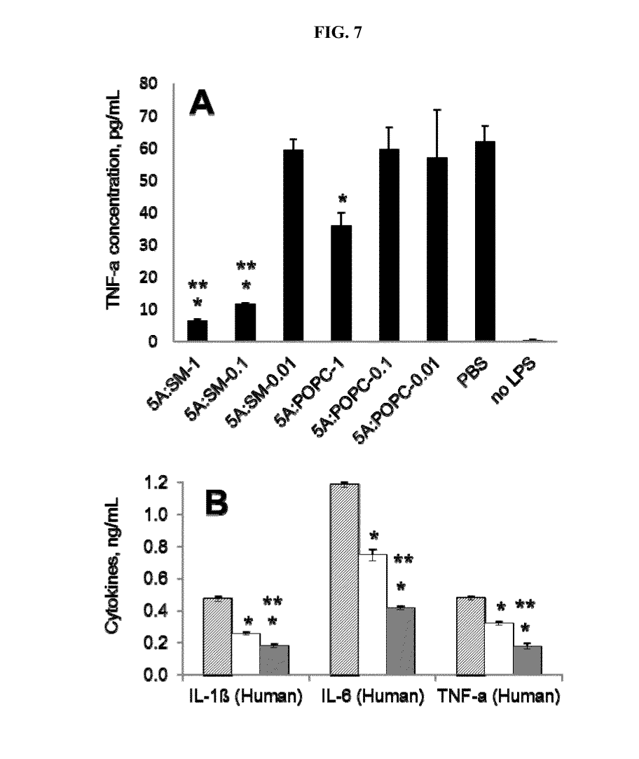

The effects of phospholipid composition on the anti-inflammatory properties of rHDL were examined in two separate experiments. First, mouse peritoneal macrophages were harvested, plated and pre-incubated with 5A-SM or 5A-POPC at 0.01, 0.1, and 1 mg/ml concentrations for 18 h. Following incubation, the media was replaced with fresh media without rHDL and cytokine release was stimulated by addition of LPS. Both lipid formulations of rHDL exhibited dose-dependent inhibition of cytokine release, although 5A-SM appeared to be significantly more potent than 5A-POPC (FIG. 7A). Both the high (1 mg/mL) and mid dose (0.1 mg/mL) of 5A-SM almost completely inhibited TNF-α release, while only the 1 mg/ml dose of 5A-POPC showed some protection. It is known that HDL can physically bind and neutralize LPS; however, this protection mechanism is unlikely to be applicable here, because the rHDL containing media was removed and a new media was added prior to LPS stimulation.

The ability of 5A-SM- and 5A-POPC-HDL to inhibit cytokine release in whole human blood stimulated by addition of PHA was also examined. The 5A-POPC and 5A-SM particles were added at 0, 0.01 and 0.1 mg/mL and pre-incubated for 1 h. Cytokine release was stimulated with PHA and the levels of IL-1β, IL-6 and TNF-α were determined (FIG. 7B). Similar to the mouse macrophages results, both rHDL exhibited dose-dependent inhibition of cytokine release relative to buffer control (p<0.005 for 5A-SM, and p<0.05 for 5A-POPC), and once again 5A-SM offered better protection relative to 5A-POPC (p<0.01).

3.6 Cholesterol Mobilization and Esterification in vivo

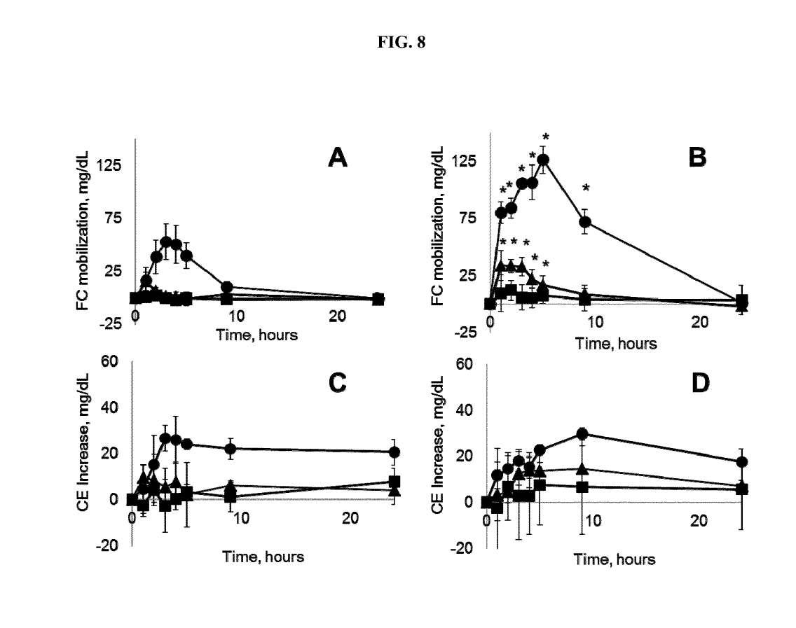

A rat dose-response study was performed in order to evaluate if apparent differences between 5A-SM- and 5A-POPC-HDL in cholesterol efflux in vitro translate to significant differences in cholesterol mobilization in vivo. Sprague Dawley rats received 10, 30 and 100 mg/kg doses of 5A-POPC- or 5A-SM-HDL by intravenous infusion. The dose was based on the peptide content of the dosing solutions. The increase in plasma free cholesterol (FC) (A, B) and cholesterol ester (CE) (C, D) are plotted in FIG. 8. The amount of mobilized free cholesterol was proportional to the injected dose and reached a maximum within 1-3 h post-infusion, depending on the dose. Most of the mobilized cholesterol appeared to be unesterified. Statistically significant differences in FC increase between 5A-POPC and 5A-SM was observed for 30 and 100 mg/kg dose at almost all time points (p<0.5 noted in FIG. 8). Approximately 2-3 times more cholesterol accumulated in the plasma compartment per dose of injected 5A-SM-HDL relative to 5A-POPC-HDL. The baseline level of plasma FC in rats is 30±5 mg/dL. Injection of 10 mg/kg of POPC-HDL showed limited cholesterol mobilization, as indicated by the insignificant increase of 3.1 mg/dL over baseline or an 11% increase at the highest FC level. By contrast, the same dose of 5A:SM-HDL increased FC to 12.9 mg/dl or a 39% increase from baseline (p<0.5). The difference between the two compositions was even more visible at higher doses. For 30 mg/kg infusions, 107% and 53% maximal increases in FC over baseline were observed 1-h post infusion for 5A-SM-versus 5A-POPC-HDL, respectively (p<0.05). The maximum FC mobilization was obtained 3 h post-dose for 100 mg/kg, and 105.6 mg/dL or a 387% increase from baseline levels was observed for 5A-SM-HDL and 53.3 mg/dL or 189% increase was detected for 5A-POPC-HDL (p<0.01 for two rHDL).

Cholesterol mobilization is the first important step in reverse cholesterol transport pathway; however, it is important to note that the lipid composition of the rHDL particles also affected cholesterol esterification by lecithin-cholesterol acyltransferase (LCAT), the second step in reverse cholesterol transport (11, 31). SM, unlike phosphatidylcholines, such as POPC, is not a substrate for LCAT and some reports indicate that SM inhibits LCAT activity (6). The increase in cholesterol ester (CE) was plotted at each time point following the infusion for each dose of 5A-SM- and 5A-POPC-HDL, as shown in FIGS. 8C and 8D. The dose dependent increase in CE formation was observed for both 5A-SM and 5A-POPC; however, the absolute amount of CE increase was lower than for FC, indicating that only a small fraction of mobilized FC converts to CE for both HDL compositions. These were no statistically significant differences in CE increase between 5A-POPC and 5A-SM. The CE increase was similar for 100 mg/kg dose of 5A-POPC and 5A-SM, as well as 30 mg/kg 5A-SM. These results indicate that rapid mobilization of large amount of FC likely saturates the endogenous LCAT esterification capacity.

### 4. Discussion

The most important finding of this study is the manner and extent to which alteration of rHDL phospholipid composition can alter the biological properties of rHDL, in regard to its cholesterol efflux ability and anti-inflammatory effect. It was found that 5A-SM effluxes more cholesterol by SR-BI and ABCG1 mechanisms relatively to 5A-POPC, and this ability is translated to greater cholesterol efflux capacity by plasma following infusion of rHDLs. ABCA1 efflux was not as much affected by lipid composition, as it is largely driven by lipid-free ApoA-I or the ApoA-I peptide component of HDL. Addition of 5A-POPC and 5A-SM to human plasma resulted in lipoprotein remodeling with notable formation of pre-β HDL particles and increase of α-HDL. The relative about of pre-3 HDL formation was higher for 5A-SM relative to 5A-POPC. The difference between cholesterol efflux ability by ABCG1 and SR-BI for the two rHDL lipid formulations was translated into notable difference in FC efflux in vivo. The 5A-SM-HDL mobilized roughly twice the amount of cholesterol relative to 5A-POPC-HDL at the same doses (FIG. 8). The absolute value of cholesterol ester increase was similar for both rHDL compositions at high doses indicating that availability of the endogenous LCAT might be a limiting factor for FC conversion to CE. The conversion of FC to CE following administration of SM-based rHDL was surprising, because SM is not a substrate for LCAT indicating that phosphatidylcholine species are likely to incorporate into SM-based rHDL during remodeling in vivo. Indeed, at the same level of cholesterol mobilization after dosing of either 100 mg/kg of 5A-POPC or 30 mg/kg 5A-SM, roughly same percent of FC was converted to CE. The effect of lipid composition on cholesterol esterification in vivo warrants more detailed study potentially using co-administration of recombinant LCAT or using isotope based assays for RCT. All mobilized cholesterol was successfully eliminated by 24 hours post rHDL infusion even after administration of 5A-SM at 100 mg/mL, as lipoprotein parameters returned to a baseline levels. This indicates that rHDL mobilized cholesterol is eventually likely eliminated by the liver either as cholesteryl ester or as unesterified cholesterol.

The anti-inflammatory properties of rHDL are also affected by lipid composition; 5A-SM was found to inhibit cytokine release more effectively than 5A-POPC. The greater anti-inflammatory activity of 5A-SM could be potentially related by greater cholesterol efflux by SR-BI mechanism. The SR-BI mediated efflux from the endothelial cells is known to cause activation of eNOS by kinase signaling (32), which could affect anti-inflammatory properties of rHDL. It is also possible that some of anti- and pro-inflammatory activity of rHDL is due to small amounts of signaling lipid impurities present in SM and POPC raw materials, such as sphingosine-1-phosphate (S1P), sphingosyl-phosphorylcholine (SPC) or lysophosphatidylcholine (LPC), or these could be generated after cellular uptake by rHDL. These molecules are known to be potent bioactive signaling molecules and could impact on the inflammatory cascade (32). Another potential mechanism of 5A-POPC and 5A-SM related anti-inflammatory activity could be mediated by ABCA1 efflux (33) and related to induction of ATF3 transcription modulator (34). The causes for relative differences between 5A-POPC and 5A-SM anti-inflammatory activities pertaining to these mechanisms have not yet been explored.

Similar differences between SM- and PC-based rHDL were reported in the several past studies. Proapolipoprotein A-I (proApoA-I) formulated with POPC showed approximately 10-fold lower free cholesterol mobilization in the HDL fraction relative to proApoA-I-SM-HDL, following the infusion into New Zealand White rabbits at 15 mg/kg doses (24). The comparison of in vitro cholesterol efflux by ApoA-I and ApoA-I-Milano formulated with either 1,2-dimyristoyl-rac-glycero-3-phosphocholine (DMPC) or SM was conducted in CHO cells, J774 macrophages and BHK cells transfected with ABCA1, non-functional ABCA1 mutant (W590S) and ABCG1 (9). SM-based rHDL exhibited higher cholesterol efflux than DMPC-based rHDL in all cell systems tested, and the difference was most significant in ABCG1 expressing cells. The phospholipid composition has less effect on ABCA1 mediated efflux, and ApoA-I-Milano-based rHDL was a slightly better cholesterol acceptor relative to ApoA-I-based rHDL. These findings are consistent with ABCA1 and ABCG1 efflux results in our study, using the 5A mimetic peptide.

The effects of phosphatidylcholine, fatty acid saturation, and addition of SM to rHDL on cholesterol efflux from J774 macrophages and esterified cholesterol uptake by HepG2 cells were also evaluated by Marmillot et al. (8). In general, the highest cholesterol efflux was observed for more saturated lipid based rHDL, such as fully saturated 1,2-Distearoyl-PC (DSPC, 18:0) and 1,2-Dioleoyl PC (DOPC, 18:1-mono-unsaturated). The incorporation of egg PC (unsaturated natural mixture), DLPC (18:2) and DLNPC (18:3) caused a significant decrease in efflux capacity. The addition SM increased cholesterol efflux. The CE uptake by HepG2 cells was also higher for rHDL containing more saturated lipids (50-100% of DSPC), while less uptake was observed for egg york PC (mostly unsaturated), DOPC, DLPC and DLNPC. Addition of SM at 40-80% of total lipid decreased the CE uptake by rHDL containing unsaturated lipids, but not for DSPC-based rHDL.

Another study of rHDL lipid composition on cholesterol efflux from mouse L-cell fibroblasts was performed by Davidson et al. but gave slightly different results (7). The SM-based rHDL exhibited slightly lower efflux capacity than unsaturated POPC and DOPC-based rHDL. The rHDL based on saturated PC, such as DSPC (18:0), DPPC (18:0), DMPC (14:0) and PSPC (16:0, 18:0), exhibited lower cholesterol efflux than HDL from POPC and DOPC. This efflux system examines physical uptake of free cholesterol from cell membranes similar to the mock efflux measurement in this study. The higher uptake by unsaturated lipids was attributed to the liquid crystalline state of unsaturated lipids at 37° C. However, the duration of rHDL incubation with cells, cell type, and rHDL concentration in the media were different in all the aforementioned studies, thus limiting direct comparison. It is important to note that commercially available sphingomyelins are all natural mixtures of lipids purified from different sources. Egg yolk SM, which is composed primary of palmitoyl acid (16:0, over 80%) was used in our study, while bovine brain SM (˜50% of 18:0 and ˜20% of 24:1) was used by Marmillot et al. and Davidson et al. (7, 8). Similar to PC, the fatty acid chain length and saturation is expected to affect the cholesterol binding of the SM bilayer and efflux capacity of SM-based rHDL.

An important issue is if the greater cholesterol mobilization efficiency by SM-based rHDL relative to POPC-based rHDL will result in anti-atherosclerotic efficacy at lower doses. Some of our previous data is suggestive of such a response (26). In two independent studies 4-month-old apoE-KO mice on a normal chow diet were injected with 30 mg/kg of: i) 5A:POPC-HDL or POPC liposome control; and ii) 5A-SM-DPPC-HDL or saline control for 13 weeks (3 times a week) and then analyzed for percentage of surface area covered by aortic plaque. The atheroma area was smaller following 5A-SM-DPPC-HDL treatment (˜3.5%) relative to 5A-POPC-HDL treatment (˜6%). Injections of both rHDL showed statistically significant improvement relative to placebo or POPC liposome controls (26). However, an evaluation of 5A-SM and 5A-POPC compositions in a single anti-atherosclerosis study in ApoE knockout mice has not yet been performed. The results of the current study provide important information about dose response, duration of the effect, and will guide us in selection of doses and administration frequencies for a future anti-atherosclerosis study. In addition, how the phospholipid composition of rHDL affects biodistribution and pharmacokinetic properties of rHDL, i.e., circulation half-life, clearance, volume of distribution, remains to be explored.

Another critical question is the importance of effective interaction of rHDL with LCAT for successful reduction of atheroma. While SM is not a substrate of LCAT, fully saturated PC, such as DPPC, is capable of serving as a substrate for LCAT and, like SM, is similar in its strong cholesterol binding properties. The unsaturated PC, such as POPC and DLPC, are reported to be more efficient substrates for LCAT but unsaturation results in weaker cholesterol efflux. It may be preferable to investigate the effect of lipid composition cholesterol esterification in vivo using either full-length ApoA-I or ApoA-I mimetic peptides optimized specifically for LCAT activation. The results of this study are significant because they shift the focus of rHDL research from protein or peptide component to an equally important phospholipid component. While several rHDL products have already been translated to the clinic, the doses required for clinical efficacy are relatively large (40 mg/kg and 45 mg/kg), requiring as much as 5 grams of Apolipoprotein per infusion (4, 5, 15, 16). Administration of such high doses has been associated with toxicity, which may have resulted from various impurities, including: cholate used in preparation sHDL particles, oxidized lipids, potentially immunogenic ApoA-I aggregates, bacterial proteins, DNA and endotoxins from recombinant processing during manufacturing of ApoA-I. A high dose of 80 mg/kg of CSL-111 also had to be discontinued in a Phase 2 study due to toxicity (5). High doses also require the manufacture of large quantities of very pure protein or ApoA-I mimetic peptide and rHDL nanoparticles, which is technically difficult and costly. Hence, the ability to attenuate pharmacological efficacy of rHDL by optimization of its phospholipid composition could potentially lead to the creation of new rHDL therapeutics that are effective at a fraction of the currently used clinical doses.

The end goal of rHDL infusion therapy is to rapidly remove excess cholesterol from arterial plaque and reduce the risk of future heart attack and, therefore, the ability of rHDL to mimic all functions of natural HDL i.e., endothelial cell layer penetration, cholesterol efflux from foam cells, anti-inflammatory and anti-oxidant functions, interaction with LCAT for conversion of FC to CE, and transport of mobilized and esterified cholesterol to the liver for elimination is highly important. As this study and previous published literature indicate, all of these functions of rHDL are potentially affected by its phospholipid composition. Therefore, understanding the mechanism of how lipid composition alters efficacy and safety of rHDL is critical for successful clinical translation of this novel class of cardiovascular drugs.

### Example 2

### The Effect of Phospholipid Composition of Reconstituted HDL on its Cholesterol Efflux and Anti-Inflammatory Properties

### Abstract

The goal of this study was to understand how the reconstituted HDL (rHDL) phospholipid composition affects its cholesterol efflux and anti-inflammatory properties. 5A, an apolipoprotein A-I mimetic peptide (SEQ ID NO:1), was combined with either sphingomyelin (SM) or palmitoyl-2-oleoyl-sn-glycero-3-phosphocholine (POPC). Both lipid formulations exhibited similar in vitro cholesterol efflux by ABCA1, but 5A-SM exhibited higher ABCG1 and SR-BI mediated efflux relative to 5A-POPC (p<0.05). Injection of both rHDLs in rats resulted in mobilization of plasma cholesterol, although the relative potency was 3-fold higher for the same doses of 5A-SM than for 5A-POPC. Formation of pre-β HDL was observed following incubation of rHDLs with both human and rat plasma in vitro, with 5A-SM inducing higher extent of pre-β formation relative to 5A-POPC. Both rHDLs exhibited anti-inflammatory properties, but 5A-SM showed higher inhibition of TNF-α, IL-6 and IL-1β release than did 5A-POPC (p<0.05). Both 5A-SM and 5A-POPC showed reduction in total plaque area in ApoE−/− mice, but only 5A-SM showed a statistically significant reduction over placebo control and baseline (p<0.01). The type of phospholipid used to reconstitute peptide has significant influence on rHDL anti-inflammatory and anti-atherosclerosis properties.

### 1. Introduction

Infusions of cholesterol-free reconstituted high-density lipoprotein (rHDL) particles have been shown to rapidly reverse atherosclerosis in a wide variety of animal models and in clinical trials of acute coronary syndrome (ACS) patients (Remaley, A. T., Amar, A., Sviridov, D. (2009) Expert Rev Cardiovasc Ther, 6(9): 1203-15; Newton, R. S., Krause, B. R. (2002) Atheroscler Suppl, 3(4): 31-8; Badimon, J. J., Badimon, L., Fuster, V. (1990) J Clin Invest, 85(4): 1234-41; Nissen, S. E., et al. (2003) JAMA, 290: 2292-2300; and Tardif, J. C., et al. (2007) JAMA; 297: 1675-1682). While a significant emphasis has been placed on investigating the HDL protein component i.e., Apolipoprotein A-I (ApoA-I), ApoA-I mutants and mimetic peptides, the importance of HDL phospholipid composition not been systematically investigated. Lipid represents 50 to 80% of the total HDL mass and is known to affect particle stability in vivo, cholesterol efflux from macrophages, ability to interact with lecithin-cholesterol acyltransferase (LCAT) and cholesterol elimination (Rye, K. A., Hime, N. J., Barter, P. J. (1996) J. Biol Chem., 271: 4243-4250; Davidson, W. S., et al. (1995) J. Biol. Chem., 270: 5882-5890; Marmillot, P., Patel, S., Lakshman, M. R. (2007) Metabolism, 56(2): 251-9; Ma, C. I., et al. (2012) Biochem Cell Biol, 90(5): 636-45; Sparks, D. L., et al. (1995) J. Biol. Chem., 270: 26910-26917; and Parks, J. S., et al. (2000) J Lipid Res, 41: 546-553). Lipid composition also largely defines the size, net charge and rigidity of the rHDL particles—all are important factors in the pharmacokinetic and pharmacodynamics properties of rHDL. The investigation of the effects of lipid composition on the resulting rHDL properties in vitro and in vivo is the focus of this article. By better defining the lipid effect of rHDL, the goal was to be able to favorably alter the potency and safety of rHDL, and ultimately advance clinical translation of these potentially life-changing nanomedicines.

The phospholipid composition of endogenous HDL contains phosphatidylcholines (PC), sphingomyelin (SM) and small amounts of lysophosphatidylcholine (LPC), phosphatidylethanolamine (PE), phosphatidylinositol (PI) and other lipids (Kontush, A., Lhomme, M., Chapman, M. J. (2013) J Lipid Res, 54: 2950-2962; Camont, L., et al. (2013) Arterioscler Thromb Vasc Biol, 33(12): 2715-2723; and Kontush A., et al. (2007) Arterioscler Thromb Vasc Biol., 27: 1843-1849). The differences in lipid composition of various subclasses of HDL and how they relate to disease have been gaining attention in recent years (Kontush, A., Lhomme, M., Chapman, M. J. (2013) J Lipid Res, 54: 2950-2962; Camont, L., et al. (2013) Arterioscler Thromb Vasc Biol, 33(12): 2715-2723; and Kontush A., et al. (2007) Arterioscler Thromb Vasc Biol., 27: 1843-1849). However, a single source of lipid is used for preparation of reconstituted HDL for most academic research and clinical development (Remaley, A. T., Amar, A., Sviridov, D. (2009) Expert Rev Cardiovasc Ther, 6(9): 1203-15; Newton, R. S., Krause, B. R. (2002) Atheroscler Suppl, 3(4): 31-8; Badimon, J. J., Badimon, L., Fuster, V. (1990) J Clin Invest, 85(4): 1234-41; Nissen, S. E., et al. (2003) JAMA, 290: 2292-2300; Tardif, J. C., et al. (2007) JAMA; 297: 1675-1682; Carlson, L. (1995) Nutr Metab Cardiovasc Dis, 5: 85-91; Nanjee, M. N., et al. (1999) Arterioscler Thromb Vasc Biol, 19(4): 979-89; Marchesi, M., et al. (2004) J Pharmacol Exp Ther, 311(3): 1023-31; and Hippalgaonkar, K., et al. (2010) AAPS PharmSciTech, 11(4): 1526-1540). The first lipid used to prepare rHDLs for human testing was soybean phosphatidylcholine (soyPC), because this lipid was shown to be safe for parenteral nutrition products and is readily commercially available in infusion grade (Hippalgaonkar, K., et al. (2010) AAPS PharmSciTech, 11(4): 1526-1540). Thus recombinant proapolipoprotein A-I (a product of UCB Pharma, Anderlecht, Belgium) and blood purified ApoA-I (CSL-111, a product of CSL Behring, Australia) were reconstituted with soyPC (Carlson, L. (1995) Nutr Metab Cardiovasc Dis, 5: 85-91; and Nanjee, M. N., et al. (1999) Arterioscler Thromb Vasc Biol, 19(4): 979-89; Marchesi, M., et al. (2004) J Pharmacol Exp Ther, 311(3): 1023-31). As a greater variety of infusion grade chemically defined lipids became commercially available, the phospholipid composition used in the preparation of rHDL diversified. A 1-palmitoyl-2-oleoyl-sn-glycero-3-phosphocholine (POPC) was first used in ETC-216 (rApoA-I-Milano), and then a mixture of 1,2-dipalmitoyl-sn-glycero-3-phosphocholine (DPPC) and SM was used for ETC-642, a peptide-based rHDL (Marchesi, M., et al. (2004) J Pharmacol Exp Ther, 311(3): 1023-31; and Di Bartoloa, et al. (2011) Atherosclerosis, 217: 395-400). Most recently, SM was used in CER-001, another newly developed rApoA-I-based rHDL (International Application No. WO/2012/109162). Both sphingomyelin and saturated fatty acid containing lipids like DPPC have superior cholesterol binding capacity relative to unsaturated POPC and soyPC (Ohvo-Rekilai, H., Ramstedt et al. (2002) Prog Lipid Res, 41(1): 66-97; and Ramstedt, B., Slotte, J. P. (1999) Biophys. J. 76: 908-915). In fact, SM has the strongest cholesterol binding affinity governed by Van der Waals' interaction due to the mismatched fatty acid lengths (Ohvo-Rekilä, H., Ramstedt et al. (2002) Prog Lipid Res, 41(1): 66-97). SM is also known to form lipid-raft sections in cellular membrane that contain up to 50% of cholesterol (Silva. L. C., Futerman, A. H., Prieto, M. (2009) Biophys. J. 96(8): 3210-22). The rHDLs made from saturated lipids exhibited greater cholesterol efflux from macrophages in vitro (Marmillot, P., et al. (2007) Metabolism, 56(2): 251-9; Ma, C. I., et al. (2012) Biochem Cell Biol, 90(5): 636-45; and Sparks, D. L., et al. (1995) J. Biol. Chem., 270: 26910-26917) and cholesterol mobilization in vivo (International Patent Publication No. WO 2003/096983). However, unsaturation enhances rHDL interaction with LCAT (Parks, J. S., et al. (2000) J Lipid Res, 41: 546-553), whereas SM was reported to inhibit cholesterol esterification in vitro (Rye, K. A., et al. (1996) J. Biol Chem, 271: 4243-4250).

In this example, it was hypothesized that higher cholesterol binding affinity of SM relative to POPC would translate into greater cholesterol efflux both in vitro and in vivo. The ApoA-I mimetic, 5A, a 37-amino acid-long bi-helical peptide (Sethi, A. A., et al. (2008) J Biol Chem, 283:32273-32282), was used to form SM and POPC-based HDL particles. The 5A based HDL has several similar features to HDL prepared from the full-length ApoA-I, namely, an equivalent ability to promote cholesterol efflux in vitro and in vivo (Sethi, A. A., et al. (2008) J Biol Chem, 283:32273-32282; and Amar, M. J. A., et al. (2010) J Pharmacol Exp Ther, 334: 634-641), and similar abilities in reducing the development of atherosclerosis (Amar, M. J. A., et al. (2010) J Pharmacol Exp Ther, 334: 634-641) and suppressing inflammation (Tabet, F., et al. (2010) Arterioscler Thromb Vasc Biol, 30: 246-252). In this study, differences in cholesterol efflux and inflammatory cytokine release inhibition were examined for 5A-POPC and 5A-SM. In addition, the ability of the two different lipid preparations of 5A were tested in vivo on their ability mobilize and esterify cholesterol and on their effect in atherosclerosis development.

### 2. Materials and Methods

### 2.1 Materials

5A (DWLKAFYDKVAEKLKEAFPDWAKAAYDKAAEKAKEAA; SEQ ID NO:1) was synthesized by Genscript (Piscataway, N.J.), using solid-phase Fmoc (9-fluorenylmethyl carbamate) protection chemistry and was purified with reverse phase chromatography. Peptide purity was >95% as determined by HPLC. Egg sphingomyelin and 1-palmitoyl-2-oleoyl-sn-glycero-3-phosphocholine (POPC) were purchased from Avanti Polar Lipids (Alabaster, Ala.) and Nippon Oil and Fat (Osaka, Japan). All other materials were obtained from commercial sources.

### 2.2 Preparation and Characterization of 5A Phospholipid Complexes

High-density lipoprotein-like 5A-POPC and 5A-SM complexes were prepared by a co-lyophilization procedure (25, 26). Peptide and phospholipids were dissolved in glacial acetic acid and mixed at 1:1.25 w/w ratio (approximately 1:7 molar ratio) and lyophilized. The powder was hydrated with bicarbonate buffered saline and cycled between 50° C. and room temperature to facilitate 5A-lipid binding. The resulting HDL complexes were analyzed by gel permeation chromatography, with UV detection at 220 nm, using Tosoh TSK gel G3000SWx1 column (Tosoh Bioscience, King of Prussia, Pa.). The HDL hydrodynamic diameters were determined by dynamic light scattering (DLS), using a Zetasizer Nano ZSP, Malvern Instruments (Westborough, Mass.). The volume intensity average values were reported. The alpha-helical contents of free and lipid bound 5A peptide were determined by Jasco J715 (Jasco, Easton, Md.) circular dichroism spectropolarimeter. Samples at 0.1 mg/mL concentration were loaded into a quartz cuvette (d=0.2-cm path length), and CD spectra from 185 to 240 nm were recorded at 24° C. Data were normalized by calculating the mean residue ellipticity (θ).

Transmission electron microscopy (TEM) images were obtained using JEM 1200EX electron microscope (JEOL USA) equipped with an AMT XR-60 digital camera (Advanced Microscopy Techniques Corp). Images were acquired at magnifications from 60,000 to 150,000-fold. HDL samples, 3 μL aliquots, were deposited on carbon film-coated 400 mesh copper grids (Electron Microscopy Sciences) and dried for 1 min. The samples were negatively stained with 1% uranyl acetate solution; the grids were blotted with tissue and dried before TEM observation.

2.3 Cholesterol Efflux Assay in vitro

Cholesterol efflux studies were performed, as described previously (25, 26). Briefly, BHK-mock and BHK stably transfected cells with human ABCA1 cDNA, ABCG1 cDNA and SR-BI cDNA were labeled for 24 h with 1 μCi/ml of 3H-cholesterol in minimum essential medium plus 10% fetal calf serum. Transporters were induced with 10 nM mifepristone in Dulbecco's modified Eagle's medium (DMEM), containing 0.2 mg/ml of fatty acid-free bovine serum albumin for 18 h. Following induction of the transporters, the cells were washed and rHDLs or 5A were added at 0.1, 2.5, 5, 10, 20 and 40 mM peptide concentrations in DMEM-BSA-mifepristone media. After 18 h of incubation, media were collected and filtered using 24-well, 25-μm pore size plate filter (Whatman—GE Healthcare, Pittsburgh, Pa.) and cells were lysed in 0.4 ml of 0.1% SDS and 0.1 N NaOH. Radioactive counts in media and cell fractions were measured by liquid scintillation counting, and percent cholesterol effluxed was calculated by dividing media count by the sum of media and cell counts.

### 2.4 Plasma HDL Remodeling

Remodeling of rHDL in plasma was assessed by addition of 50 μL of 5, 1 or 0.5 mg/ml of 5A-SM or 5A-POPC to 450 μL of pooled human plasma. The final concentrations of peptide in plasma were 0.5, 0.1 and 0.05 mg/mL respectively. Plasma incubation with the same volume of PBS was used as a control. Samples were incubated at 37° C. for 1 hour under shaking at 300 rpm prior to electrophoresis. The various sub-classes of HDL were separated by size and charge by one- or two-dimension native page gel electrophoresis and visualized by western blot using anti-ApoA-I antibody.

To visualize native HDL particles separated by size, samples were subjected to electrophoresis using 10-well Tris-Borate-EDTA gradient (3-25%) acrylamide mingels (Jule, Inc., Milford, Conn.) (28). For each well, 5 pl of rat and human plasma incubated with or without rHDL was mixed with 5 pl of 2×TBE sample buffer and 6 pl of the resulting mixtures were loaded per well. Gels were run at 200 V until the sample dye was 2.5 cm away from the bottom of the gel. Proteins were visualized by western blot by transfer onto PVDF membrane and incubation overnight with anti-human and anti-rat apoA-I-HRP conjugated antibody (Meridian Life Science, Memphis, Tenn.). Antibody solution was prepared by mixing 3 pl of antibody with 40 mL of antibody dilution buffer. Images were acquired on an Alpha Innotech Chemi Imager 5500 and the Alpha Ease FC program was used for spot densitometry.

To visualize native HDL particles separated by charge and size, samples were subjected to native-native 2D gel electrophoresis (Freeman, L. A. (2013) Western blots. Methods Mol Biol., 1027:369-85). For each sample, 2 pl plasma incubated with rHDLs or PBS control, 4 pl sample buffer and 8 pl Tris tricine buffer were combined. A 10.0 pl aliquot was added per well of the first-dimension gel (0.7% agarose), followed by 2nd-dimension electrophoresis on a 3-25% acrylamide gradient TBE mini-gels (Jule). The western blot analysis and visualization was performed as described above. The classification of various HDL sub-classes was performed in accordance with Asztalos et al (30).

### 2.5 Inhibition of Cytokine Release Testing

Inhibition of cytokine release from murine peritoneal macrophages following lipopolysaccharides (LPS) stimulation was determined for 5A-SM, 5A-POPC, free 5A peptide and PBS controls. Mouse peritoneal lavage was performed with 5 mL of sterile PBS. Peritoneal macrophages were washed, collected by centrifugation and reconstituted in DMEM containing 10% fetal bovine serum (FBS). Cells were plated, attached, washed twice with PBS, stripped by trypsin and counted. Macrophages were seeded at 100,000 cells per well, incubated for 2 h, following by addition of rHDLs at 0.01, 0.1 and 1 mg/mL peptide concentrations for 18 h. The cells were washed again, treated with 1 pg/mL of LPS for 2 h, washed again and incubated for 6 h. The media was collected and the levels of TNF-α were determined by for BioLegend ELISA kits (San Diego, Calif.) as per the manufacturer's instructions.

Determination of cytokine release inhibition in whole human blood was performed by pretreating heparinized human blood for 1 h with 5A-SM, 5A-POPC, free 5A peptide controls at 0, 0.01, and 0.1 mg/mL peptide concentrations. Cytokine release was stimulated by addition of 1 μg/mL of phytohemaglutinin-M (PHA) followed by an overnight incubation. The cells were collected by centrifugation and the levels of IL-β, IL-6 and TNF-α were measured by Human Prolnflammatory-4 I tissue culture kit using SECTOR® Imager 2400 from Meso Scale Discovery (Rockville, Md. Rockville, Md.).

2.6 Cholesterol Mobilization and Esterification in vivo.

Sprague-Dawley rats (8 weeks old) were purchased from Charles River Breeding Laboratories (Portage, Mich.) and were fed a regular rodent chow diet. To determine the dose response, 5A-SM and 5A-POPC were administered intravenously via tail veil at 30 and 100 mg/kg doses based on the peptide amount. The plasma samples were collected pre-dose and at 0.5, 1, 2, 4, 6, 8, 12, and 24 h after the infusion and the levels of plasma phospholipids (PL), total cholesterol (TC) and unesterified or free cholesterol (FC) were determined by enzymatic analysis using commercially available kits (Wako Chemicals, Richmond, Va.). Cholesterol ester level was calculated as a difference between TC and FC levels at each time point.

### 2.7 Distribution of Mobilized Cholesterol in Lipoproteins

The rat sera samples were analyzed to assess cholesterol distribution between VLDL, LDL and HDL lipoprotein fractions. Separation of lipoproteins was performed on Waters HPLC system equipped with Superose 6, 10/300 GL column (GE Healthcare, Piscataway, N.J.) and a fraction collector. Rat sera prior to dosing and 30 min post-injection of 100 mg/mL 5A-SM and 5A-POPC were analyzed. A 50 μl aliquots were injected and eluted with saline solution at 1 ml/min. Elution fractions (0.5 mL) were collected with a Waters fraction collector and analyzed for total cholesterol by an enzymatic kit. The levels of cholesterol were plotted as a function of fraction time.

### 2.8 Anti-Atherosclerotic Activity in ApoE−/− Mice.

Eight-week-old male apoE knockout (apoE−/−) mice were fed a high-fat and -cholesterol diet (HFD, 21% fat, 34% sucrose, and 0.2% cholesterol, Harlan, T. D. 88137) for 14 weeks to develop atherosclerotic lesions, at which point mice were either sacrificed (baseline) or switched to chow diet for 6 weeks. Coincident with the switch to chow diet, mice were randomized into 3 groups and received intraperitoneal injection three times a week (Monday, Wednesday, and Friday) of either 5A-POPC or 5A-SM at 50 mg/kg dose or an equivalent volume (200 μL) of PBS control for 6 weeks. The left ventricle of the heart was perfused with PBS, followed by a fixative solution (4% paraformaldehyde in PBS). The aorta was dissected from its origin in the heart to the ileal bifurcation and stained with oil red O solution, then destained for 30 min in 70% ethanol, and finally washed in water. After removal of any remaining adventitial fat, aortas were cut longitudinally and pinned flat onto a black-wax plate. The percentage of the plaque area stained by oil red O with respect to the total luminal surface area was quantified with Image J analysis software. To quantify the extent of the atherosclerotic lesions in the aortic root, the atherosclerotic lesions in the aortic sinus region was examined at 3 locations, each separated by 80 μm. The largest plaque of the three valve leaflets was adopted for morphological analysis. The lipid-burden plaque areas at the aortic sinus were determined by oil red O staining.

### 3. Results

3.1 Preparation and Characterization of rHDLs

Reconstituted HDL (rHDL) particles were prepared by combining 5A peptide with either SM or POPC, using a co-lyophilization procedure. Preliminary studies were performed to determine the optimal weight ratio of peptide to phospholipids for formation of homogeneous rHDL complexes. The ratio of 1:1.25 wt/wt or 1:7 mole:mole of peptide to phospholipid was selected. Higher ratios of lipid to peptide resulted in greater rHDL heterogeneity and the presence of liposome impurities. Lower amounts of lipid resulted in the presence of some lipid free peptide. The optimum weight ratio of peptide to phospholipid ranged between 1:1.25 and 1:1.5 and resulted in homogenous rHDL particle size distribution. The 1:1.25 ratio was chosen as the preferred formulation.

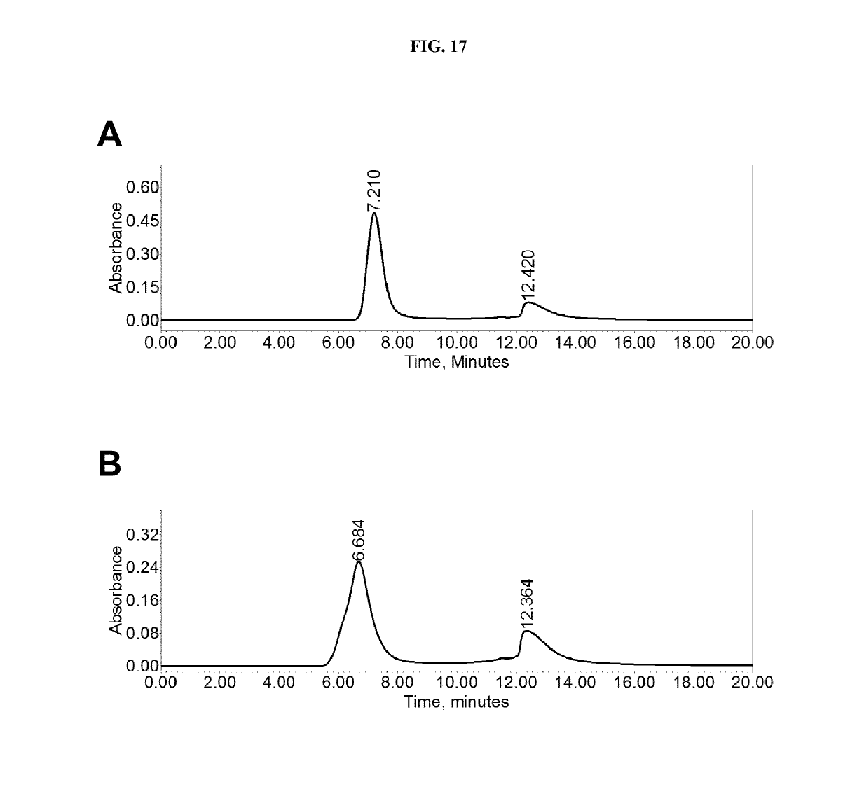

The analysis of the purity of rHDL complexes was performed by gel permeation chromatography (GPC) shown in FIG. 17. The results indicate formation of rHDL (elutes at 7 min) and a presence free 5A peptide (12 min retention time). The POPC-HDL contained slightly higher percent of unbound 5A relative to the SM-HDL preparation. The HDL peak width appeared to be narrower for 5A-SM compared to 5A-POPC, indicating decreased polydispersity of the SM-rHDL particles. Both preparations appeared to be homogeneous in size as determined by DLS measurement. The volume averaged size distribution and width of distribution were 12.8 nm and 2.4 nm for POPC-rHDL and 9.6 and 0.7 nm for SM-rHDL. The binding of peptide to phospholipids was also confirmed by increased helicity of 5A in rHDL particles relative to free peptide by circular dichroism measurement. The alpha-helix content lipid-bound 5A was 37.9±0.7% and 37.6±0.4% for POPC and SM rHDL, respectively, versus 18.8±0.3% for the free 5A peptide solution (n=3, mean±SD).

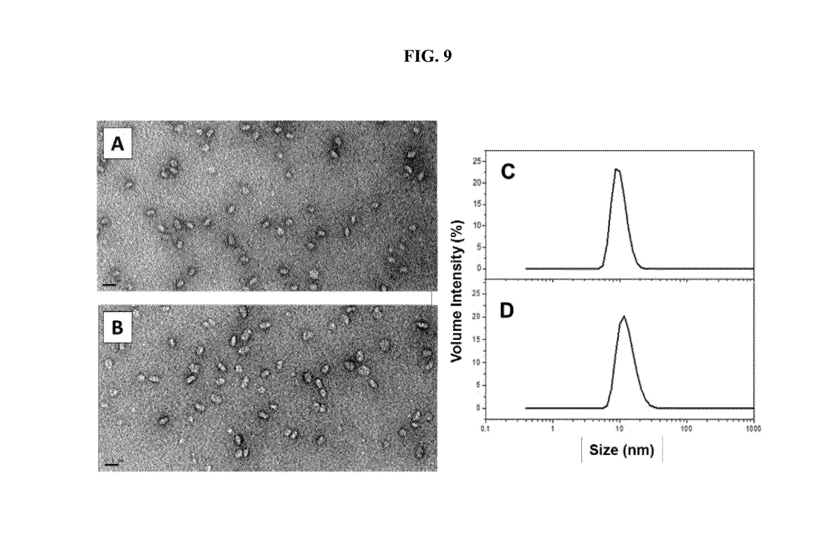

The morphology of rHDL particles was observed by transmission electron microscopy with negative staining (FIG. 9). Formation of highly homogeneous discoidal shaped rHDL of 8-12 nm diameter was observed for 5A-SM. The 5A-POPC rHDL had slightly larger size, greater heterogeneity, and a less defined shape. Some of these differences could be indicative of lower glass transition temperature of POPC relative to SM.

3.2 The Effect of rHDL Composition on Cholesterol Efflux in vitro

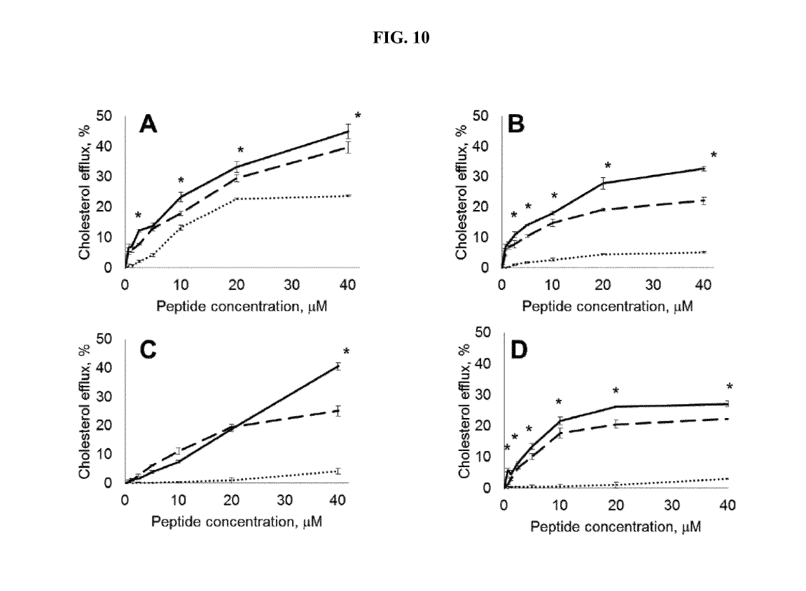

The effect of phospholipid composition of rHDL on in vitro cholesterol efflux via ABCA1 (FIG. 10A), ABCG1 (FIG. 10B) and SR-BI (FIG. 10C) was examined, using BHK cells stably transfected with various human transporters that promote cholesterol efflux. The cells were loaded with radioactive cholesterol and percent cholesterol efflux was determined following incubation with different concentrations of 5A-POPC, 5A-SM and lipid free 5A peptide. The ABCA1 transporter is known to primarily promote the cholesterol efflux to lipid-poor or lipid-free apoA-I and thus was able to also promote cholesterol efflux to lipid-free 5A. Reconstituting 5A with either POPC or SM did not interfere with this process; in fact, both 5A-POPC and 5A-SM showed higher cholesterol efflux than lipid-free 5A and were similar to each other. In contrast, ABCG1 and SR-BI are known to primarily promote cholesterol efflux to HDL and not lipid-free apoA-I and thus showed relatively low cholesterol efflux to 5A. Reconstituting 5A with either PC or SM showed considerably greater efflux than lipid free 5A for both the ABCG1 and SR-BI transfected cell lines. At the highest concentration tested, both rHDL particles made with either SM or POPC were 40-50% more effective cholesterol acceptors from ABCG-1 and SR-BI transfected cells. Similar results, however, were also obtained from the mock transfected cells, which primarily efflux cholesterol by a passive diffusion process (31), suggesting that the increase in cholesterol efflux to rHDL was largely due to an increase by aqueous diffusion pathway. Although 5A-SM-HDL appeared to have slightly better in vitro efflux capacity than 5A-POPC-HDL for all 4 cell lines tested, overall the two rHDL preparations yielded similar results, regardless of the mechanism for cholesterol efflux from cells.

### 3.3 HDL Remodeling in Plasma

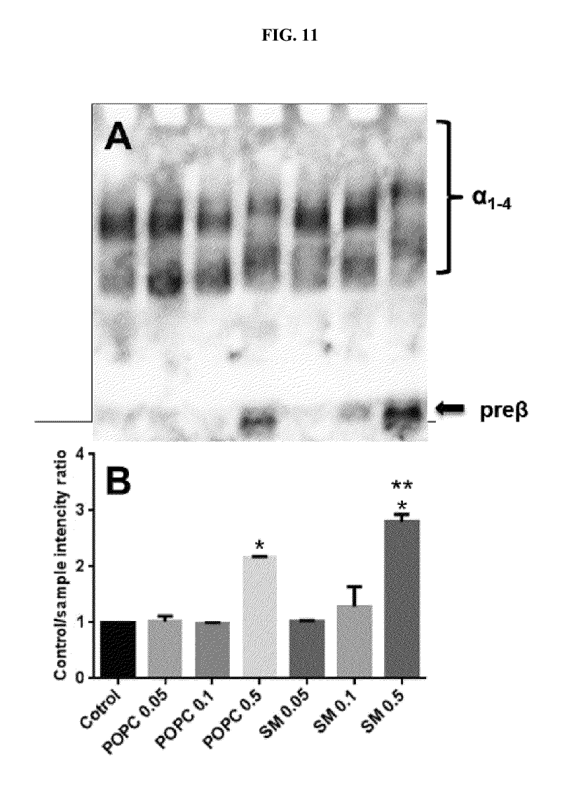

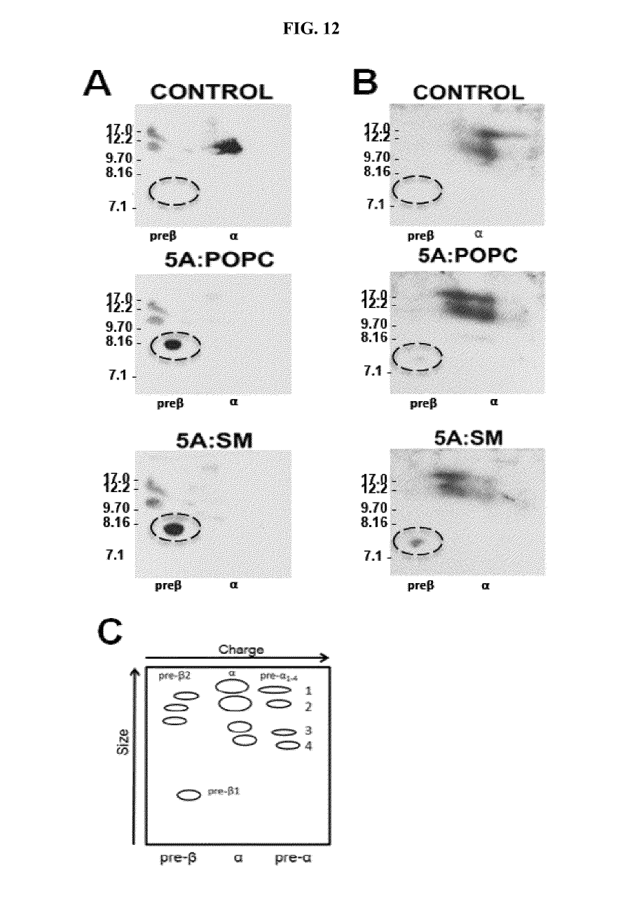

To assess how rHDL interacts with endogenous lipoprotein, 5A-POPC and 5A-SM were incubated with human plasma and HDL remodeling was visualized by 1-D and 2-D native page electrophoresis. Samples of 5A-SM and 5A-POPC were incubated for 30 minutes in human plasma at final peptide concentrations of 0.5, 0.1 and 0.05 mg/mL. The HDL sub-fractions were separated by size by 1-D native page electrophoresis (FIG. 11). The incubation resulted in measurable increase in the pre-β HDL band at 0.1 mg/mL concentration for 5A-SM and for both rHDL composition at 0.5 mg/mL concentration. The increase in pre-β HDL was statistically higher for 5A-SM relative to 5A-POPC at 0.5 mg/mL (p<0.05). The 2-D gel analysis of 5A-POPC and 5A-SM incubations at the highest concentration of 0.5 mg/mL confirmed the 1-D gel findings (FIG. 12B). An increase in pre-β HDL (highlighted by dashed circles in FIG. 12) was observed for both 5A-POPC and 5A-SM; however, the effect is more prominent for 5A-SM than for 5A-POPC. Similar notable increases in α-1 and α-2 HDL intensities were observed for both rHDL preparations. The incubation of 5A-SM and 5A-POPC with rat plasma also showed complete disappearance of α-HDL spots and appearance of prominent pre-β HDL. These findings suggest that both 5A-POPC and 5A-SM can readily interact with native HDL to induce remodeling in plasma. The HDL remodeling was somewhat more prominent upon 5A-SM incubation compared with 5A-POPC, which correlates well with higher anti-atherosclerotic activity in vivo of 5A-SM shown later in the example.

### 3.5 Anti-Inflammatory Properties

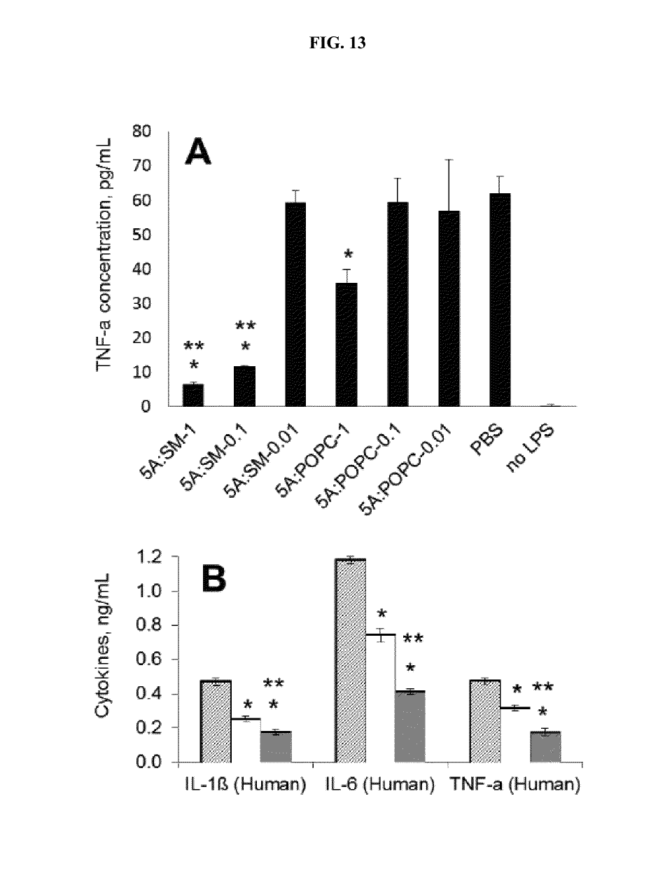

The effects of phospholipid composition on the anti-inflammatory properties of rHDL were examined in two separate experiments. First, mouse peritoneal macrophages were harvested, plated and pre-incubated with 5A-SM or 5A-POPC at 0.01, 0.1, and 1 mg/ml concentrations for 18 h. Following incubation, the media was replaced with fresh media without rHDL and cytokine release was stimulated by addition of LPS. Both lipid formulations of rHDL exhibited dose-dependent inhibition of cytokine release, although 5A-SM appeared to be significantly more potent than 5A-POPC (FIG. 13A). Both the high (1 mg/mL) and mid dose (0.1 mg/mL) of 5A-SM almost completely inhibited TNF-α release, while only the 1 mg/ml dose of 5A-POPC showed some protection. It is known that HDL can physically bind and neutralize LPS; however, this protection mechanism is unlikely to be applicable here, because the rHDL containing media was removed and a new media was added prior to LPS stimulation.

The ability of 5A-SM- and 5A-POPC-HDL to inhibit cytokine release in whole human blood stimulated by addition of PHA was also examined. The 5A-POPC and 5A-SM particles were added at 0, 0.01 and 0.1 mg/mL and pre-incubated for 1 h. Cytokine release was stimulated with PHA and the levels of IL-1β, IL-6 and TNF-α were determined (FIG. 13B). Similar to the mouse macrophages results, both rHDL exhibited dose-dependent inhibition of cytokine release relative to buffer control (p<0.005 for 5A-SM, and p<0.05 for 5A-POPC), and once again 5A-SM offered better protection relative to 5A-POPC (p<0.01).

3.6 Cholesterol Mobilization and Esterification in vivo

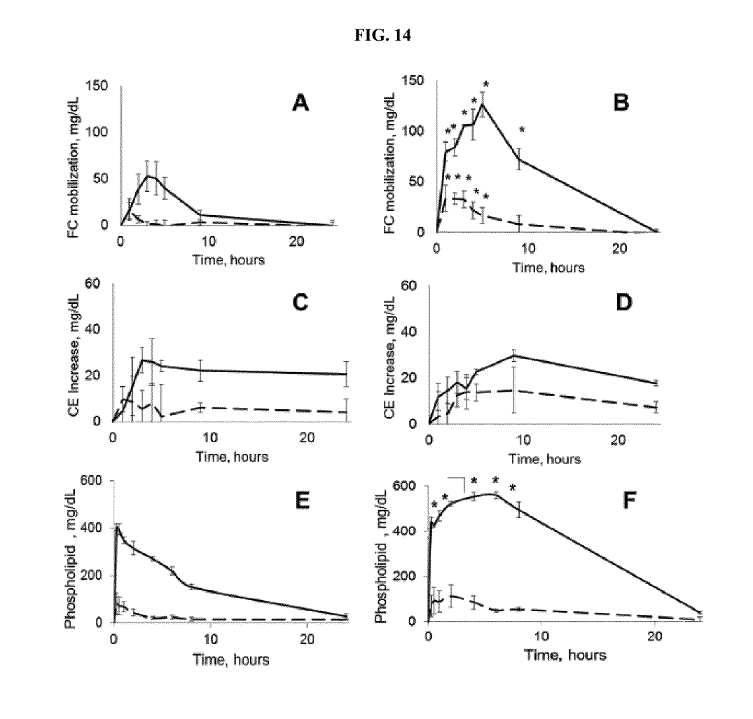

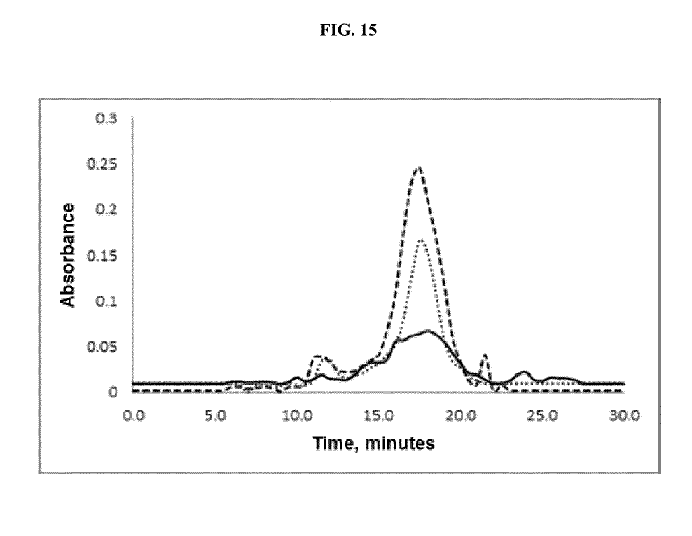

A rat dose-response study was performed in order to evaluate if apparent differences between 5A-SM- and 5A-POPC-HDL in cholesterol efflux in vitro translate to significant differences in cholesterol mobilization in vivo. Sprague Dawley rats received 30 and 100 mg/kg doses of 5A-POPC- or 5A-SM-HDL by intravenous infusion. The dose was based on the peptide content of the dosing solutions. The increase in plasma free cholesterol (FC) (A, B) and cholesterol ester (CE) (C, D) and phospholipid levels (E, F) are plotted in FIG. 14. The amount of mobilized free cholesterol was proportional to the injected dose and reached a maximum within 1-3 h post-infusion, depending on the dose. Most of the mobilized cholesterol appeared to be unesterified. Statistically significant differences in FC increase between 5A-POPC and 5A-SM was observed for 30 and 100 mg/kg dose at almost all time points (p<0.05 noted in FIG. 14). Chromatographic separation of plasma lipoproteins 30 minutes following rHDL dosing revealed the presence of mobilized cholesterol in its entirety in the HDL fraction as shown in FIG. 15. The analysis confirmed higher levels of mobilized cholesterol in HDL fraction for 5A-SM infusion relative to 5A-POPC. Approximately 2-3 times more cholesterol accumulated in the plasma compartment per dose of injected 5A-SM-HDL relative to 5A-POPC-HDL. The maximum FC mobilization was obtained 3 h post-dose for 100 mg/kg and when FC levels increased from baseline by 105.6 mg/dL for 5A-SM-HDL and by 53.3 mg/dL for 5A-POPC-HDL (p<0.01 for two rHDL). The baseline level of plasma FC was 30±5 mg/dL, thus 30 and 100 mg/kg infusion of 5A-SM corresponded to 107% and 387% increase for plasma FC levels. Same doses of 5A-POPC resulted in 53% and 189% increase for plasma FC.

The administration of 30 and 100 mg/kg doses of rHDL (based on 5A peptide amount) corresponds to infusion of 37.5 and 125 mg/kg of either POPC or SM resulting in a significant increase in total circulated plasma phospholipid (PL) levels. The level rat plasma PL prior to infusion is approximately 100-125 mg/mL. The administration of rHDL results in initial PL increase by approximately 70-80 mg/mL and 400-440 mg/mL for 30 and 100 mg/kg doses, respectively. The initial PL increase is similar for both 5A-SM and 5A-POPC. While for 5A-POPC infusion the PL lipid levels decrease according to first-order elimination, whereas for 5A-SM a small additional increase in circulating PL is observed for the first 2-6 hours depending on the dose. The differences in PL levels were statistically significant for 5A-SM and 5A-POPC for 100 mg/kg dose 2-8 hours post administration (p<0.05). Overall, the circulating PL levels remain elevated for a longer duration for 5A-SM administration relative to 5A-POPC most likely due to slower metabolism of SM relative to POPC. This slower PL elimination correlates with longer circulation of mobilized FC (FIGS. 14. A and B). It is important to note that the plasma PL levels return to pre-dose levels 24 hours post administration for both rHDL compositions.

Cholesterol mobilization is the first important step in reverse cholesterol transport pathway; however, it is important to note that the lipid composition of the rHDL particles also affected cholesterol esterification by lecithin-cholesterol acyltransferase (LCAT), the second step in reverse cholesterol transport (11, 31). SM, unlike phosphatidylcholines, such as POPC, is not a substrate for LCAT and some reports indicate that SM inhibits LCAT activity (6). The increase in cholesterol ester (CE) was plotted at each time point following the infusion for each dose of 5A-SM- and 5A-POPC-HDL, as shown in FIGS. 14C and 14D. The dose dependent increase in CE formation was observed for both 5A-SM and 5A-POPC; however, the absolute amount of CE increase was lower than for FC, indicating that only a small fraction of mobilized FC converts to CE for both HDL compositions. These were no statistically significant differences in CE increase between 5A-POPC and 5A-SM. The CE increase was similar for 100 mg/kg dose of 5A-POPC and 5A-SM, as well as 30 mg/kg 5A-SM. These results indicate that rapid mobilization of large amount of FC likely saturates the endogenous LCAT esterification capacity.

3.7 Effect of 5A-POPC and 5A-SM rHDL on Atherosclerosis Regression in Mice

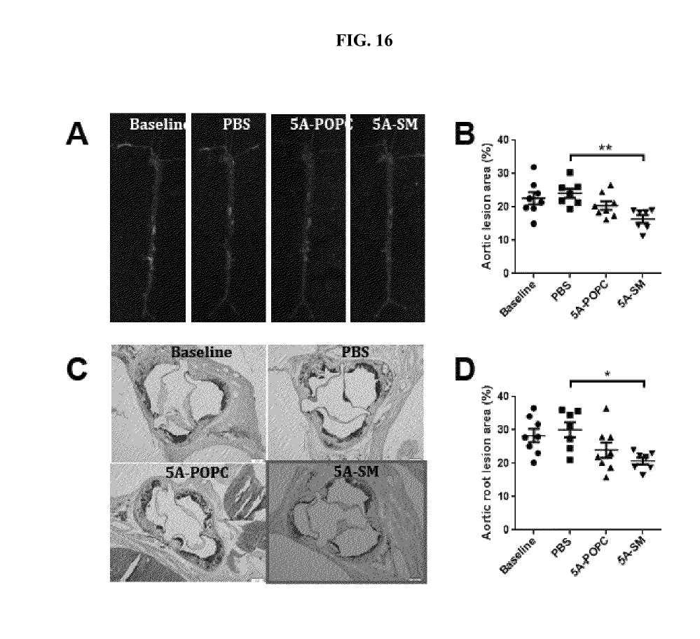

The ability of rHDL to reduce the atherosclerotic burden was evaluated for both 5A-POPC and 5A-SM rHDL in apoE−/− mice. ApoE−/− mice were placed on high-fat diet to develop atherosclerosis (7-8 animals per group). The baseline control group was sacrificed prior to the start of treatment, while the other three groups receive intraperitoneal injections of 50 mg/kg 5A-SM, 5A-POPC, or equivalent volume of PBS administered 3 times weekly for 6 weeks. Following the conclusion of treatment regimens, animals were sacrificed and whole aortas were excised for plaque area analysis by oil red O staining (FIG. 16). The baseline en face atheroma area was 22.5% and it increased slightly over the treatment period for the placebo group to 24.1% (FIG. 16 A-B). In contrast, the atheroma area was reduced to 20.4% and 16.4% following the treatment with 5A-POPC and 5A-SM, respectively. The relative percent reductions of atheroma area by 5A-SM and 5A-POPC were 28% and 10% compared to baseline. There were no statistically significant differences between 5A-SM and 5A-POPC groups likely due to the small number of animals and short duration of treatment. The plaque reduction was statistically significant for 5A-SM-rHDL relative to PBS with a p-value <0.01. Similar results were obtained for aortic root lesions (FIG. 16 C, D). The atheroma area following placebo treatment was 30.0% and it was reduced to 24.0% and 20.7% following 5A-POPC and 5A-SM treatment, respectively. The aortic root lesion reduction was statistically significant for 5A-SM treatment relative to PBS (p<0.05). These results suggest that the phospholipid composition of rHDL does indeed affect both cholesterol mobilization in vivo and the anti-atherosclerotic potency of rHDL in the apoE−/− murine model.

### 4. Discussion

The most important finding of this study is the manner and extent to which alteration of rHDL phospholipid composition can alter the biological properties of rHDL, in regard to its cholesterol efflux ability and anti-inflammatory effect. It was found that 5A-SM effluxes more cholesterol by SR-BI and ABCG1 mechanisms relatively to 5A-POPC, and this ability is translated to greater cholesterol efflux capacity by plasma following infusion of rHDLs. ABCA1 efflux was not as much affected by lipid composition, as it is largely driven by lipid-free ApoA-I or the ApoA-I peptide component of HDL. The cholesterol efflux following addition of peptide-free lipids Addition of 5A-POPC and 5A-SM to human and rat plasma resulted in lipoprotein remodeling with notable formation of pre-β HDL particles. The relative percent of formed pre-β HDL was higher for 5A-SM relative to 5A-POPC. The difference between cholesterol efflux ability by ABCG1 and SR-BI for the two rHDL lipid formulations was translated into notable difference in FC efflux in vivo. The 5A-SM-HDL mobilized roughly twice the amount of cholesterol relative to 5A-POPC-HDL at the same doses (FIG. 14). The mobilized cholesterol was found in the HDL fraction following chromatographic separation of VLDL, LDL and HDL of rat plasma sample 30 minutes post rHDL infusions (FIG. 15). The absolute value of cholesterol ester increase was similar for both rHDL compositions at high doses, indicating that availability of the endogenous LCAT might be a limiting factor for FC conversion to CE. The conversion of FC to CE following administration of SM-based rHDL was surprising, because SM is not a substrate for LCAT indicating that endogenous phosphatidylcholine species are likely to incorporate into SM-based rHDL during remodeling in vivo. Indeed, at the same level of cholesterol mobilization after dosing of either 100 mg/kg of 5A-POPC or 30 mg/kg 5A-SM, roughly same percent of FC was converted to CE. All mobilized cholesterol was successfully eliminated by 24 hours post rHDL infusion even after administration of 5A-SM at 100 mg/mL, as lipoprotein parameters returned to a baseline levels. This indicates that rHDL mobilized cholesterol is most likely eventually eliminated by the liver either as cholesterol ester or as unesterified cholesterol. A control experiment to explore cholesterol efflux in vitro and cholesterol mobilization in vivo following administration of POPC or SM vehicles without addition of ApoA-I mimetic peptide was not performed in the current study. The administration of lipid vehicles results in cholesterol mobilization and endothelial function modulation and the relative efficacy depends on the size and lamellar properties of administered lipid vehicles (32, 33).

The anti-inflammatory properties of rHDL are also affected by lipid composition; 5A-SM was found to inhibit cytokine release in response to LPS more effectively than 5A-POPC. The anti-inflammatory activity differences between 5A-SM and 5A-POPC could be potentially related to the differences in alteration of cellular membrane composition by either passive or SR-BI mediated efflux. The SR-BI mediated efflux from the endothelial cells is known to cause activation of eNOS by kinase signaling (34). It is also possible that some of anti- and pro-inflammatory activity of rHDL is due to small amounts of signaling lipid impurities present in SM and POPC raw materials, such as sphingosine-1-phosphate (SIP), sphingosyl-phosphorylcholine (SPC) or lysophosphatidylcholine (LPC), or these could be generated after cellular uptake by rHDL. These molecules are known to be potent bioactive signaling molecules and could impact on the inflammatory cascade (34). Another potential mechanism of 5A-POPC and 5A-SM related anti-inflammatory activity could be mediated by ABCA1 efflux (35) and related to induction of activating transcription factor 3 (ATF3) transcription modulator (36). The causes for relative differences between 5A-POPC and 5A-SM anti-inflammatory activities pertaining to these mechanisms have not yet been explored.

Similar differences between SM- and PC-based rHDL were reported in the several past studies. Proapolipoprotein A-I (proApoA-I) formulated with POPC showed approximately 10-fold lower free cholesterol mobilization in the HDL fraction relative to proApoA-I-SM-HDL, following the infusion into New Zealand White rabbits at 15 mg/kg doses (24). The comparison of in vitro cholesterol efflux by ApoA-I and ApoA-I-Milano formulated with either 1,2-dimyristoyl-rac-glycero-3-phosphocholine (DMPC) or SM was conducted in CHO cells, J774 macrophages and BHK cells transfected with ABCA1, non-functional ABCA1 mutant (W590S) and ABCG1 (9). SM-based rHDL exhibited higher cholesterol efflux than DMPC-based rHDL in all cell systems tested, and the difference was most significant in ABCG1 expressing cells. The phospholipid composition has less effect on ABCA1 mediated efflux, and ApoA-I-Milano-based rHDL was a slightly better cholesterol acceptor relative to ApoA-I-based rHDL. These findings are consistent with ABCA1 and ABCG1 efflux results in our study, using the 5A mimetic peptide.

The effects of phosphatidylcholine, fatty acid saturation, and addition of SM to rHDL on cholesterol efflux from J774 macrophages and esterified cholesterol uptake by HepG2 cells were also evaluated by Marmillot et al. ((2007) Metabolism, 56(2): 251-9). In general, the highest cholesterol efflux was observed for more saturated lipid based rHDL, such as fully saturated 1,2-distearoyl-PC (DSPC, 18:0) and 1,2-dioleoyl PC (DOPC, 18:1-mono-unsaturated). The incorporation of egg PC (unsaturated natural mixture), 1,2-dilinoleoyl PC (DLPC, 18:2) and 1,2-dilinolenoyl (DLnPC, 18:3) caused a significant decrease in efflux capacity. The addition SM increased cholesterol efflux. The CE uptake by HepG2 cells was also higher for rHDL containing more saturated lipids (50-100% of DSPC), while less uptake was observed for egg york PC (mostly unsaturated), DOPC, DLPC and DLnPC. Addition of SM at 40-80% of total lipid decreased the CE uptake by rHDL containing unsaturated lipids, but not for DSPC-based rHDL

Another study of rHDL lipid composition on cholesterol efflux from mouse L-cell fibroblasts was performed by Davidson et al. but gave slightly different results (7). The SM-based rHDL exhibited slightly lower efflux capacity than unsaturated POPC and DOPC-based rHDL. The rHDL based on saturated PC, such as DSPC (18:0), DPPC (18:0), DMPC (14:0) and 1-palmitoyl-2-stearoyl PC (PSPC; 16:0, 18:0), exhibited lower cholesterol efflux than HDL from POPC and DOPC. This efflux system examines physical uptake of free cholesterol from cell membranes similar to the mock efflux measurement in this study. The higher uptake by unsaturated lipids was attributed to the liquid crystalline state of unsaturated lipids at 37° C. However, the duration of rHDL incubation with cells, cell type, and rHDL concentration in the media were different in all the aforementioned studies, thus limiting direct comparison. It is important to note that commercially available sphingomyelins are all natural mixtures of lipids purified from different sources. Egg yolk SM, which is composed primary of palmitoyl acid (16:0, over 80%) was used in our study, while bovine brain SM (˜50% of 18:0 and ˜20% of 24:1) was used by Marmillot et al. and Davidson et al. (7, 8). Similar to PC, the fatty acid chain length and saturation is expected to affect the cholesterol binding of the SM bilayer and efflux capacity of SM-based rHDL

An important issue is if the greater cholesterol mobilization efficiency by SM-based rHDL relative to POPC-based rHDL will result in anti-atherosclerotic efficacy at lower doses. In this study, a greater anti-atherosclerotic activity of 5A-SM relative to 5A-POPC following administration of eighteen doses of 50 mg/kg of rHDL was observed. Some of our previous data is also supportive of greater activity of SM containing rHDL relative to POPC-based (26). In two independent studies 4-month-old apoE−/− mice on a normal chow diet were injected with 30 mg/kg of: i) 5A:POPC-HDL or POPC liposome control; and ii) 5A-SM-DPPC-HDL or saline control for 13 weeks (3 times a week) and then analyzed for percentage of surface area covered by aortic plaque. The atheroma area was smaller following 5A-SM-DPPC-HDL treatment (˜3.5%) relative to 5A-POPC-HDL treatment (˜6%). Injections of both rHDL showed statistically significant improvement relative to placebo or POPC liposome controls (26). The animal experiment design in the current study involved feeding apoE−/− mice a high-fat diet for 14 weeks followed by an acute 6-week treatment. The mean atheroma areas were 16.4%, 20.4% and 24.1% following 5A-SM-HDL, 5A-POPC-HDL and placebo treatments, respectively. While the atheroma area was lower following 5A-SM treatment relative to 5A-POPC the difference was not statistically significant. This lack of statistically significant difference could be attributed to the low number of animals (7-8/group) and short duration of treatment (6 weeks)

Another critical question is the importance of effective interaction of rHDL with LCAT for successful reduction of atheroma. While SM is not a substrate of LCAT, fully saturated PC, such as DPPC, is capable of serving as a substrate for LCAT and, like SM, is similar in its strong cholesterol binding properties. The unsaturated PC, such as POPC and DLPC, are reported to be more efficient substrates for LCAT but unsaturation results in weaker cholesterol efflux. It may be preferable to investigate the effect of lipid composition cholesterol esterification in vivo using either full-length ApoA-I or ApoA-I mimetic peptides optimized specifically for LCAT activation. The results of this study are significant because they shift the focus of rHDL research from protein or peptide component to an equally important phospholipid component. While several rHDL products have already been translated to early stage clinical trials, the doses required for clinical efficacy are relatively large (40 mg/kg and 45 mg/kg), requiring as much as 5 grams of protein per infusion (4, 5, 15, 16). Administration of such high doses has been associated with toxicity, which may have resulted from various impurities, including: cholate used in preparation sHDL particles, oxidized lipids, potentially immunogenic ApoA-I aggregates, bacterial proteins, DNA and endotoxins from recombinant processing during manufacturing of ApoA-I. A high dose of 80 mg/kg of CSL-111 also had to be discontinued in a Phase 2 study due to toxicity (5). High doses also require the manufacture of large quantities of very pure protein or ApoA-I mimetic peptide and rHDL nanoparticles, which is technically difficult and costly. Hence, the ability to attenuate pharmacological efficacy of rHDL by optimization of its phospholipid composition could potentially lead to the creation of new rHDL therapeutics that are effective at a fraction of the currently used clinical doses

The end goal of rHDL infusion therapy is to rapidly remove excess cholesterol from arterial plaque and reduce the risk of future heart attack and, therefore, the ability of rHDL to mimic all functions of natural HDL i.e., endothelial cell layer penetration, cholesterol efflux from foam cells, anti-inflammatory and anti-oxidant functions, interaction with LCAT for conversion of FC to CE, and transport of mobilized and esterified cholesterol to the liver for elimination is highly important. As this study and previous published literature indicate, all of these functions of rHDL are potentially affected by its phospholipid composition. Therefore, understanding the mechanism of how lipid composition alters efficacy and safety of rHDL is critical for successful clinical translation of this novel class of cardiovascular drugs.

The examples set forth above are provided to give those of ordinary skill in the art a complete disclosure and description of how to make and use the embodiments of the compositions, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

### REFERENCES

Amar, M. J. A., D'Souza, W., Turner, S., Demosky, S., Sviridov, D., Stonik, J., Luchoomun, J., Voogt, J., Hellerstein, M., Sviridov, D., Remaley, A. T. (2010) 5A apolipoprotein mimetic peptide promotes cholesterol efflux and reduces atherosclerosis in mice. J Pharmacol Exp Ther, 334: 634-641.
Asztalos, B. F., Tani, M, Schaefer, E. J. (2011) Metabolic and functional relevance of HDL subspecies. Curr Opin Lipidol, 22(3): 176-185.
Badimon, J. J., Badimon, L., Fuster, V. (1990) Regression of atherosclerotic lesions by high density lipoprotein plasma fraction in the cholesterol-fed rabbit. J Clin Invest, 85(4): 1234-41.
Bhattacharya, S., H. Zhang, et al. (2008). “Solution structure of a hydrocarbon stapled peptide inhibitor in complex with monomeric C-terminal domain of HIV-1 capsid.” J BioI Chem 283(24): 16274-16278.
Bielicki, J. K., H. Zhang, et al. (2010). “A new HDL mimetic peptide that stimulatescellular cholesterol efflux with high efficiency greatly reduces atherosclerosis in mice.” J Lipid Res 51(6): 1496-1503.
Bloedon, L. T., R. Dunbar, et al. (2008). “Safety, pharmacokinetics, and pharmacodynamics of oral apoA-I mimetic peptide D-4F in high-risk cardiovascular patients.” J Lipid Res 49(6): 1344-1352.
Brewer, H. B., Jr., A. T. Remaley, et al. (2004). “Regulation ofplasma highdensity lipoprotein levels by the ABCA1 transporter and the emerging role of high-density lipoprotein in the treatment ofcardiovascular disease.” Arterioscler Thromb Vasc Biol 24(10): 1755-1760.
Buga, G. M., J. S. Frank, et al. (2006). “D-4F decreases brain arteriole inflammation and improves cognitive performance in LDL receptor-null mice on a Western diet.” J Lipid Res 47(10): 2148-2160.
Camont, L., Lhomme, M., Rached, F., Le Goff, W., Nègre-Salvayre, A., Salvayre, R., Calzada, C., Lagarde, M., Chapman, M. J., Kontush, A. (2013) Small, dense high-density lipoprotein-3 particles are enriched in negatively charged phospholipids: relevance to cellular cholesterol efflux, antioxidative, antithrombotic, anti-inflammatory, and antiapoptotic functionalities. Arterioscler Thromb Vasc Biol, 33(12): 2715-2723.
Dasseux, J. L. H, Rea, T. J., Shenderova, A. A. Method of treating dyslipidemic disorders. U.S. Pat. No. 9,941,20 B2.
De Nardo, D., Labzin, L. I., Kono, H, Seki, R., Schmidt, S. V., Beyer, M., Xu, D., Zimmer, S., Lahrmann, C., Schildberg, F. A., Vogelhuber, J., Kraut, M., Ulas, T., Kerksiek, A., Krebs, W., Bode, N., Grebe, A., Fitzgerald, M. L., Hernandez, N. J., Williams, B. R. G., Knolle, P., Kneilling, M., Röcken, M., Liitjohann, D., Wright, S. D., Schultze, J. L., Latz, E. (2014) High-density lipoprotein mediates anti-inflammatory reprogramming of macrophages via the transcriptional regulator ATF3. Nat Immunol, 15(2): 152-160.
Davidson, W. S., Gillotte, K. L., Lund-Katz, S., Johnson, W. J., Rothblat, G. H., Phillips, M. C. (1995) The effect of high density lipoprotein phospholipid acyl chain composition on the efflux of cellular free cholesterol. J. Biol. Chem., 270: 5882-5890.
Di Bartoloa, B. A., Nicholls, S. J., Bao, S., Rye, K. A., Heather, A. K., Barter, P. J., Bursill, C. (2011) The apolipoprotein A-I mimetic peptide ETC-642 exhibits anti-inflammatory properties that are comparable to high density lipoproteins, Atherosclerosis, 217: 395-400.
D'Souza, W., J. A. Stonik, et al. (2010). “Structure/function relationships of apolipoprotein a-I mimetic peptides: implications for antiatherogenic activities of high-density lipoprotein.” Circ Res 107(2): 217-227.
Eriksson, M., Carlson, L. A., Miettinen, T. A., Angelin, B. (1999) Stimulation of fecal steroid excretion after infusion of recombinant proapolipoprotein A-I. Potential reverse cholesterol transport in humans. Circulation, 100(6): 594-598.
Frank, P. G. and Y. L. Marcel (2000). “Apolipoprotein A-I: structure-function relationships.” J Lipid Res 41(6): 853-872.
Freeman, L. A. (2013) Native-native 2D gel electrophoresis for HDL subpopulation analysis. Methods Mol Biol., 1027:353-67.
Freeman, L. A. (2013) Western blots. Methods Mol Biol., 1027:369-85.
Henchey, L. K., A. L. Jochim, et al. (2008). “Contemporary strategies for the stabilization of peptides in the alpha-helical conformation.” Curr Opin Chem Biol 12(6): 692-697.
Hippalgaonkar, K., Majumdar, S., Kansara, V. (2010) Injectable lipid emulsions—advancements, opportunities and challenges. AAPS PharmSciTech, 11(4): 1526-1540.
Khera, A. V., M. Cuchel, et al. (2011). “Cholesterol efflux capacity, high-density lipoprotein function, and atherosclrosis.” N Engl J Med 364(2): 127-135.
Kim, Y. W., P. W. Kutchukian, G. L. Verdine (2010). “Introduction of a hydrocarbon, i,i+3 staples into alpha-helices via ring-closing olefin metathesis. Org Lett 12(13): 3046-9.
Klon, A. E., J. P. Segrest, et al. (2002). “Comparative models for human apolipoprotein A-I bound to lipid in discoidal high-density lipoprotein particles.” Biochemistry 41(36): 10895-10905.
Kontush, A., Therond, P., Zerrad, A., Couturier, M., Négre-Salvayre, A., de Souza, J. A., Chantepie, S., Chapman, M. J. (2007) Preferential sphingosine-1-phosphate enrichment and sphingomyelin depletion are key features of small dense HDL3 particles: relevance to antiapoptotic and antioxidative activities. Arterioscler Thromb Vasc Biol., 27: 1843-1849.
Kontush, A., Lhomme, M., Chapman, M. J. (2013) Unravelling the complexities of the HDL lipidome. J Lipid Res, 54: 2950-2962.
Kutchukian, P. S., J. S. Yang, et al. (2009). “All-Atom Model for Stabilization of alpha-Helical Structure in Peptides by Hydrocarbon Staples.” Journal of the American Chemical Society 131(13): 4622-4627.
Lund-Katz, S. and M. C. Phillips (2010) “High density lipoprotein structurefunction and role in reverse cholesterol transport.” Sub cell Biochem 51: 183-227.
Ma, C. I., Beckstead, J. A., Thompson, A., Hafiane, A., Wang, R. H., Ryan, R. O., Kiss, R. S. (2012) Tweaking the cholesterol efflux capacity of reconstituted HDL. Biochem Cell Biol, 90(5): 636-45.
Marchesi, M., Booth, E. A., Davis, T, Bisgaier, C. L., Lucchesi, B. R. (2004) Apolipoprotein A-I-Milano and 1-palmitoyl-2-oleoyl phosphatidylcholine complex (ETC-216) protects the in vivo rabbit heart from regional ischemia-reperfusion injury. J Pharmacol Exp Ther, 311(3): 1023-31.
Marmillot, P., Patel, S., Lakshman, M. R. (2007) Reverse cholesterol transport is regulated by varying fatty acyl chain saturation and sphingomyelin content in reconstituted high-density lipoproteins. Metabolism, 56(2): 251-9.
Mendez, A. J., G. M. Anantharamaiah, et al. (1994). “Synthetic amphipathic helical peptides that mimic apolipoprotein A-I in clearing cellular cholesterol.” J Clin Invest 94(4): 1698-170.5.
Mineo, C., Deguchi, H., Griffin, J. H, Shaul, P. W. (2006) Endothelial and antithrombotic actions of HDL. Circ Res, 98:1352-1364.
Nanjee, M. N., Doran, J. E., Lerch, P. G., Miller, N. E. (1999) Acute effects of intravenous infusion of ApoA1/phosphatidylcholine discs on plasma lipoproteins in humans. Arterioscler Thromb Vasc Biol, 19(4): 979-89.
Navab, M., S. T. Reddy, et al. (2011). “HDL and cardiovascular disease: atherogenic and atheroprotective mechanisms.” Nat Rev Cardiol. 8(4): 222-232.
Navab, M., P. Ruchala, et al. (2009) “A novel method for oral delivery of apolipoprotein mimetic peptides synthesized from all L-amino acids.” Lipid Res 50(8): 1538-1547.
Nestor, Y. J. (2009) “The medicinal chemistry of pepties.” Curr Med Chem 16(33):4399-418.
Newton, R. S., Krause, B. R. (2002) HDL therapy for the acute treatment of atherosclerosis. Atheroscler Suppl, 3(4): 31-8.
Nissen, S. E., Tsunoda, T., Tuzcu, E. M., Schownhagen, P., Yasin, M., Eaton, G. M., Laurer, M. A., Sheldon, W. S., Grines, C. L, Halpern., S., Crowe, T., Blankenship, J. C., Kerensky, R. (2003) Effect of recombinant ApoA-IMilano on coronary atherosclerosis in patients with acute coronary syndromes: a randomized controlled trial. JAMA, 290: 2292-2300.
Nissen, S. E. (2005) “Effect of intensive lipid lowering on progression of coronary atherosclerosis: evidence for an early benefit from the Reversal of Atherosclerosis with Aggressive Lipid Lowering (REVERSAL) trial.” Am J. Cardio 96(5A): 61F-68F.
Ohvo-Rekilä, H., Ramstedt, B., Leppimiki, P., Slotte, J. P. (2002) Cholesterol interactions with phospholipids in membranes. Prog Lipid Res, 41(1): 66-97.
Osei-Hwedieh, D. O., M. Amar, et al. (2011) “Apolipoprotein mimetic peptides: Mechanisms of action as anti-atherogenic agents.” Pharmacol Ther 130(1):83-91.
Panagotopulos, S. E., S. R. Witting, et al. (2002) “The role of apolipoprotein A-I helix 10. in apolipoprotein-mediated cholesterol efflux via the ATP binding cassette transporter ABCA1.” J Biol Chem 277(42): 3947739484.
Parks, J. S., Huggins, K. W., Gebre, A. K., Burleson, E. R. (2000) Phosphatidylcholine fluidity and structure affect lecithin:cholesterol acyltransferase activity. J Lipid Res, 41: 546-553.
Phillips, M. C., K. L. Gillotte, et al. (1998). “Mechanisms of high density lipoprotein-mediated efflux of cholesterol from cell plasma membranes.” Atherosclerosis 137 Suppl: S13-17.
Ramstedt, B., Slotte, J. P. (1999) Interaction of cholesterol with sphingomyelins and acyl-chain-matched phosphatidylcholines: A comparative study of the effect of the chain length. Biophys. J. 76: 908-915.
Remaley, A. T., F. Thomas, et al. (2003). “Synthetic amphipathic helical peptides promote lipid efflux from cells by an ABCA1˜dependent and an ABCA1˜independent pathway.” J Lipid Res 44(4): 828-836. Remaley, A. T., M. Amar, et al. (2008) “HDL-replacement therapy: mechanism of action, types of agents and potential clinical indications.” Expert Rev Cardiovasc Ther 6(9): 120.3-1215.
Remaley A T, W. G. (2008). “High-density lipoprotein: what is the best way to measure its antiathrerogenic potential.” Expert Opinion on Medical Diagnostics 2(7): 773-788.
Remaley, A. T., Amar, A., Sviridov, D. (2009) HDL-replacement therapy: mechanism of action, types of agents and potential clinical indications. Expert Rev Cardiovasc Ther, 6(9): 1203-15.
Rodrigueza, W. V., Mazany, K. D., Essenburg, A. D., Pape, M. E., Rea, T. J., Bisgaier, C. L., Williams, K. J. (1997) Large versus small unilamellar vesicles mediate reverse cholesterol transport in vivo into two distinct hepatic metabolic pools. Implications for the treatment of atherosclerosis. Arterioscler Thromb Vasc Biol., 17(10): 2132-9.
Rothblat, G. H. and M. C. Phillips (2010). “High-density lipoprotein heterogeneity and function in reverse cholesterol transport.” Curr Opin Lipid 21(3): 229-238.
Rye, K. A., Hime, N. J., Barter, P. J. (1996) The Influence of sphingomyelin on the structure and function of reconstituted high density lipoproteins. J. Biol Chem, 271: 4243-4250.
Sethi, A. A., M. Amar, et al. (2007). “Apolipoprotein AI mimetic peptides: possible new agents for the treatment ofatherosclerosis.” Curr Opin Investig Drugs 8(3): 201-212.
Sethi, A. A., Stonik, J. A., Thomas, F., Demosky, S. J., Amar, M., Neufeld, E., Brewer, H. B., Davidson, W. S., D'Souza, W., Sviridov, D., Remaley, A. T. (2008) Asymmetry in the lipid affinity of bihelical amphipathic peptides. A structural determinant for the specificity of ABCA1-dependent cholesterol efflux by peptides. J Biol Chem, 283:32273-32282.
Shaw, J. A., A. Bobik, et al. (2008). “Infusion of reconstituted high-density lipoprotein leads to acute changes in human atherosclerotic plaque.” Circ Res 103(10): 1084-1091.
Silva. L. C., Futerman, A. H., Prieto, M. (2009) Lipid raft composition modulates sphingomyelinase activity and ceramide-induced membrane physical alterations. Biophys. J. 96(8): 3210-22
Smith, L. c., H. J. Pownall, et al. (1978). “The plasma lipoproteins: structure and metabolism.” Annu Rev Biochem 47: 751˜757.
Sparks, D. L., Davidson, W. S., Lund-Katz, S., Phillips, M. C. (1995) Effects of the neutral lipid content of high density lipoprotein on apolipoprotein AI structure and particle stability. J. Biol. Chem., 270: 26910-26917.
Tabet, F., Remaley, A. T., Segaliny, A. I., Millet, J., Yan, L., Nakhla, S., Barter, P. J., Rye, K. A., Lambert, G. (2010) The 5A apolipoprotein A-I mimetic peptide displays anti-inflammatory and antioxidant properties in vivo and in vitro. Arterioscler Thromb Vasc Biol, 30: 246-252.
Tardif, J. C., Gregoire, J., L'Allier, P. L., Ibrahim, R., Lespérance, J., Heinonen, T. H., Kouz, S., Berry, C., Basser, R., Lavoie, M.-A., Guertin, M.-C., Rodés-Cabau, J. (2007) Effects of reconstituted high-density lipoprotein infusions on coronary atherosclerosis: a randomized controlled trial. JAMA; 297: 1675-1682.
Tardy, C., Goffinet, M., Boubekeur, N., Ackermann R., Sy, G., Bluteau, A., Cholez, G., Keyserling, C., Lalwani, N., Paolini, J. F., Dasseux, J. L., Barbaras, R., Baron, R. (2014) CER-001, a HDL-mimetic, stimulates the reverse lipid transport and atherosclerosis regression in high cholesterol diet-fed LDL-receptor deficient mice. Atherosclerosis, 232(1): 110-118.
Watson, C. E., N. Weissbach, et al. (2011). “Treatment of patients with cardiovascular disease with L-4F, an apo-A1 mimetic, did not improve select biomarkers of HDL function.” J Lipid Res 52(2): 361-373.
Wool, G. D., C. A. Reardon, et al. (2008). “Apolipoprotein A-I mimetic peptide helix number and helix linker influence potentially anti-atherogenic properties.” J Lipid Res 49(6): 1268-1283.
Williams, K. J., Scalia, R., Mazany, K. D., Rodrigueza, W. V., Lefer, A. M. (2000) Rapid restoration of normal endothelial functions in genetically hyperlipidemic mice by a synthetic mediator of reverse lipid transport. Arterioscler Thromb Vasc Biol., 20:1033-1039.
Yancey, P. G., Bortnick, A. E., Kellner-Weibel. G., de la Llere-Moya, M. Phillips, M., Rothblat, G. H. (2003) Importance of different pathways of cellular cholesterol efflux. Arterioscler Thromb Vasc Biol, 23: 712-719.
Zhu, X., Lee, J. Y., Timmins, J. M., Brown, J. M., Boudyguina, E., Mulya, A., Gebre, A. K., Willingham, M. C., Hiltbold, E. M., Mishra, N., Maeda, N., Parks, J. S. (2008) Increased cellular free cholesterol in macrophage-specific Abcal knock-out mice enhances pro-inflammatory response of macrophages. J. Biol. Chem, 283: 22930-22941.

1. A pharmaceutical formulation comprising the apolipoprotein mimetic peptide 5A of SEQ ID NO:1 and at least one phospholipid.

2. The pharmaceutical formulation of claim 1, wherein said at least one phospholipid is a phospholipid selected from the group consisting of sphingomyelin (SM), 1-palmitoyl-2-oleoyl-sn-glycero-3-phosphocholine (POPC), and a combination thereof.

3. The pharmaceutical formulation of claim 1, wherein said at least one phospholipid is sphingomyelin (SM).

4. The pharmaceutical formulation of claim 1, wherein said at least one phospholipid is 1-palmitoyl-2-oleoyl-sn-glycero-3-phosphocholine (POPC).

5. The pharmaceutical formulation of claim 1, wherein said molar ratio of peptide to phospholipid is 1:3, 1:4, 1:5, 1:6, 1:7, 1:8, 1:9, or 1:10.

6. The pharmaceutical formulation of claim 1, wherein said molar ratio of peptide to phospholipid is 1:7.

7. The pharmaceutical formulation of claim 1, wherein said peptide is complexed with said at least one phospholipid.

8. The pharmaceutical formulation of claim 1, wherein said formulation is lyophilized.

9. The pharmaceutical formulation of claim 1, wherein said formulation has a pH of 5 to 7.

10. The pharmaceutical formulation of claim 1, wherein said formulation has a pH of is about 5, 5.5, 6, 6.5, or 7.

11. A method for generating a pharmaceutical formulation according to claim 1, wherein said method comprises mixing of said peptide with said at least one phospholipid, wherein said mixing occurs at a pH of 5 to 7.

12. The method of claim 11, wherein said pH is about 5, 5.5, 6, 6.5, or 7.

13. The method of claim 11, wherein said peptide and said at least one phospholipid are mixed at a molar ratio of peptide to phospholipid of 1:3, 1:4, 1:5, 1:6, 1:7, 1:8, 1:9, or 1:10.

14. The method of claim 11, wherein said molar ratio of peptide to phospholipid during mixing is 1:7.

15. The method of claim 11, wherein said method further comprises a step of lyophilization.

16. The method of claim 11, wherein said method generates a high-density lipoprotein-like complex.

17. A method of treating a subject in need thereof comprising administering a pharmaceutical formulation according to claim 1.

18. A method of increasing cholesterol efflux in a subject in need thereof comprising administering a pharmaceutical formulation according to claim 1.

19. A method of treating a subject in need thereof comprising administering a pharmaceutical formulation according to claim 1, wherein said pharmaceutical formulation exhibits anti-atherosclerotic activity.

20. A method of increasing pre-β HDL in a subject in need thereof comprising administering a pharmaceutical formulation according to claim 1.

21. A method of treating a subject in need thereof comprising administering a pharmaceutical formulation according to claim 1, wherein said pharmaceutical formulation exhibits anti-inflammatory activity.

22. A method of treating a subject in need thereof comprising administering a pharmaceutical formulation according to claim 1, wherein said pharmaceutical formulation inhibits cytokine release.

23. The method of claim 22, wherein said cytokines are selected from the group consisting of TNF-α, IL-1β, IL-6, and a combination thereof.

24. A method of increasing cholesterol mobilization and/or esterification in a subject in need thereof comprising administering a pharmaceutical formulation according to claim 1.

25. A method of inducing atherosclerosis regression in a subject in need thereof comprising administering a pharmaceutical formulation according to claim 1.

26. A method according to claim 11, comprising administering an additional agent before, after or concurrently with the pharmaceutical formulation.
